SEQUENCE LISTING (42 sequences)

SEQ ID NO: 1 - DNA, 20 bp, Artificial Sequence
  SYNTHETIC PRIMER
ctttcgaagcctttgctctg

SEQ ID NO: 2 - DNA, 20 bp, Artificial Sequence
  SYNTHETIC PRIMER
caggagagcaatttggagga

SEQ ID NO: 3 - DNA, 20 bp, Artificial Sequence
  SYNTHETIC PRIMER
ggtggtggtccccagtaatg

SEQ ID NO: 4 - DNA, 21 bp, Artificial Sequence
  SYNTHETIC PRIMER
accacgtccacaaccttgtct

SEQ ID NO: 5 - DNA, 22 bp, Artificial Sequence
  SYNTHETIC PRIMER
gtggacaaatgcgacgaacccc

SEQ ID NO: 6 - DNA, 20 bp, Artificial Sequence
  SYNTHETIC PRIMER
tcgaaggtcagccagaacag

SEQ ID NO: 7 - DNA, 20 bp, Artificial Sequence
  SYNTHETIC PRIMER
tctcagaggagcctggctaa

SEQ ID NO: 8 - DNA, 20 bp, Artificial Sequence
  SYNTHETIC PRIMER
tgacatctcaattgctccag

SEQ ID NO: 9 - DNA, 22 bp, Artificial Sequence
  SYNTHETIC PRIMER
cctgctgctttgcctacattgc

SEQ ID NO: 10 - DNA, 22 bp, Artificial Sequence
  SYNTHETIC PRIMER
acacacttggcggttctttcgg

SEQ ID NO: 11 - DNA, 20 bp, Artificial Sequence
  SYNTHETIC PRIMER
agcttggcagaagctctgta

SEQ ID NO: 12 - DNA, 20 bp, Artificial Sequence
  SYNTHETIC PRIMER
ccaactgcatccctaagcac

SEQ ID NO: 13 - DNA, 17 bp, Artificial Sequence
  SYNTHETIC PRIMER
gttgtcgacgacgagcg

SEQ ID NO: 14 - DNA, 17 bp, Artificial Sequence
  SYNTHETIC PRIMER
gcacagagcctcgcctt

SEQ ID NO: 15 - DNA, 22 bp, Artificial Sequence
  SYNTHETIC PRIMER
ccaggagcttgaataaaatgaa

SEQ ID NO: 16 - DNA, 22 bp, Artificial Sequence
  SYNTHETIC PRIMER
tgcagtgagaatgatcttcctt

SEQ ID NO: 17 - DNA, 23 bp, Artificial Sequence
  SYNTHETIC PRIMER
atcccagacacacaagcagagag

SEQ ID NO: 18 - DNA, 21 bp, Artificial Sequence
  SYNTHETIC PRIMER
ggctgtgggtttgagtcttct

SEQ ID NO: 19 - DNA, 20 bp, Artificial Sequence
  SYNTHETIC PRIMER
ccctatggagatgacggaga

SEQ ID NO: 20 - DNA, 20 bp, Artificial Sequence
  SYNTHETIC PRIMER
cccagtgctggagaaattgt

SEQ ID NO: 21 - DNA, 21 bp, Artificial Sequence
  SYNTHETIC PRIMER
gcagaagtctggagagccctc

SEQ ID NO: 22 - DNA, 21 bp, Artificial Sequence
  SYNTHETIC PRIMER
tgagatgcagtgttctggtcc

SEQ ID NO: 23 - DNA, 20 bp, Artificial Sequence
  SYNTHETIC PRIMER
tccatgacggtgtcagaact

SEQ ID NO: 24 - DNA, 20 bp, Artificial Sequence
  SYNTHETIC PRIMER
gacccagactggaaagggta

SEQ ID NO: 25 - DNA, 19 bp, Artificial Sequence
  SYNTHETIC PRIMER
cctgcccacgtgttgagat

SEQ ID NO: 26 - DNA, 23 bp, Artificial Sequence
  SYNTHETIC PRIMER
tgatggtcttagattccggattc

SEQ ID NO: 27 - DNA, 20 bp, Artificial Sequence
  SYNTHETIC PRIMER
gtggtagtccccagcaatgt

SEQ ID NO: 28 - DNA, 20 bp, Artificial Sequence
  SYNTHETIC PRIMER
tgctgacctctgcacttgac

SEQ ID NO: 29 - DNA, 20 bp, Artificial Sequence
  SYNTHETIC PRIMER
caaggcaggtttctgaggag

SEQ ID NO: 30 - DNA, 20 bp, Artificial Sequence
  SYNTHETIC PRIMER
gacctggtcaccatcagcat

SEQ ID NO: 31 - DNA, 18 bp, Artificial Sequence
  SYNTHETIC PRIMER
gttgtctcctgcgacttc

SEQ ID NO: 32 - DNA, 19 bp, Artificial Sequence
  SYNTHETIC PRIMER
ggtggtccagggtttctta

SEQ ID NO: 33 - DNA, 20 bp, Artificial Sequence
  SYNTHETIC PRIMER
acacgtccaactcagtttgc

SEQ ID NO: 34 - DNA, 20 bp, Artificial Sequence
  SYNTHETIC PRIMER
cgagcatccgaacgtttgat

SEQ ID NO: 35 - DNA, 24 bp, Artificial Sequence
  SYNTHETIC PRIMER
acttctttttcttgctttcgtggt

SEQ ID NO: 36 - DNA, 20 bp, Artificial Sequence
  SYNTHETIC PRIMER
gcagcagtacgcacacaatc

SEQ ID NO: 37 - DNA, 22 bp, Artificial Sequence
  SYNTHETIC PRIMER
ggggaacttctcctgctagaat

SEQ ID NO: 38 - DNA, 22 bp, Artificial Sequence
  SYNTHETIC PRIMER
ggggaacttctcctgctagaat

SEQ ID NO: 39 - DNA, 23 bp, Artificial Sequence
  SYNTHETIC PRIMER
ctggtgcatatgttcccgggagg

SEQ ID NO: 40 - DNA, 23 bp, Artificial Sequence
  SYNTHETIC PRIMER
gccgtaggccgtgcttccaaggg

SEQ ID NO: 41 - DNA, 28 bp, Artificial Sequence
  SYNTHETIC PRIMER
gcaccgctaaccgaaaccgaaactgtgc

SEQ ID NO: 42 - DNA, 28 bp, Artificial Sequence
  SYNTHETIC PRIMER
gcacagtttcggtttcggttagcggtgc

(19) United States
(12) Patent Application Publication
(10) Pub. No.: US 2024/0109859 A1
(43) Pub. Date: Apr. 4, 2024

(54) ENZYME INHIBITORS AND VIRAL INFECTION THERAPY

(71) Applicant: UNIVERSITY OF SOUTHERN CALIFORNIA, Los Angeles, CA (US)

(72) Inventors: Pinghui FENG, Los Angeles, CA (US); Chao ZHANG, Los Angeles, CA (US); Jun ZHAO, Los Angeles, CA (US); Biancha ESPINOSA, Los Angeles, CA (US); Chao QIN, Los Angeles, CA (US); Ali SAVAS, Los Angeles, CA (US); Youliang RAO, Los Angeles, CA (US); Ting-Yu WANG, Los Angeles, CA (US)

(73) Assignee: UNIVERSITY OF SOUTHERN CALIFORNIA, Los Angeles, CA (US)

(21) Appl. No.: 18/263,582

(22) PCT Filed: Feb. 1, 2022
(86) PCT No.: PCT/US22/14680

§ 371 Date: Jul. 31, 2023

Related U.S. Application Data
(60) Provisional application No. 63/144,214, filed on Feb. 1, 2021.

(51) Int. Cl. C07D 333/38; A61K 9/00; A61K 9/06; A61K 9/20; A61K 9/48; A61K 47/10; A61K 47/12; A61K 47/14; A61K 47/18; A61K 47/26; A61K 47/32; A61K 47/38; A61K 47/44; A61P 31/14; C07C 233/65; C07C 233/75; C07C 233/76; C07D 233/88; C07D 239/94

(52) U.S. Cl. CPC C07D 333/38; A61K 9/0014; A61K 9/0019; A61K 9/06; A61K 9/2013; A61K 9/2018; A61K 9/2027; A61K 9/2054; A61K 9/2059; A61K 9/485; A61K 9/4858; A61K 9/4866; A61K 47/10; A61K 47/12; A61K 47/14; A61K 47/18; A61K 47/26; A61K 47/32; A61K 47/38; A61K 47/44; A61P 31/14; C07C 233/65; C07C 233/75; C07C 233/76; C07D 233/88; C07D 239/94

(57) ABSTRACT

The invention relates to antiviral compounds, compositions comprising antiviral compounds, and methods of use. In particular, the antiviral compounds are cytidine triphosphate synthetase 1 (CTPS1) inhibitors that are effective in treating and/or preventing viral infection and in particular, SARS-CoV-2 infection and/or COVID-19 disease.

### RELATED APPLICATIONS

This application claims priority under 35 U.S.C. § 119(e) to U.S. Provisional Patent Application No. 63/144,214 filed Feb. 1, 2021, which is incorporated herein by reference in its entirety.

### STATEMENT REGARDING FEDERAL FUNDING

This invention was made with government support under Grant No. DE027556, awarded by the National Institutes of Health (NIH). The government has certain rights in the invention.

### BACKGROUND OF THE INVENTION

First reported in December 2019, the coronavirus disease (known as COVID-19) rapidly spread worldwide and became a global pandemic, causing more than 100 million infections and claiming more than 2.16 million lives. The etiological agent of COVID-19 was soon identified as a new coronavirus, severe acute respiratory syndrome coronavirus 2 (SARS-CoV-2). Despite the rapid development of effective vaccine against SARS-CoV-2, COVID-19 patients urgently require post-infection treatment or therapeutics. Current management of severe COVID-19 patients primarily consists of supportive care that optimizes oxygen administration via intubation and mechanical ventilation. Treatment options are limited to repurposed drugs, such as chloroquine and remdesivir, and their clinical effect on severe or critical COVID-19 patients is yet to be established with well controlled trials.

Compared with previous zoonotic coronaviruses including SARS-CoV and MERS-CoV, SARS-CoV-2 is highly infectious and transmissible. Current effort has extensively focused on the entry step that is primarily mediated by the interaction between SARS-CoV-2 spike (S) protein and the human angiotensin-converting enzyme 2 (hACE2). Structural and functional analyses of this receptor-ligand interaction indicate that SARS-CoV-2 S protein evolved better affinity for binding to hACE2 on target cells, partly explaining the highly infectious nature of SARS-CoV-2 during COVID-19 pandemic. However, little is known concerning the viral mechanisms downstream of viral entry that contribute to the infection and pathogenesis of SARS-CoV-2. Innate immune response constitutes the first line of defense against intracellular pathogens such as viruses. To efficiently replicate within an immune-competent host, a virus necessitates to overcome the barrier of host innate immune defense, chiefly mediated by the interferon system. Indeed, previous studies of SARS-CoV-2 infection involving patient samples, model animals and cell lines suggest that SARS-CoV-2 either weakly induces IFNs or inhibits IFN induction. How exactly SARS-CoV-2 interacts with the cellular IFN system remains largely unknown, except sequence analysis comparing SARS-CoV-2 to SARS-CoV and MERS-CoV was used to predict plausible functions of viral proteins.

In addition to overcome innate immune defense, viruses rely on cellular machinery to synthesize macromolecules and biomaterial that assemble into progeny virions. Thus, viruses often activate and redirect cellular biosynthetic activities to facilitate the production of viral components, such as proteins, nucleic acids and lipids that constitute essential building blocks of virions. Central to viral replication is the reprogramming of cellular metabolic processes that are often activated to provide precursors for viral biosynthesis in infected cells. The highly infectious nature of SARS-CoV-2 likely involves molecular interactions that boost the rate-limiting steps of key metabolic pathways to fuel viral replication and subsequent dissemination. Cellular glutamine amidotransferases catalyze the synthesis of nucleotides, amino acids, glycoproteins and an enzyme cofactor (NAD). These enzymes are capable of deamidating key signaling molecules, such as those of innate immune defense, to modulate fundamental biological processes, making them an attractive target for antiviral therapies.

Accordingly, there is a need for medicinal compounds that prevent or reduce deamidation of key signaling molecules to treat viral infections and, in particular, SARS-CoV-2 infections. The present disclosure satisfies these needs.

### SUMMARY OF THE INVENTION

This disclosure provides for certain small molecule enzymatic inhibitor compounds and compositions thereof that are effective in treating viral infections, and in particular, coronavirus infections, and more particularly, infections caused by the SARS-CoV-2 virus.

In some embodiments, the compounds inhibit the enzyme cytidine triphosphate synthetase 1 (CTPS1). In some embodiments, the compounds or a pharmaceutically acceptable salt thereof have the formula I, II, or III:

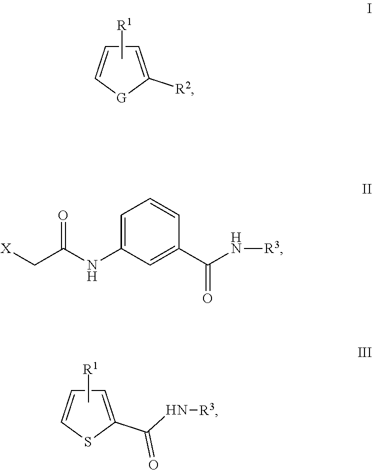

wherein,
G is

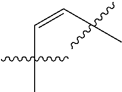 [SMILES: */C=C\*]

### or S;

R¹ is —NH(C═O)CH₂X wherein X is a leaving group;
R² is —(C═O)NR^{a}R³ or —NR^{a}(C═O)R³, wherein R^{a} is H or —(C₁-C₆)alkyl;
R³ is —(C₁-C₆)alkyl, —(C₃-C₆)cycloalkyl, phenyl-R⁴, or 5- or 6-membered heteroaryl; and
R⁴ is —O(C₁-C₆)alkyl, —(C₁-C₆)alkyl, halo, or H;
wherein R¹ is in the beta-position relative to R², and each (C₁-C₆)alkyl moiety is independently saturated or unsaturated and optionally interrupted by a heteroatom.

In some embodiments, the compound or pharmaceutically acceptable salt thereof is one or more of:

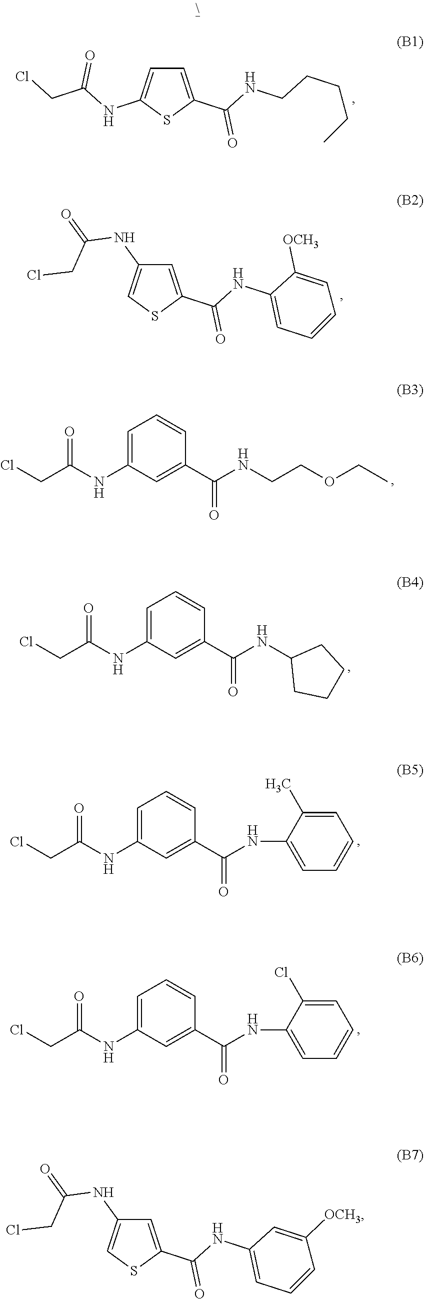

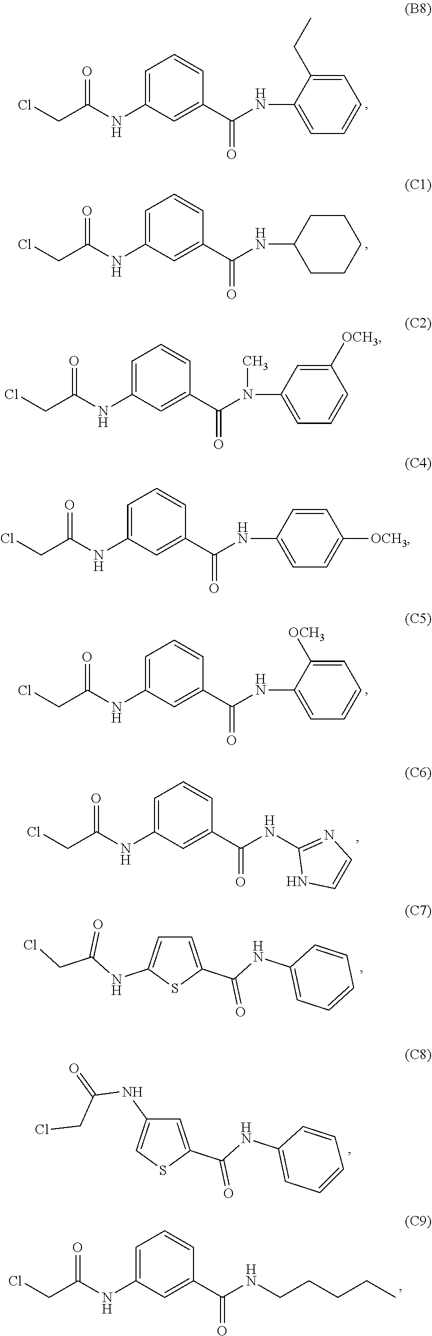

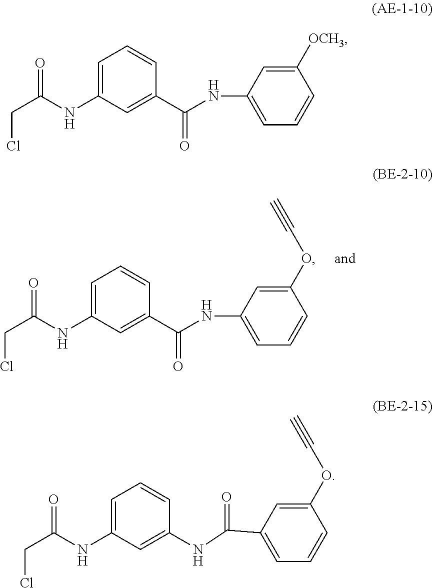 [SMILES: COc1cccc(NC(=O)c2cccc(NC(=O)CCl)c2)c1 ; C#COc1cccc(NC(=O)c2cccc(NC(=O)CCl)c2)c1 ; C#COc1cccc(C(=O)Nc2cccc(NC(=O)CCl)c2)c1]

Some embodiments include a pharmaceutical composition comprising a compound of any one of formula I, II, or III, and a pharmaceutically acceptable excipient.

Embodiments of the disclosure also provide for a method for antiviral treatment comprising administering to a subject in need thereof a therapeutically effective amount of a compound of any one of claims formula I, II, or III, or a pharmaceutically acceptable salt thereof, thereby inhibiting replication of a virus that has infected the subject.

In some embodiments, a composition for use in antiviral treatment comprises one or more compounds or pharmaceutically acceptable salts thereof:

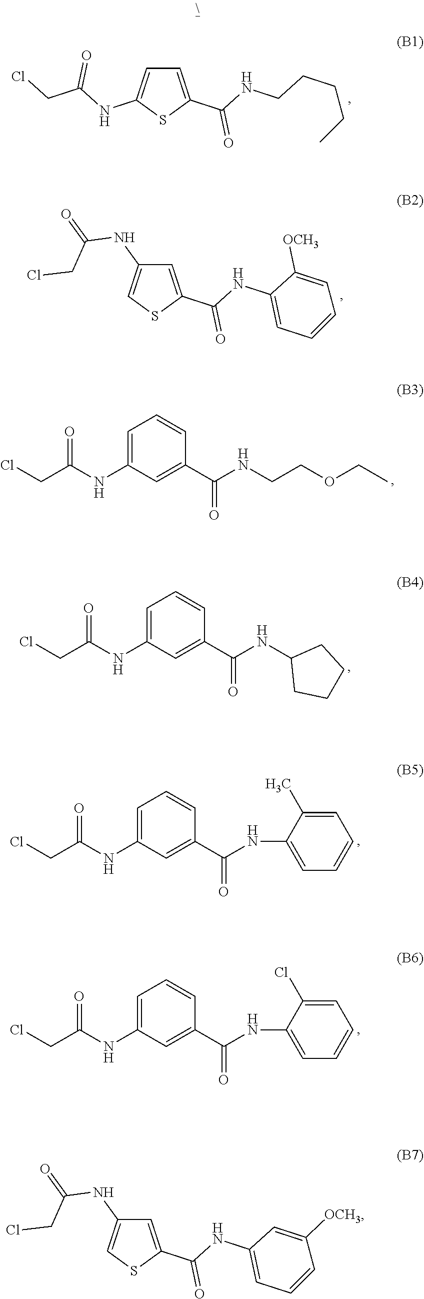

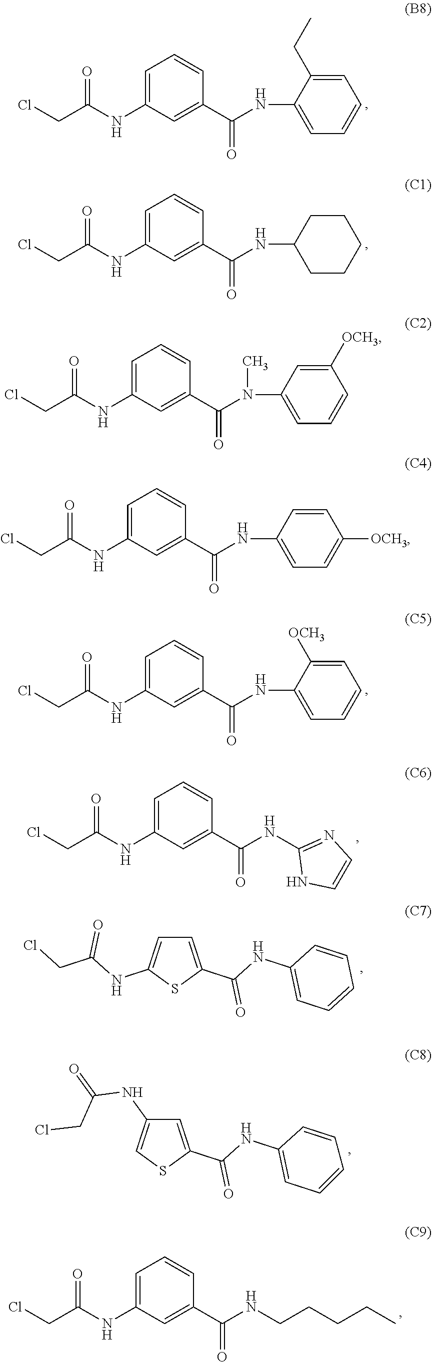

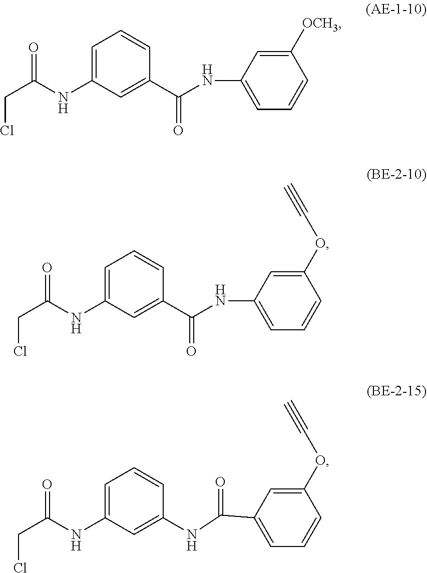 [SMILES: COc1cccc(NC(=O)c2cccc(NC(=O)CCl)c2)c1 ; C#COc1cccc(NC(=O)c2cccc(NC(=O)CCl)c2)c1 ; C#COc1cccc(C(=O)Nc2cccc(NC(=O)CCl)c2)c1]

and a pharmaceutically acceptable excipient.

Embodiments of the disclosure also provide for a method of treating a human subject identified as having or suspected of having COVID-19 or suspected of having a SARS-CoV-2 infection, the method comprising administering to the subject an effective amount of a cytidine triphosphate synthetase 1 (CTPS1) inhibitor effective to reduce interferon regulatory factor 3 (IRF3) deamidation, thereby treating the subject. In some embodiments, the method of treatment comprises administering to a patient a therapeutically effective amount of a composition comprising a CTPS1 inhibitor. The CTPS1 inhibitor may include one or more of the compounds selected from AE-1-10, BE-2-10, BE-2-15, C4, C5, C6, C7, C8, C9, B1, B2, B3, B4, B5, B6, B7, and B8; or one or more of AE-1-10, C4, C5, C6, C7, C8, C9, B1, B2, B4, B5, B7 and B8; or one or more of C5, C9, B1, B2, B4, B5, and B7.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Identification of SARS-CoV-2 proteins that induce IRF3 deamidation to inhibit

IFN induction. (A) NHBE cells were infected with Sendai virus (SeV) (100 HAU/ml) or SARS-CoV-2 (MOI=1). Total RNA was extracted, reverse transcribed and analyzed by real-time PCR with primers specific for IFNB1, ISG15, ISG56, CCLS and Mx1. (B) NHBE cells were transfected with poly(I:C) and RNA isolated from mock- or SARS-CoV-2-infected NHBE cells (MOI=1, 72 hpi). RNA extraction and real-time PCR were performed as in (A). (C) Modulation of IFN-β induction was determined by a promoter activity in 293T cells expressing indicated SARS-CoV-2 proteins, with Sendai virus infection. (D) Inhibition of antiviral gene expression by SARS-CoV-2 proteins in 293T cells infected with SeV was examined by real-time PCR with primers specific for indicated genes. (E) Inhibition of IFN-β induction by selected SARS-CoV-2 proteins was determined by reporter assay of 293T cells expressing TBK-1 and IRF3. (F) Effect of selected SARS-CoV-2 proteins on IRF3 charge status was determined by two-dimensional gel electrophoresis and immunoblotting analyses using lysates of 293T cells transfected with plasmids containing indicated genes. (G) Interactions between exogenous IRF3 and SARS-CoV-2 proteins were analyzed by co-immunoprecipitation in transfected 293T cells. For (F) and (G), Strep indicates SARS-CoV-2 proteins. Error bars indicate standard deviation (SD) of technical triplicates. Statistical significance was calculated using unpaired, two-tailed Student's t-test. *P<0.05; **P<0.01; ***P<0.001. See related FIG. 7.

FIG. 2. CTPS1 inhibits IFN induction. (A) Effect of cellular glutamine amidotransferases (GATs) on IFN induction by Sendai virus infection was determined by luciferase assay using GAT-depleted 293T cells transfected with the IFN-y reporter cocktail: CAD: carbamoyl-phosphate synthetase, aspartate transcarbomylase, and dihydroorotase; CTPS: CTP synthetase; GFPT: glutamine fructose-6-phosphate amidotransferase; GMPS: GMP synthetase; PFAS: phosphoribosylformylglycinamidine synthetase; PPAT: phosphoribosyl pyrophosphate amidotransferase; CPS1: carbamoyl-phosphate synthetase; ASNS: Asparagine synthetase; and NADSYN1: NAD synthetase 1. CTL, control. (B-D) Depletion of CTPS1 was validated by real-time PCR analysis using total RNA extracted from 293T cells infected with lentivirus containing indicated shRNA (B). The mRNA abundance of antiviral genes induced by Sendai virus infection was determined by real-time PCR with primers specific for indicated genes (C). Medium of Sendai virus-infected 293T cells was determined by ELISA for IFN-β and CCLS (D). (E and F)

Knockdown of CTPS1 in THP1 monocytes was determined by immunoblotting using cells with lentivirus containing control (CTL) or CTPS1 shRNA (E). IFN-β and CCLS in the medium of THP1 infected with Sendai virus were determined by ELISA (F). (G) Vesicular stomatitis virus (VSV) replication in control and CTPS1-depleted 293T cells were determined by plaque assay at 10 hours post-infection (MOI=0.01). (H-K) CTPS1 depletion in NHBE cells was determined by immunoblotting (H). Effect of CTPS1 depletion on the expression of cellular antiviral genes (I) and viral genes (J) was determined by real-time PCR analysis of total RNA extracted at 48 h after SARS-CoV-2 infection (MOI=0.1). Medium of NHBE cells infected with SARS-CoV-2 was used for plaque assay to determine infectious viral progeny (K). Error bars indicate SD of technical triplicates. Statistical significance was calculated using unpaired, two-tailed Student's t-test. *P<0.05; **P<0.01; ***P<0.001. See related FIG. 8.

FIG. 3. CTPS1 interacts with and deamidates IRF3. (A) Effect of CTPS1 depletion on IFN induction was determined by luciferase assay in control and CTPS1-depleted 293T cells transfected with the IFN-β reporter cocktail and plasmids containing indicated components of the RIG-I-IFN pathway. (B) Interaction between endogenous IRF3 and CTPS1 was determined by co-immunoprecipitation using 293T cell lysates and immunoblotting analyses. (C and D) Effect of CTPS1 depletion on IRF3 charge status was analyzed by two-dimensional gel electrophoresis and immunoblotting using 293T cells depleted of CTPS1 (C) or expressing the enzyme-deficient CTPS1-ED mutant (D). (E) Effect of several deamidations on IRF3 was determined by luciferase assay using 293T cells transfected with the IFN-β reporter cocktail and plasmids containing TBK-1, wild-type IRF3 or indicated deamidated IRF3 mutants. Whole cell lysates were analyzed by immunoblotting for IRF3 and TBK1 expression. (F and G) Flag-CTPS1, Flag-CTPS1-ED and IRF3-GST proteins were purified from transfected 293T cells by affinity chromatography and analyzed by Coomassie blue staining (F). BSA, bovine serum albumin. In vitro deamidation reactions were analyzed by two-dimensional gel electrophoresis and immunoblotting (G). (H) Effect of CTPS1 on IRF3-N85D was determined by two-dimensional gel electrophoresis and immunoblotting with control (CTL) and CTPS1-depleted 293T cells transfected with a plasmid containing Flag-IRF3-WT or Flag-IRF3-N85D. Error bars indicate SD of technical triplicates. Statistical significance was calculated using unpaired, two-tailed Student's t-test. **P<0.01; ***P<0.001. See related FIG. 9.

FIG. 4. Deamidation impedes IRF3 to activate antiviral immune responses by blocking its DNA binding activity. (A) Expression level of IRF3 was determined by immunoblotting using IRF3/7^{-/-} MEF cells reconstituted with wild-type IRF3, IRF3-N85D and IRF3-N85A. (B and C) The mRNA abundance of antiviral genes was analyzed by real-time PCR in Sendai virus-infected IRF3/7^{-/-} MEF cells reconstituted with wild-type IRF3, IRF3-N85D and IRF3-N85A. (D) IFN-β and (E) CXCL10 in the medium of wild-type IRF3, IRF3-N85D, IRF3-N85A and empty vector reconstituted IRF3/7^{-/-} MEF cells infected with Sendai virus were determined by ELISA. (F) Quantification of mouse Ifnb and Ifna4 promoter sequences were determined by ChIP-qPCR in IRF3-WT and IRF3-N85D reconstituted IRF3/7^{-/-} MEF cells with Sendai virus infection. (G) IRF3-WT and IRF3-N85D proteins were purified from 293T cells by affinity chromatography and analyzed by Coomassie blue staining (left panel). BSA, bovine serum albumin. In vitro IRF3-DNA binding was performed with EMSA by incubating increasing amounts of purified IRF3-WT and IRF3-N85D with DNA probe (2.5 nM) targeting Ifnb promoter (right panel). (H) IRF3-WT, IRF3-N85D, IRF3-N85A and Vector were reconstituted into IRF3/7^{-/-} MEF cells expressing hACE2. Effect of IRF3 and its mutants on the expression of viral genes was determined by real-time PCR of total RNA extracted at 24 h after SARS-CoV-2 infection (MOI=0.01). Medium of the cells infected with SARS-CoV-2 was used for plaque assay to determine infectious viral progeny (H). Error bars indicate SD of technical triplicates. Statistical significance was calculated using unpaired, two-tailed Student's t-test. *P<0.05; **P<0.01; ***P<0.001. See related FIG. 10.

FIG. 5. SARS-CoV-2 promotes CTPS1 enzymatic activity. (A) Interactions between endogenous CTPS1 and SARS-CoV-2 proteins were analyzed by co-immunoprecipitation in transfected 293T cells. Strep indicates SARS-CoV-2 proteins. (B) Effect of SARS-CoV-2 proteins on IRF3 charge status was analyzed by two-dimensional gel electrophoresis and immunoblotting using CTPS1 depleted or control (CTL) 293T cells expressing viral proteins. (C) Effect of SARS-15 CoV-2 proteins on wild-type IRF3 and IRF3-N85A was determined by two-dimensional gel electrophoresis and immunoblotting in IRF3^{-/-} 293T cells expressing GFP-tagged IRF3-WT or IRF3-N85A, along with indicated viral proteins. (D) Effect of SARS-CoV-2 ORF8 and NSP8 on interaction between endogenous CTPS1 and IRF3 was determined by co-immunoprecipitation and immunoblotting analyses in 293T cells expressing ORF8 and Nsp8. (E) Diagram of isotope labeled glutamine metabolism during pyrimidine biosynthesis. (F) Intracellular UTP and CTP traced with [¹⁵N]glutamine were analyzed at 24 h after SARS-CoV-2 (MOI=1) infection by mass spectrometry. M+a indicates the fraction of metabolites labeled with [¹⁵N]glutamine. (G) Effect of SARS-CoV-2 proteins on intracellular CTP and CDP traced with [¹⁵N]glutamine were determined by mass spectrometry in Caco-2 cells infected with lentivirus carrying SARS-CoV-2 ORF7b, ORF8, Nsp8 and control vector. Relative abundance of the metabolites was normalized by cell numbers. (H) Schematic of in vitro CTPS1 enzymatic assay procedure. (I) Effect of SARS-CoV-2 ORF7b and ORF8 on kinetics of CTPS1 activity with respect to UTP, in the presence of 2 mM ATP, 2 mM L-glutamine, 0.1 mM GTP, was determined by in vitro enzymatic assay as described in H, and analyzed by mass spectrometry. Error bars indicate SD of technical triplicates. Statistical significance was calculated using unpaired, two-tailed Student's t-test. *P<0.05; **P<0.01; ***P<0.001. See related FIG. 11.

FIG. 6. CTPS1 inhibitors suppress SARS-CoV-2 replication. (A) Effect of AE-1-10 on SARS-CoV-2 ORF8-induced IRF3 deamidation was analyzed by two-dimensional gel electrophoresis and immunoblotting in ORF8 expressed Caco-2 cells with AE-1-10 (5 μM) treatment. (B) Effect of AE-1-10 on IFN induction by Sendai virus infection was determined by luciferase reporter assay using CTPS1 depleted or control (CTL) 293T cells treated with increasing amount of AE-1-10. (C) Flag-CTPS1 expressed 293T cells were treated with BE-2-10 (5 μM) for 2 h. CTPS1 was purified and subjected to CTPS1-BE-2-10 analysis by in-gel fluorescence imaging and Coomassie blue staining. (D and E) Effect of AE-1-10 on intracellular CTP and CDP traced with [¹⁵N]glutamine was determined by mass spectrometry using SARS-CoV-2 ORF8 expressed Caco-2 cells (F) or SARS-CoV-2 infected Caco-2 cells (G) treated with increasing amount of AE-1-10. (G-I) Caco-2 cells were treated with AE-1-10 and infected with SARS-CoV-2 (MOI=0.1). The mRNA abundance of antiviral genes was determined by real-time PCR at 48 h after SARS-CoV-2 infection (H). Effect of AE-1-10 on the expression of viral genes (I) and infectious viral progeny (J) was determined at 72 h after SARS-CoV-2 infection by real-time PCR analysis of total RNA and plaque assay of the medium, respectively. (K) Effect of AE-1-10 and its derivatives on SARS-CoV-2 replication was determined by plaque assay at 72 h post-infection (MOI=0.1) in medium of Caco-2 cells. Error bars indicate SD of technical triplicates. Statistical significance was calculated using unpaired, two-tailed Student's t-test. *P<0.05; **P<0.01; ***P<0.001. See related FIG. 12.

FIG. 7. SARS-CoV-2 inhibits IFN induction. (A and B) Calu-3 and Caco-2 cells were infected with Sendai virus (SeV) (100 HAU/ml) or SARS-CoV-2 (MOI=1). Total RNA was extracted, reverse transcribed and analyzed by real-time PCR with primers specific for IFNB1, ISG15, ISG56, CCLS and Mx1. (C) Diagram of the RIG-I-IFN pathway that can be triggered by RNA virus infection. (D) Effect of SARS-CoV-2 proteins on IFN-β induction was determined by luciferase reporter assay in 293T cells transfected with IFN-β reporter cocktail, plasmids containing increasing dose of SARS-CoV-2 viral proteins and indicated components of the RIG-I-IFN pathway. Error bars indicate SD of technical triplicates. Statistical significance was calculated using unpaired, two-tailed Student's t-test. *P<0.05; **P<0.01; ***P<0.001.

FIG. 8. CTPS1 inhibits IFN induction. (A) Knockdown of CTPS1 in 293T cells was determined by immunoblotting using cells infected with lentivirus containing control (CTL) or CTPS1 shRNA. (B) The mRNA abundance of antiviral genes induced by Sendai virus infection was determined by real-time PCR at 12 h post-infection using CTPS1 depleted and control (CTL) THP1 cells. (C) Knockdown of CTPS1 in Caco-2 cells was determined by immunoblotting using cells infected with lentivirus containing control (CTL) or CTPS1 shRNA. (D) Effect of CTPS1 depletion on the expression of cellular antiviral genes was determined by real-time PCR at 24 h post SARS-CoV-2 (MOI=0.5) infection in CTPS1 depleted and control Caco-2 cells. (E and F) CTPS1 depleted and control Caco-2 cells were infected with SARS-CoV-2 (MOI 0.1) for 72 h. Effect of CTPS1 depletion on viral genes expression was determined by real-time PCR analysis of total RNA. Viral titer in the medium was measured by plaque assay. Error bars indicate SD of technical triplicates. Statistical significance was calculated using unpaired, two-tailed Student's t-test. *P<0.05; **P<0.01; ***P<0.001.

FIG. 9. CTPS1 deamidates IRF3. (A) IFN-β promoter activity was determined by report assay in 293T cells transfected with IFN-β reporter plasmid cocktail and expression plasmids containing increasing amount of IRF3-5D. (B) Modulation of IFN-β induction was determined by a promoter activity in 293T cells expressing CTPS1-WT and CTPS1-ED, with Sendai virus infection. (C) Effect of 6-diazo-5-oxo-L-norleucine (DON) on IFN-β induction was determined by luciferase reporter assay in CTPS1 depleted and control (CTL) 293T cells, with Sendai virus infection. (D) Interactions between IRF3 and CTPS1, CTPS2 were analyzed by co-immunoprecipitation and immunoblotting in 293T cells transfected with plasmids containing GST-IRF3, and Flag-CTPS1 or Flag-CTPS2. (E) Effect of several deamidations on IRF3 was determined by luciferase assay using 293T cells transfected with the ISRE reporter cocktail and plasmids containing increasing amount of wild-type IRF3 or indicated deamidated IRF3 mutants. (F) IFN-β promoter activity was determined in 293T cells transfected with IFN-β reporter plasmid cocktail and expression plasmids containing TBK1, and increasing amount of IRF3-WT and mutants. Expression levels of the components were analyzed by immunoblotting. (G) Effect of CTPS1 on IRF3-N85A and IRF3-N85Q was determined by two-dimensional gel electrophoresis and immunoblotting with control (CTL) and CTPS1-depleted 293T cells transfected with a plasmid containing IRF3-N85A or IRF3-N85Q. Error bars indicate SD of technical triplicates. Statistical significance was calculated using unpaired, two-tailed Student's t-test. *P<0.05; **P<0.01; ***P<0.001.

FIG. 10. Deamidation impedes IRF3 to activate antiviral immune responses by blocking its DNA binding activity. (A) IRF3-WT and IRF3-N85D reconstituted IRF3/7^{-/-} MEF cells were infected with SeV for 12 h. Phosphorylation and dimerization of IRF3 were resolved by SDS-PAGE or native PAGE with the whole cell lysates, and followed by immunoblotting analysis with indicated antibodies. (B and C) IRF3-WT and IRF3-N85D reconstituted IRF3/7^{-/-} MEF cells were infected with or without Sendai virus. Subcellular localization of IRF3-WT and IRF3-N85D were analyzed at 24 h post-infection by immunofluorescence using a confocal microscope. Scale bars, 5 μm (B). Cells with IRF3-WT or IRF3-N85D nuclear localization were counted at indicated time points after Sendai infection. Error bars indicate SD. NS, no significance (C). (D and E) IRF3-WT and IRF3-N85D reconstituted IRF3/7^{-/-} MEF cells were infected with VSV-GFP (MOI 0.01) for 10 h. GFP positive cells were recorded with fluorescence microscopy (D). Viral replication in medium was determined by plaque assay (E). (F) IRF3/7^{-/-} MEF cells were infected with lentivirus containing Flag-tagged human ACE2, selected with hygromycin, then reconstituted with Vector, IRF3-WT, IRF3-N85D and IRF3-N85A. Expression levels of ACE2 and IRF3 were analyzed by immunoblotting. (G) RF3-WT, IRF3-N85D, IRF3-N85A and vector reconstituted IRF3/7^{-/-} MEF cells expressing human ACE2 were infected with SARS-CoV-2 (MOI=0.1). The mRNA abundance of Ifnb, Isg15, Isg56 and Cxc110 were analyzed by real-time PCR at indicated time points post-infection. Error bars indicate SD of technical triplicates. Statistical significance was calculated using unpaired, two-tailed Student's t-test. NS, no significance; *P<0.05; **P<0.01; ***P<0.001.

FIG. 11. SARS-CoV-2 promotes CTP synthesis by targeting CTPS1. (A) Interaction between CTPS1 and SARS-CoV-2 proteins were determined by co-immunoprecipitation and immunoblotting in 293T cells transfected with plasmid containing Flag-CTPS1 and indicated strep-tagged SARS-CoV-2 proteins. Strep indicates SARS-CoV-2 proteins (B) Effect of the selected SARS-CoV-2 proteins on IRF3 deamidation were analyzed by two-dimensional gel electrophoresis and immunoblotting using Caco-2 cells infected with lentivirus carrying SARS-CoV-2 ORF7b, ORF8, Nsp8 and Vector. (C) Effect of SARS-CoV-2 proteins on intracellular UTP and CTP traced with [¹⁵N]glutamine were determined by mass spectrometry in LoVo cells infected with lentivirus carrying SARS-CoV-2 ORF7b, ORF8, Nsp8 and control vector. Relative abundance of the metabolites was normalized by cell numbers. Error bars indicate SD of technical triplicates. Statistical significance was calculated using unpaired, two-tailed Student's t-test. *P<0.05; **P<0.01; ***P<0.001.

FIG. 12. CTPS1 inhibitors suppress SARS-CoV-2 replication. (A) Effect of Q6ca, AE-1-10, BE-2-10 or BE-2-15 on IRF3 deamidation was analyzed by two-dimensional gel electrophoresis and immunoblotting in A549 cells expressing SARS-CoV-2 Nsp8. (B) The mRNA abundance of IFNB1 was determined by real-time PCR analysis using total RNA extracted from 293T cells pre-treated with Q6ca, BE-2-10, BE-2-15, AE-1-10 and DON for 2 h, followed with SeV infection for 9 h. (C) Effect of AE-1-10 on NF-κB promoter activity was determined by luciferase reporter assay using 293T cells transfected with NF-κB reporter plasmid cocktail, followed with AE-1-10 treatment at indicated concentration. (D) Effect of AE-1-10 on intracellular CTP and CDP traced with [15N]glutamine was determined by mass spectrometry using SARS-CoV-2 ORF8 expressed LoVo cells treated with increasing amount of AE-1-10. (E-G) NHBE cells with treated with AE-1-10 at indicated concentration. The mRNA abundance of IFNB1 and ISG15 was determined by real-time PCR analysis at 24 h after SARS-CoV-2 (MOI=0.5) infection (E). Effect of AE-1-10 on expressions of viral genes was analyzed by real-time PCR of total RNA extracted at 48 h after SARS-CoV-2 infection (MOI=0.1) (F). Medium of NHBE cells infected with SARS-CoV-2 was used for plaque assay to determine viral replication (G). (H) Caco-2 cells were treated with the AE-1-10 and its derivatives at indicated concentration for each 24 h. Cell viability was determined by trypan blue staining at 72 h. (I) Effect of AE-1-10 and its derivatives on IFN-β promoter activity was determined by luciferase reporter assay in AE-1-10 and its derivatives-treated 293T cells with SeV infection. (J) RNA abundance of viral genes (Nsp1, N and E) in Caco-2 cells treated with C4, C5, C7, C8, C9 and AE-1-10 was determined by real-time PCR analysis at 72 h after SARS-CoV-2 (MOI=0.1) infection. Error bars indicate SD of technical triplicates. Statistical significance was calculated using unpaired, two-tailed Student's t-test. NS, no significance; *P<0.05; **P<0.01; ***P<0.001.

FIG. 13. The antiviral activity and cytotoxicity of CTPS1 inhibitors in distinct cell lines. (A-D) NHBE (A), Caco-2 (B), Vero E6 (C) and Calu-3 (D) cells were treated with the indicated compounds and infected with SARS-CoV-2 (MOI=0.1). Viral titer in the medium was determined by plaque assay. Effects of these compounds on cell viability were determined by XTT assay and plotted. IC₅₀, IC₉₀ and CC₅₀ were calculated. Error bars indicate SD of technical triplicates.

FIG. 14. A CTPS1 inhibitor protects mice from SARS-CoV-2 infection. (A) Schematic of SARS-CoV-2 infection in the AAV-hACE2 mouse model. (B-D) AAV-hACE2 C57BL/6J mice were intranasally infected with 1.5×10⁵ PFU of SARS-CoV-2, and treated with 35 mg/kg of C9 (n=9) or vehicle (n=9) daily by intraperitoneal injection. Mice were euthanized on 4 dpi, and lung tissues were harvested. Cellular antiviral gene expression (B) and RNA abundance of SARS-CoV-2 (C) were analyzed by real-time PCR. Viral titer in the lung homogenates was determined by plaque assay in Vero E6 cells. Dotted gray line indicates the limit of detection of viral titer. (D). (E-I) K18-hACE2 transgenic mice (n=5 for each group) were pre-treated with C9 or vehicle control for 1 day, and infected with 1×10⁴ PFU SARS-CoV-2 for 3 days. Compound treatment was performed daily. mRNA of innate immune genes (E) and indicated viral RNA (F) in the lung were analyzed by real-time PCR. Viral titer in the lung was determined by plaque assay using Vero E6 cells. Dotted gray line indicates the limit of detection of viral titer (G). Immunofluorescence staining was performed on the frozen lung tissues with an antibody against SARS-CoV-2 nucleocapsid protein (NP). Scale bar in top panels is 100 μm; scale bar in lower panels is 20 μm (H). Lung tissues were harvested, processed and sectioned for H&E staining. AL, alveolus; BR, bronchioles; black arrows indicate inflammatory infiltrates. Scale bar in top panels is 200 μm; scale bar in lower panels is 100 μm (I). Error bars indicate SD. Statistical significance was calculated using unpaired, two-tailed Student's t-test. *P<0.05; **P<0.01; ***P<0.001.

FIG. 15. Antiviral activity of derivatives (B series) of CTPS1 inhibitors. (A and B) Caco-2 cells were pretreated with 2 μM of C9, B1-B8 (DMSO as control) for 2 h. Cells were then infected with SARS-CoV-2 (MOI 0.1) and compounds were added daily. SARS-CoV-2 RNA abundance was determined at 72 h after viral infection by real time-PCR analysis using total RNA from the cells. (C) Viral titer in the medium was determined by plaque assay. (D) Caco-2 cells were seeded into 96-well plates for 24 h. The cells were treated with the indicated concentrations of compounds daily. Cell viability was determined with a XTT assay kit, and normalized with

DMSO control group at 72 h post treatment. Error bars indicate SD. Statistical significance was calculated using unpaired, two-tailed Student's t-test. **P<0.01; ***P<0.001.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14 th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject. The compounds and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms, or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at one carbon atom, or two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, P-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

### Embodiments of the Invention

This disclosure provides for certain enzymatic inhibitor compounds, compositions comprising certain enzymatic inhibitor compounds, and methods of use, generally comprising formula I, II, or III:

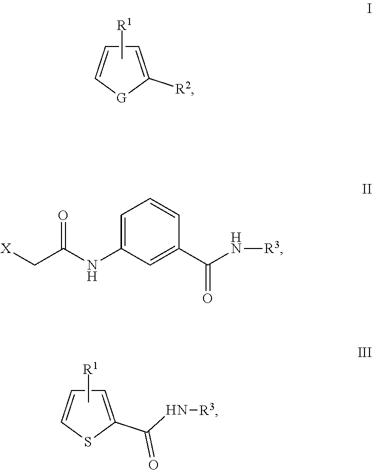

wherein, G is

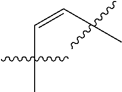 [SMILES: */C=C\*]

or S; R¹ is —NH(C═O)CH₂X wherein X is a leaving group; R² is —(C═O)NHR³; or R² is —(C═O)NR^{a}R³ or —NR^{a}(C═O)R³, wherein R^{a} is H or —(C₁-C₆)alkyl. R³ is —(C₁-C₆)alkyl, —(C₃-C₆)cycloalkyl, phenyl-R⁴, or 5- or 6-membered heteroaryl; and R⁴ is —O(C₁-C₆)alkyl, —(C₁-C₆)alkyl, halo, or H; wherein R¹ is in the beta-position relative to R² and each (C₁-C₆)alkyl moiety is independently saturated or unsaturated. Optionally, each (C₁-C₆)alkyl moiety is interrupted by a heteroatom.

In some embodiments, the enzymatic inhibitor compound is one or more pharmaceutically acceptable salts of a compound of formula I, formula II, or formula III.

In some embodiments, the R¹ is at the 4-position. In some embodiments, the R¹ is at the 5-position. In some embodiments, X is halo, N₃, methanesulfonate (OMs), or p-toluenesulfonate (OTs). In some embodiments, X is chloro.

In some embodiments, R³ is pentyl, cyclopentyl, cyclohexyl, phenyl-O(propargyl), phenyl-OCH₃, phenyl-H, or imidazolyl. In some embodiments, R³ is —(CH₂)ₘO(CH₂)ₙCH₃ wherein m is 2-6 and n is 0-6. In some embodiments, when R³ is —(C₁-C₆)alkyl, —(C₁-C₆)alkyl is interrupted by a heteroatom or one or more heteroatoms.

In some embodiments, R³ is phenyl-R⁴ and R⁴ is ortho-OCH₃, meta-OCH₃, para-OCH₃. In some embodiments, R⁴ is in the ortho-, meta-, or para-position. In some embodiments, R⁴ is halo, chloro, —(C₁-C₆)alkyl, methyl, ethyl, propyl, ethoxy, propargyl.

In some embodiments, the enzymatic inhibitor or pharmaceutically acceptable salt thereof is one or more of:

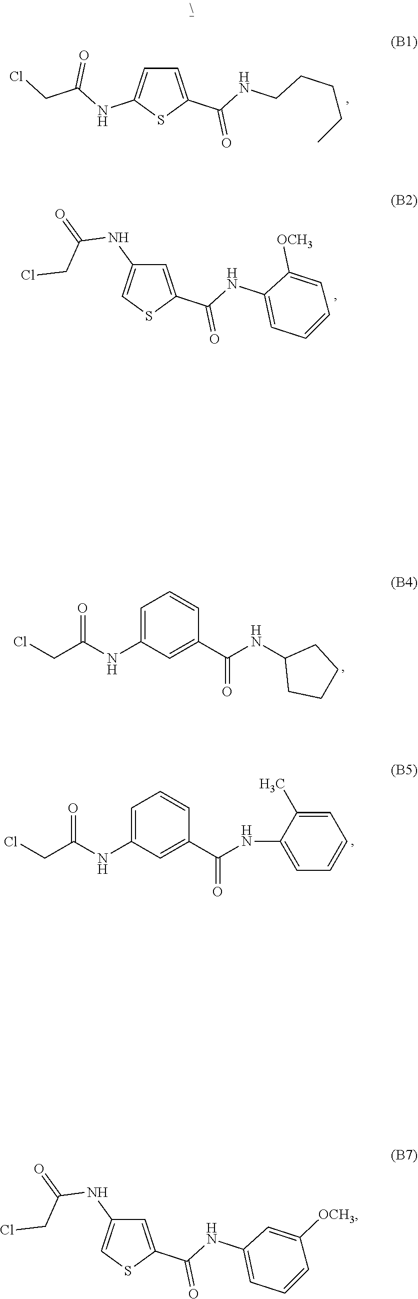 [SMILES: CCCCCNC(=O)c1ccc(NC(=O)CCl)s1 ; COc1ccccc1NC(=O)c1cc(NC(=O)CCl)cs1 ; O=C(CCl)Nc1cccc(C(=O)NC2CCCC2)c1 ; Cc1ccccc1NC(=O)c1cccc(NC(=O)CCl)c1 ; COc1cccc(NC(=O)c2cc(NC(=O)CCl)cs2)c1]

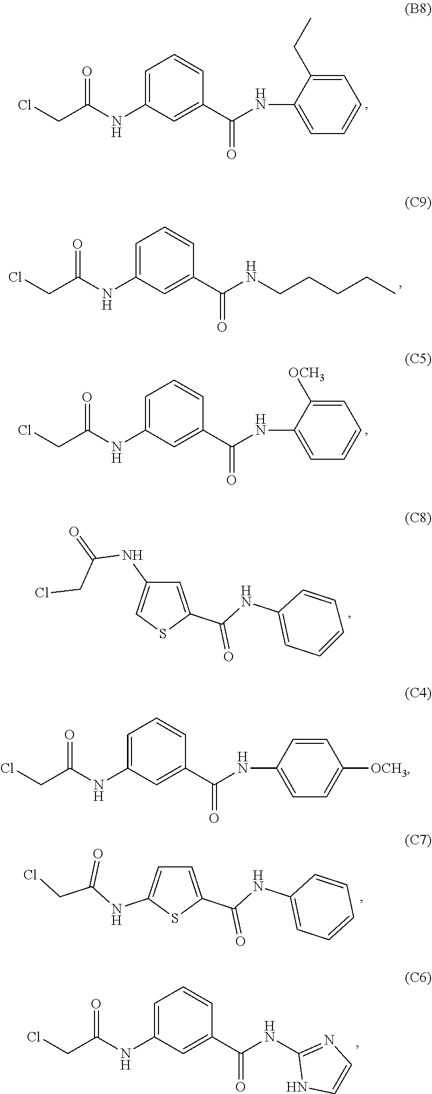

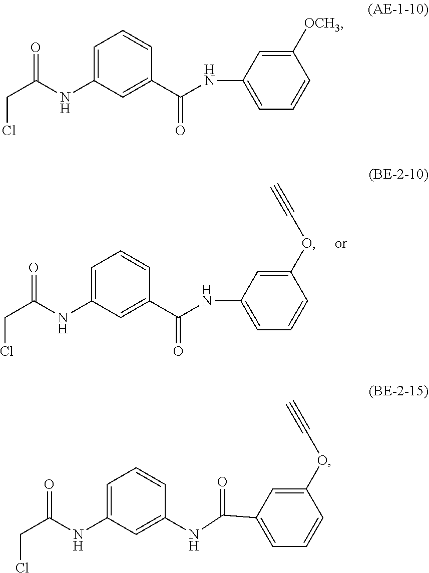 [SMILES: COc1cccc(NC(=O)c2cccc(NC(=O)CCl)c2)c1 ; C#COc1cccc(NC(=O)c2cccc(NC(=O)CCl)c2)c1 ; C#COc1cccc(C(=O)Nc2cccc(NC(=O)CCl)c2)c1]

or a pharmaceutically acceptable salt thereof.

This disclosure provides for certain enzymatic inhibitor compounds generally comprising formula IV:

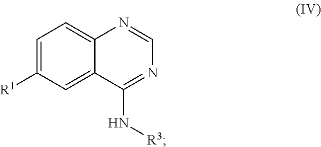

wherein the definitions of R¹ and R³ are that same as for formula I, II or III. In some embodiments, the enzymatic inhibitor compound is:

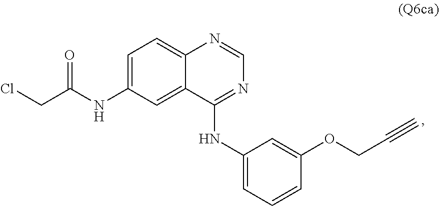 [SMILES: C#CCOc1cccc(Nc2ncnc3ccc(NC(=O)CCl)cc23)c1]

or a pharmaceutically acceptable salt thereof.

In certain preferred embodiments, the enzymatic inhibitor compound is one or more of:

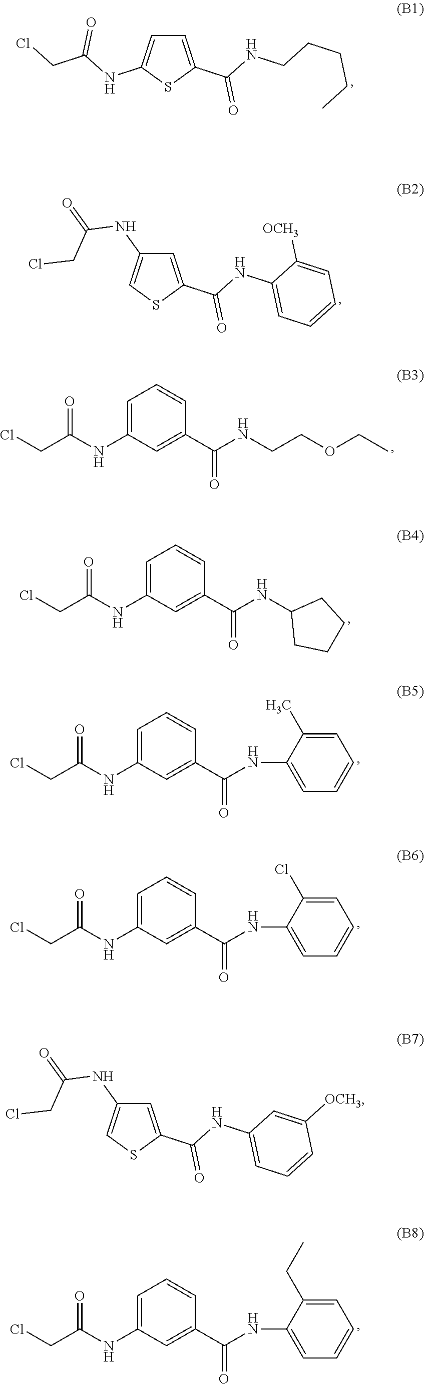

or a pharmaceutically acceptable salt thereof. In certain preferred embodiments, the enzymatic inhibitor compound is one or more of:

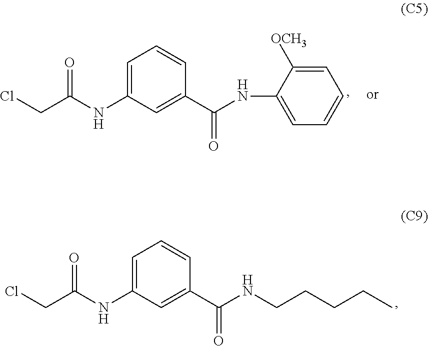 [SMILES: COc1ccccc1NC(=O)c1cccc(NC(=O)CCl)c1 ; CCCCCNC(=O)c1cccc(NC(=O)CCl)c1]

or a pharmaceutically acceptable salt thereof.

In some embodiments, the enzymatic compounds maybe formulated into a pharmaceutical composition comprising the enzymatic inhibitor compound and a pharmaceutically acceptable excipient.

In some embodiments the enzymatic inhibitor compound reduces, eliminates, or otherwise inhibits the enzymatic activity of cellular glutamine amidotransferases. In some embodiments, the enzymatic inhibitor reduces or eliminates enzymatic activity of cytidine triphosphate synthetase 1 (CTPS1). In some embodiments, the CTPS1 inhibitor is one or more of AE-1-10, BE-2-10, BE-2-15, C4, C5, C6, C7, C8, C9, B1, B2, B3, B4, B5, B6, B7, and B8. In other embodiments, the CTPS1 inhibitor is one or more of AE-1-10, C4, C5, C6, C7, C8, C9, B1, B2, B4, B5, B7 and B8. In some embodiments, the CTPS1 inhibitor is one or more of C5, C9, B1, B2, B4, B5, and B7.

Embodiments of the disclosure also provide for a method of antiviral treatment comprising administering to a subject in need thereof a therapeutically effective amount of a compound as described herein or a pharmaceutically acceptable salt thereof, or administering a composition comprising a therapeutically effective amount of a compound as described herein or a pharmaceutically acceptable salt thereof, thereby inhibiting replication of a virus that has infected the subject. Preferably, the compound is one or more AE-1-10, BE-2-10, BE-2-15, C4, C5, C6, C7, C8, C9, B1, B2, B3, B4, B5, B6, B7, and B8. In other embodiments, the compound is one or more of AE-1-10, C4, C5, C6, C7, C8, C9, B1, B2, B4, B5, B7 and B8.

In some embodiments, the antiviral treatment comprises treatment for an infection caused by a coronavirus such as Severe Acute Respiratory Syndrome Coronavirus (SARS-CoV), Severe Acute Respiratory Syndrome Coronavirus 2 (SARS-CoV-2), Middle East Respiratory Syndrome Coronavirus (MERS-CoV), and variants thereof. In some embodiments, coronavirus infection is SARS-CoV-2, the causative agent of COVID-19 disease.

In some embodiments, a method for antiviral treatment comprises administering to a subject having or suspected of having a viral infection the enzymatic inhibitor compound or the pharmaceutically acceptable salt thereof, in combination with one or more additional therapeutic agents. In some embodiments, the enzymatic inhibitor or pharmaceutically acceptable salt thereof and the one or more additional therapeutic agents are administered simultaneously or sequentially.

In some embodiments, a method for antiviral treatment comprises administering to a subject having or suspected of having a viral infection a composition comprising the enzymatic inhibitor compound or the pharmaceutically acceptable salt thereof, in combination with one or more additional therapeutic agents. In some embodiments, the composition comprising the enzymatic inhibitor or pharmaceutically acceptable salt thereof and the one or more additional therapeutic agents are administered simultaneously or sequentially.

It is contemplated that any convenient type of therapeutic agent may be employed, where examples of therapeutic agent types include, but are not limited to, small molecules, nucleic acids, specific binding member for a coronavirus protein, such as, but not limited to, antibodies, aptamers, peptides, etc.

In some embodiments, the therapeutic agent is an antibody that binds to a coronavirus protein. An antibody that specifically binds to a coronavirus protein can be polyclonal or monoclonal antibody or fragments that are sufficient to bind a coronavirus protein. The antibody fragments can be, for example, monomeric Fab fragments, monomeric Fab′ fragments, or dimeric F(ab)′2 fragments, single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In some embodiments, the therapeutic agent is a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the active agent includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, may be used to target a protein-coding as well as non-protein-coding genes. In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc. In some embodiments, the gene silencing molecules target the human CTSP1 gene described, for example, in the NCBI database accession number NG_034208.1, or an mRNA transcript thereof such as described in, but not limited to, the NCBI database accession numbers NM_001905.4, NM_001301237.2, AI190299.1 AK130549.1 AK225899.1 AK297386.1 AK299122.1 AK303797.1, AK304817.1, BC009408.1, BQ642049.1, DA639348.1, DC313620.1, and X52142.1. In some embodiments, the targeted gene is a homolog of human CTSP1.

In some embodiments, the additional therapeutic agent is one or more of paxlovid, sotrovimab, remdesivir, molnupiravir, tocilizumab, bamlanivimab plus etesevimab, and casirivimab plus imdevimab, imatinib mesylate, nilotinib hydrochlorde, dasatinib interferons, ribavirin, adefovir, tenofovir, acyclovir, brivudin, cidofovir, fomivirsen, foscarnet, ganciclovir, penciclovir, amantadine, rimantadine, zanamivir, chloroquine phosphate, hydroxychloroquine sulfate, mefloquine, amodiaquine dihydrochloride dihydrate, and gemcitabine hydrochloride.

The disclosure also provides for a method of treating a human subject identified as having or suspected of having COVID-19, the method comprising administering to the subject an effective amount of a cytidine triphosphate synthetase 1 (CTPS1) inhibitor, thereby treating the subject.

In some embodiments, a method of treating a human subject identified as having or suspected of having COVID-19 comprises administering to the subject an effective amount of a cytidine triphosphate synthetase 1 (CTPS1) inhibitor effective to reduce interferon regulatory factor 3 (IRF3) deamidation, thereby treating the subject. In some embodiments, the CTPS1 inhibitor or pharmaceutically acceptable salt thereof is formulated into a composition.

In some embodiments, the CTPS1 inhibitor is a compound according to formula I, II, or II. In other embodiments, the CTPS1 inhibitor is one or more of compounds of AE-1-10, BE-2-10, BE-2-15, C4, C5, C6, C7, C8, C9, B1, B2, B3, B4, B5, B6, B7, and B8. In other embodiments, the CTPS1 inhibitor is one or more of AE-1-10, C4, C5, C6, C7, C8, C9, B1, B2, B4, B5, B7 and B8. In some embodiments, he CTPS1 inhibitor is one or more of C5, C9, B1, B2, B4, B5, and B7.

### Results and Discussion

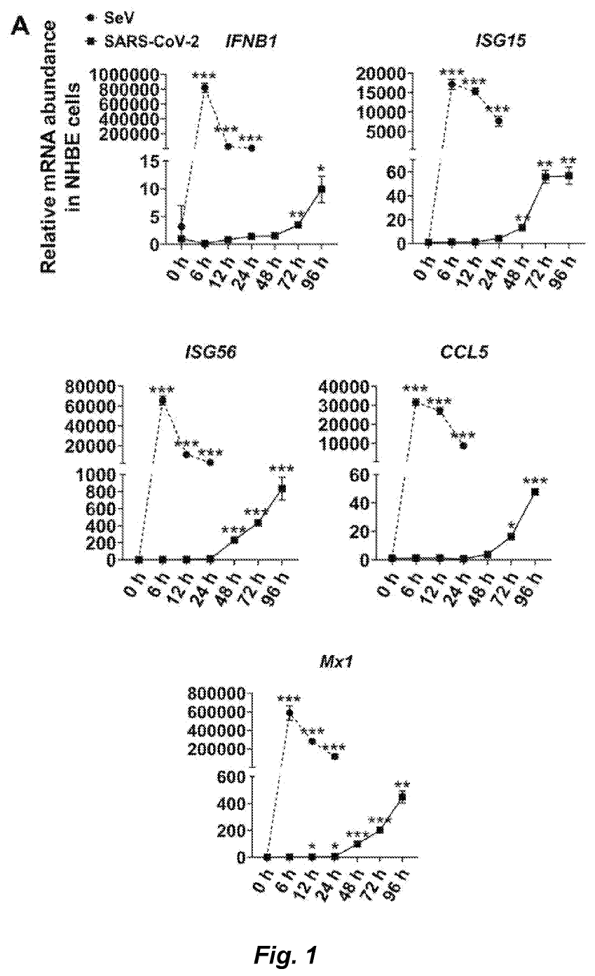

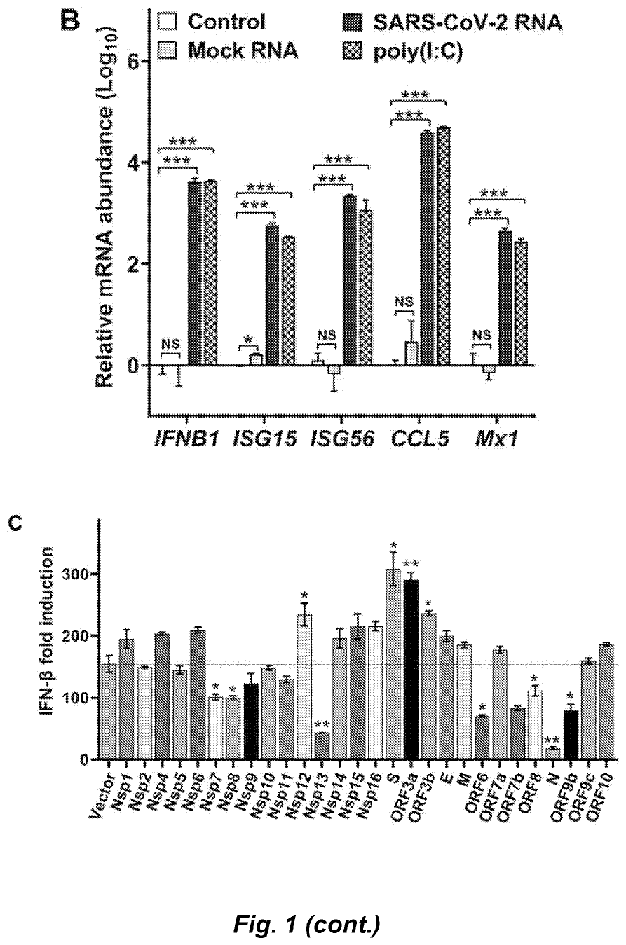

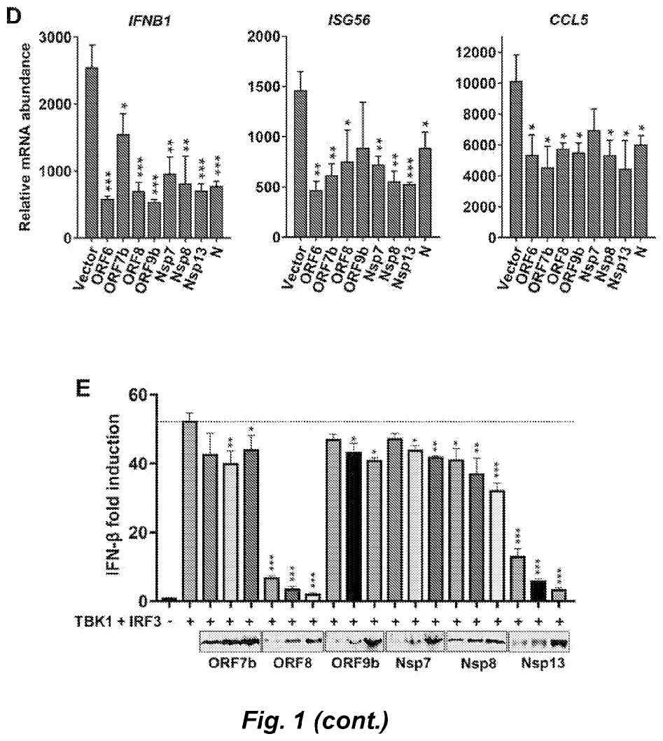

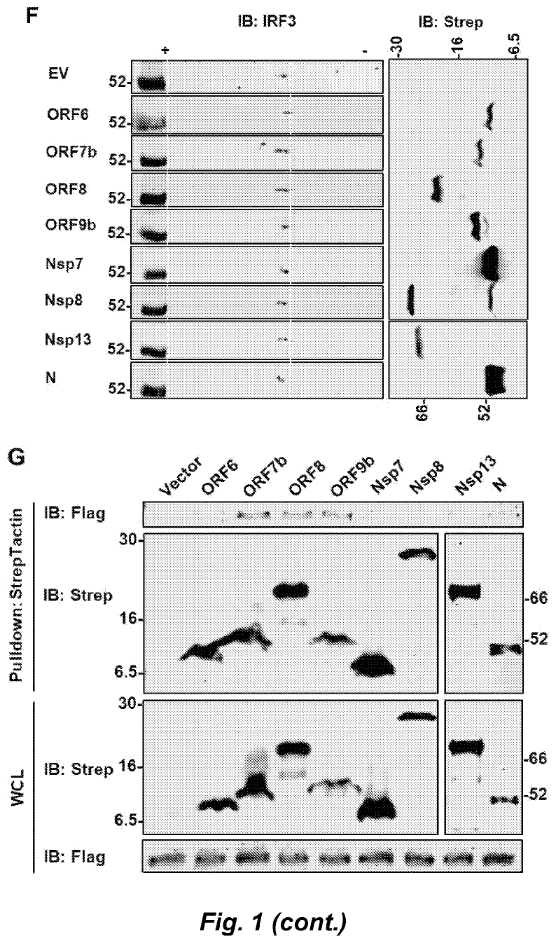

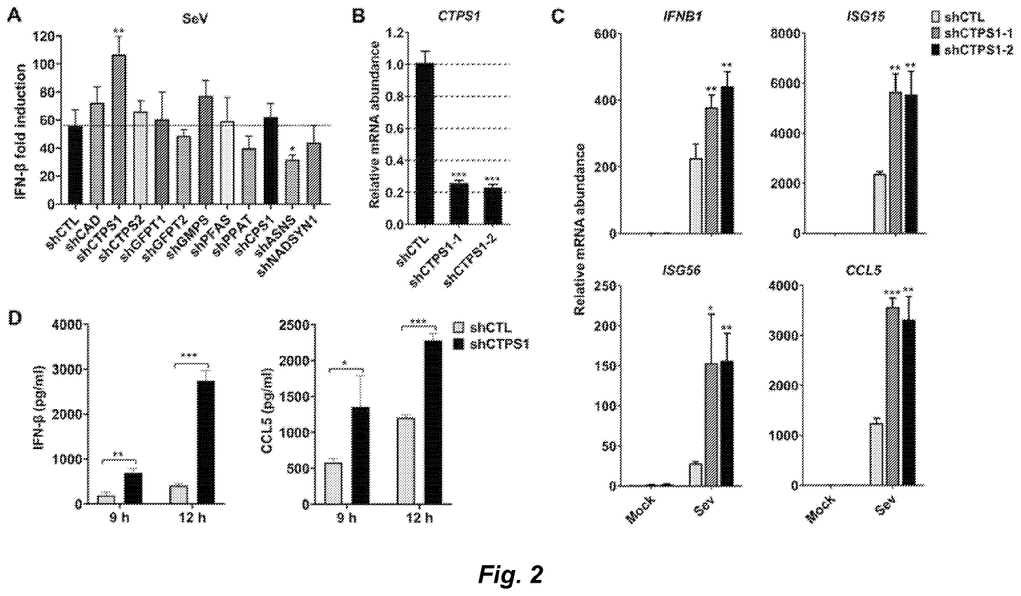

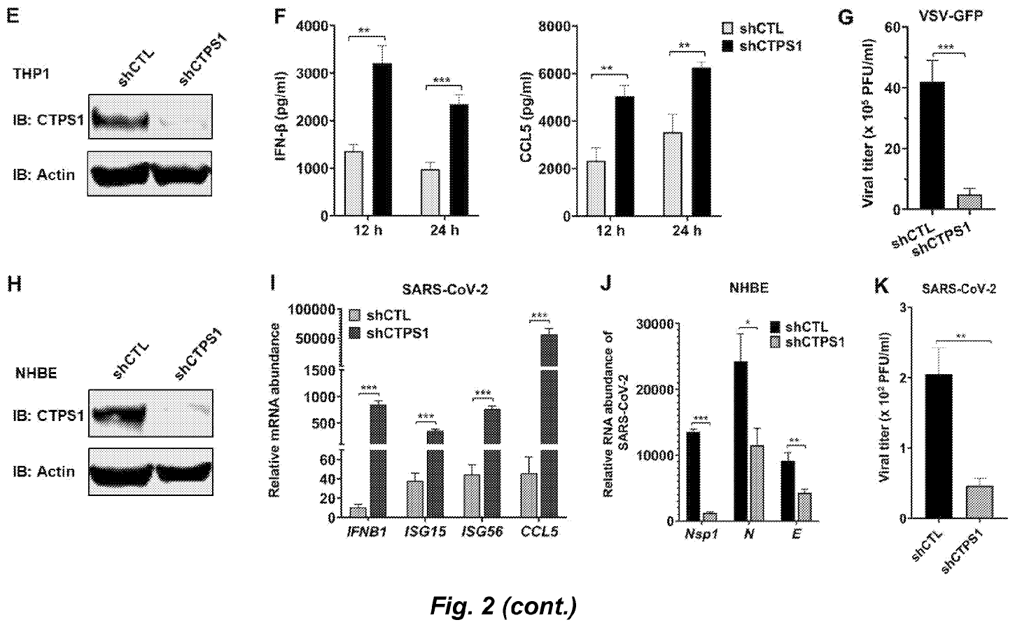

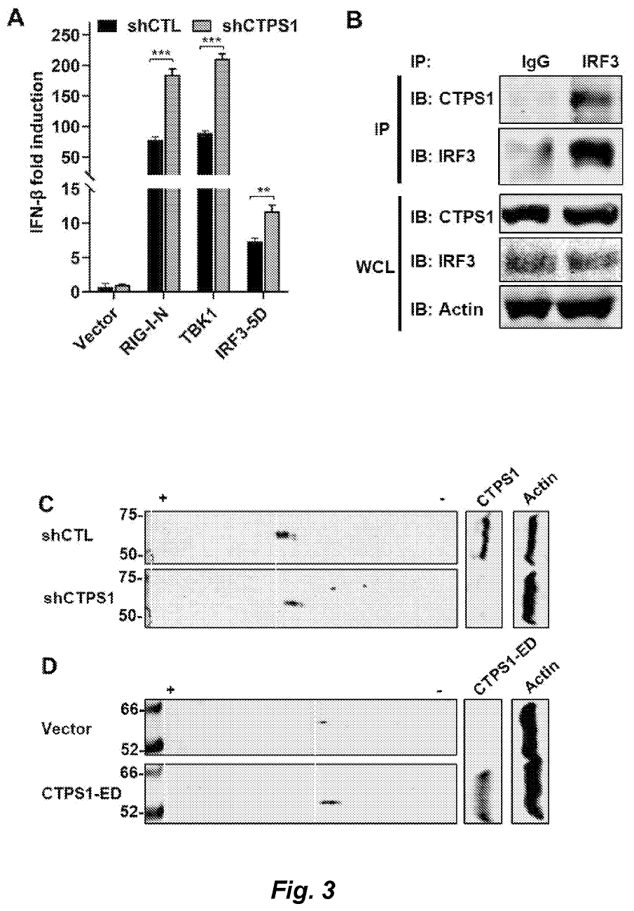

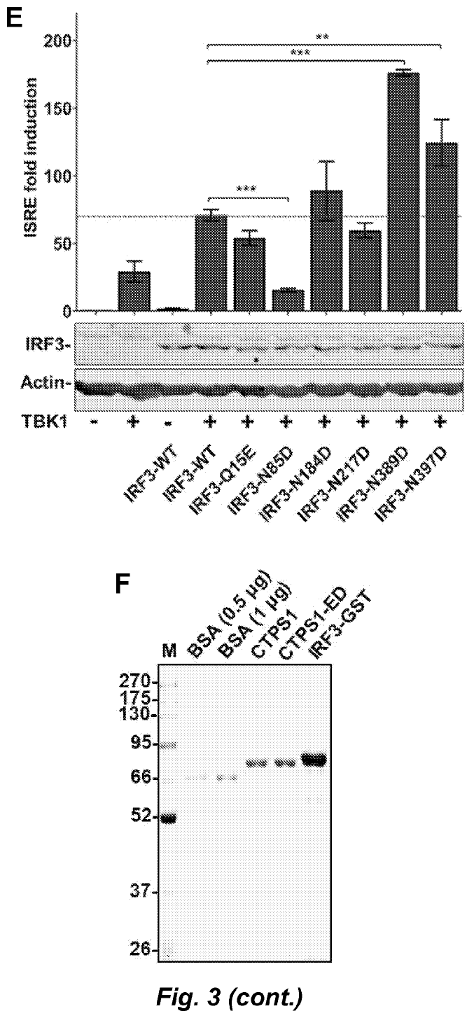

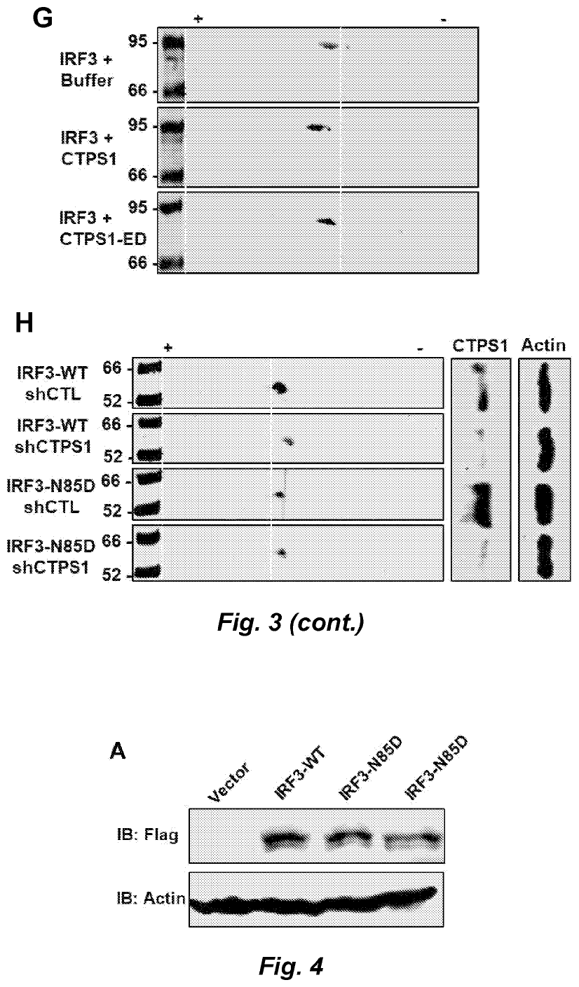

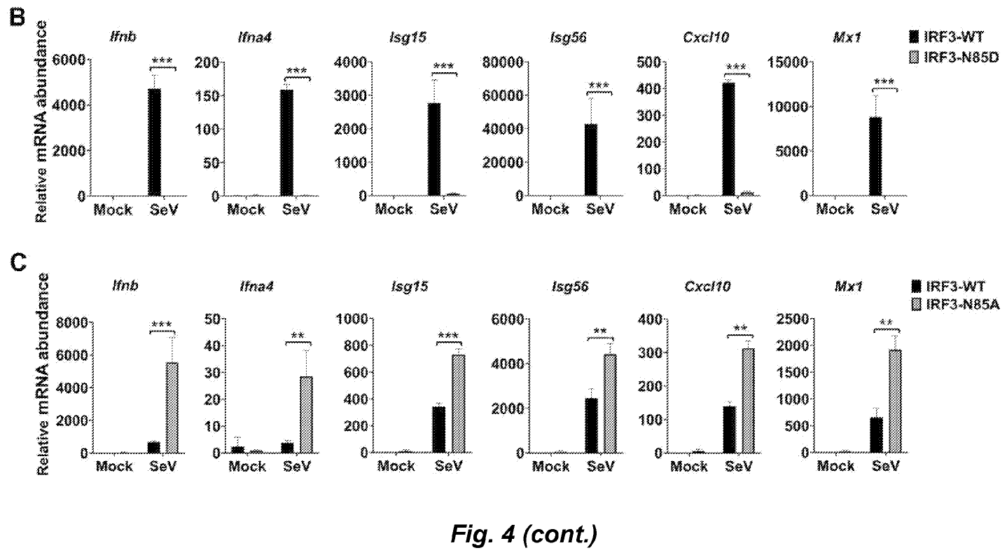

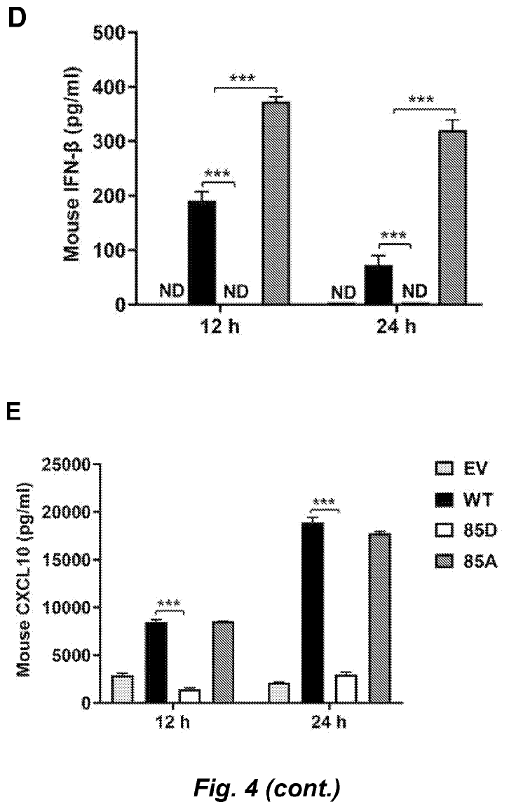

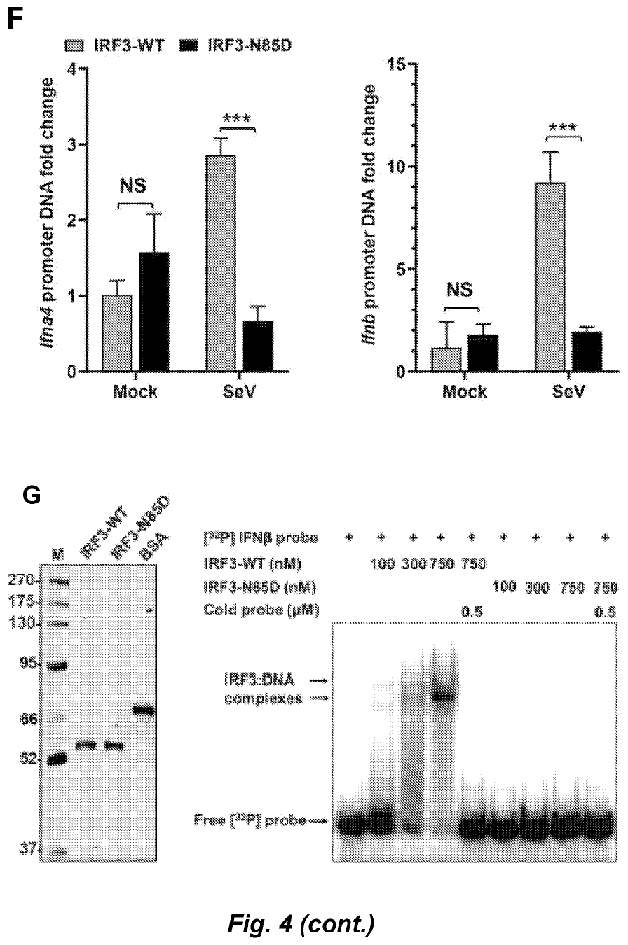

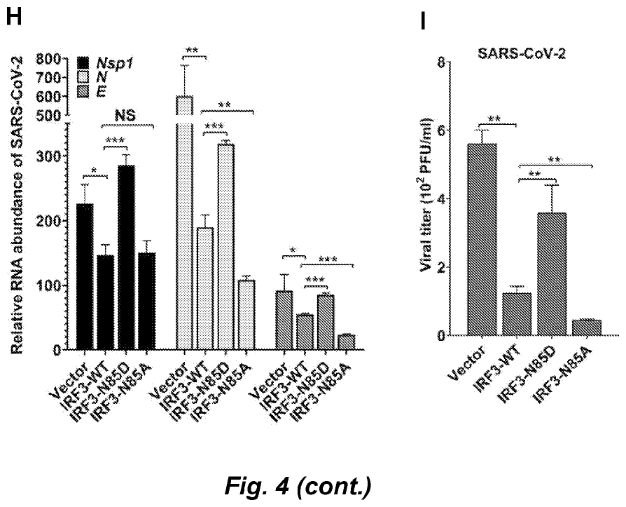

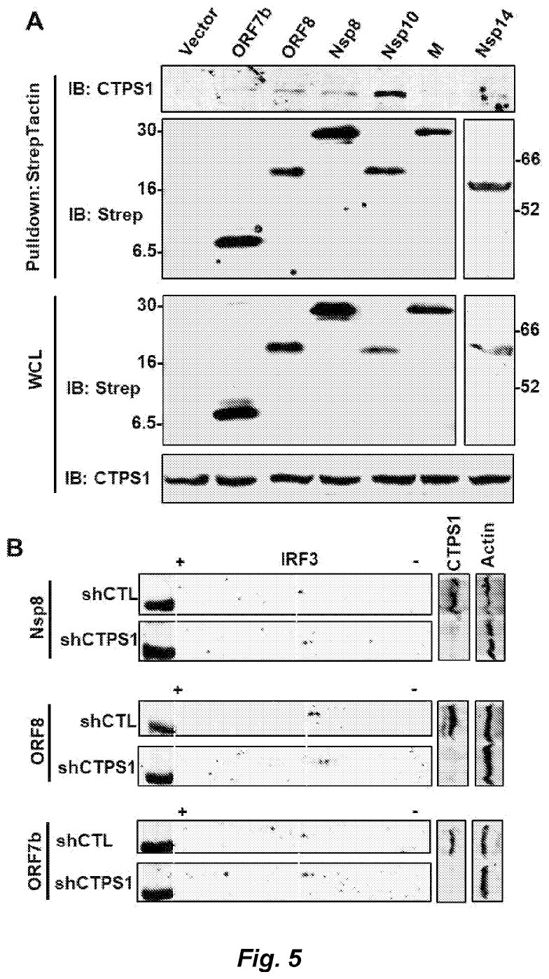

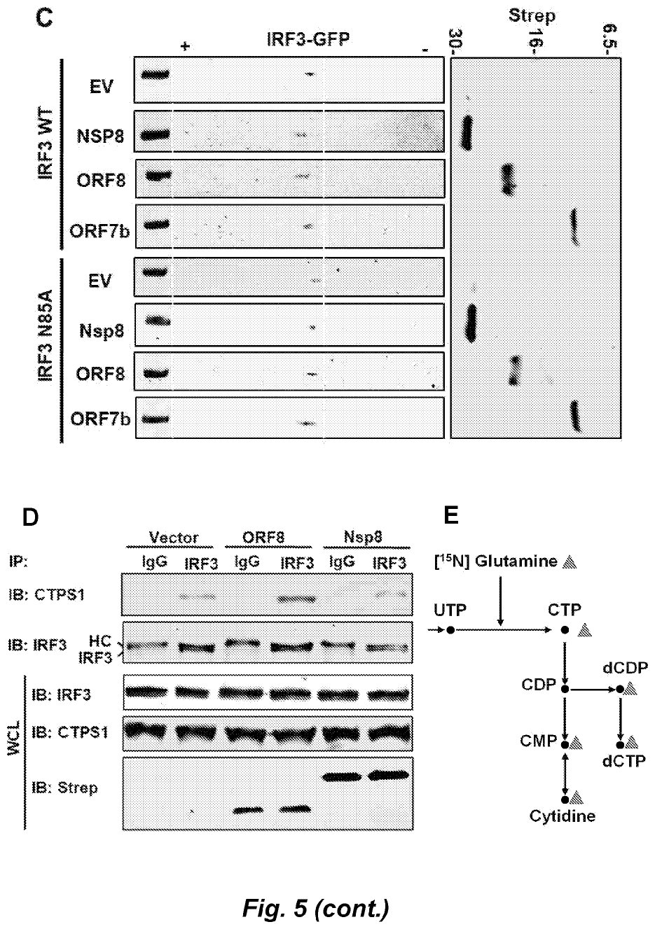

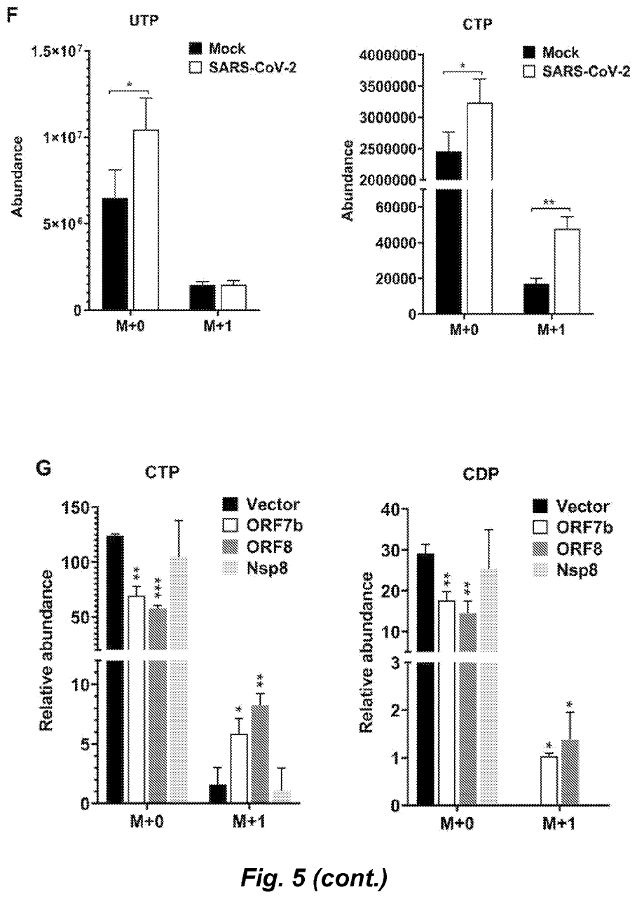

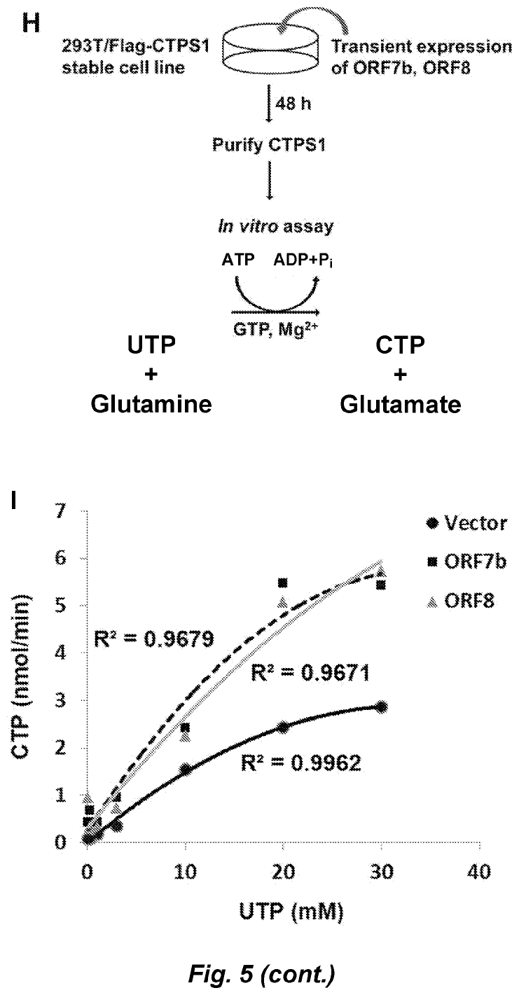

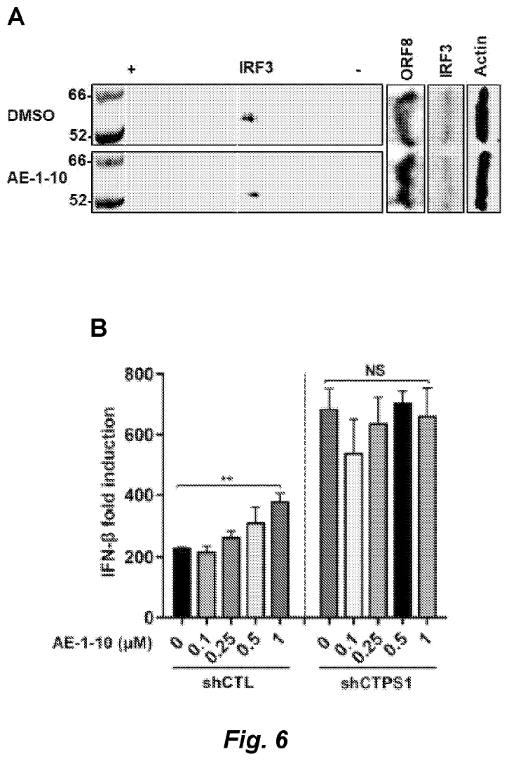

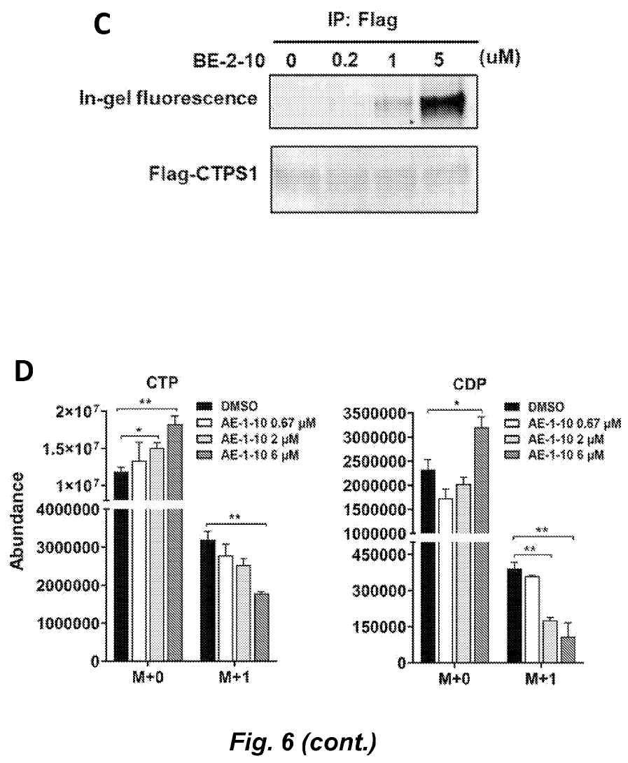

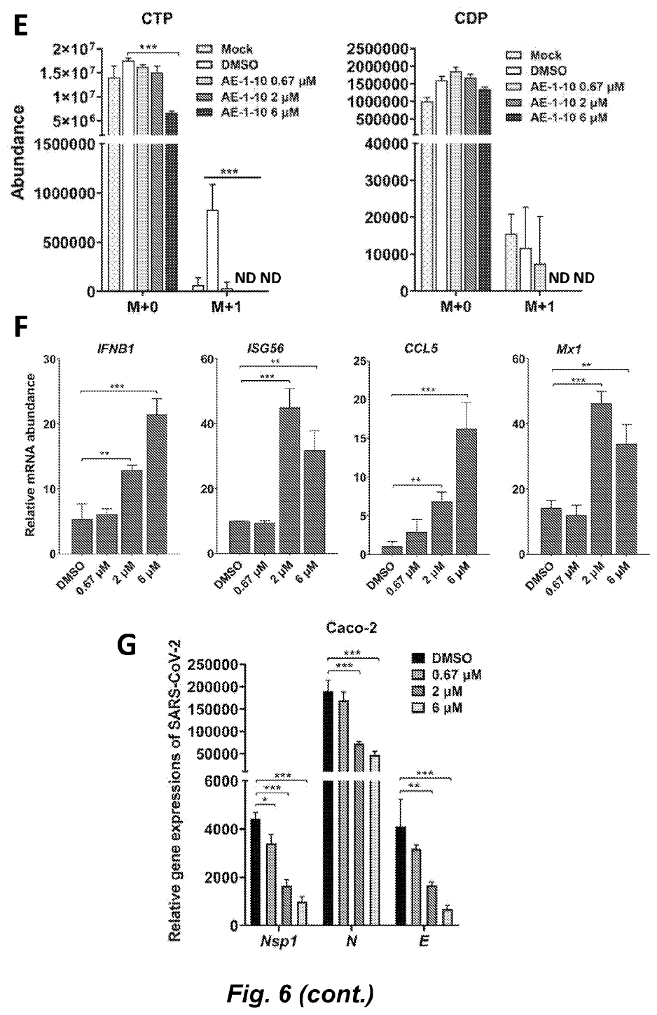

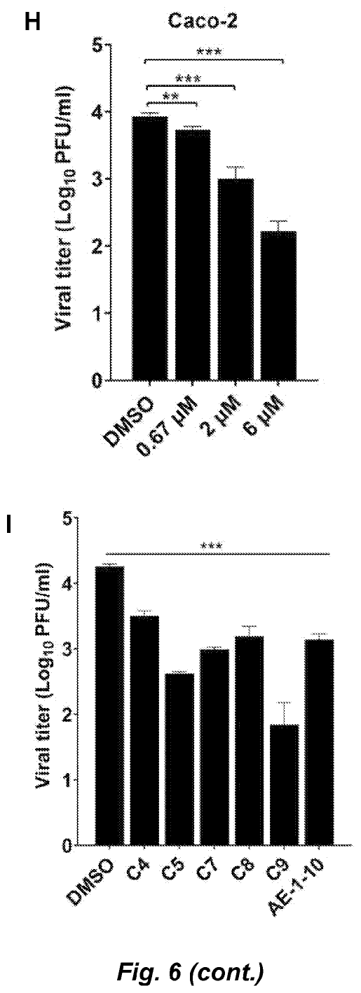

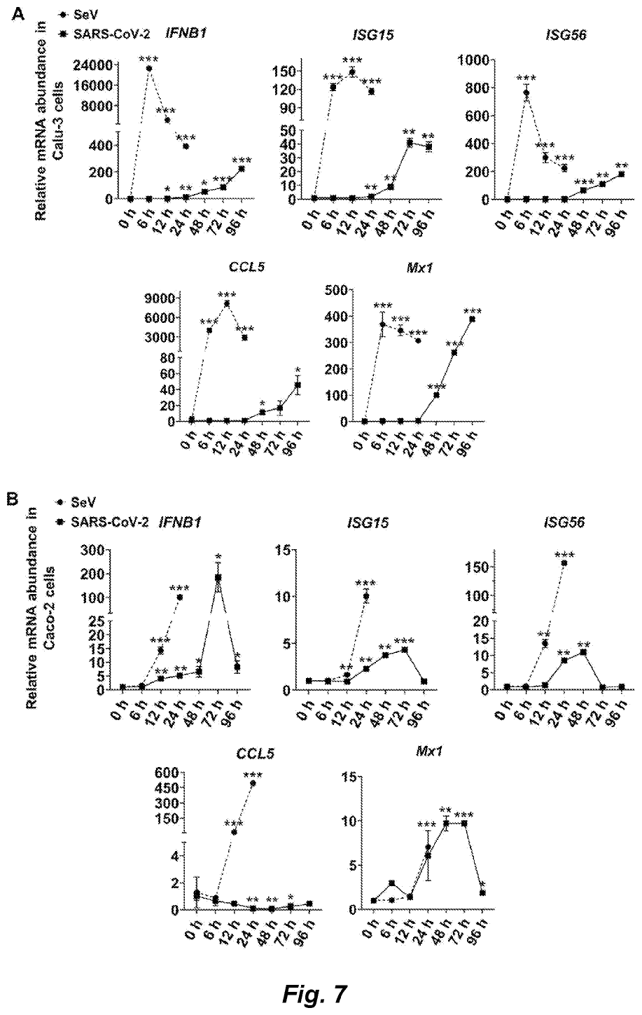

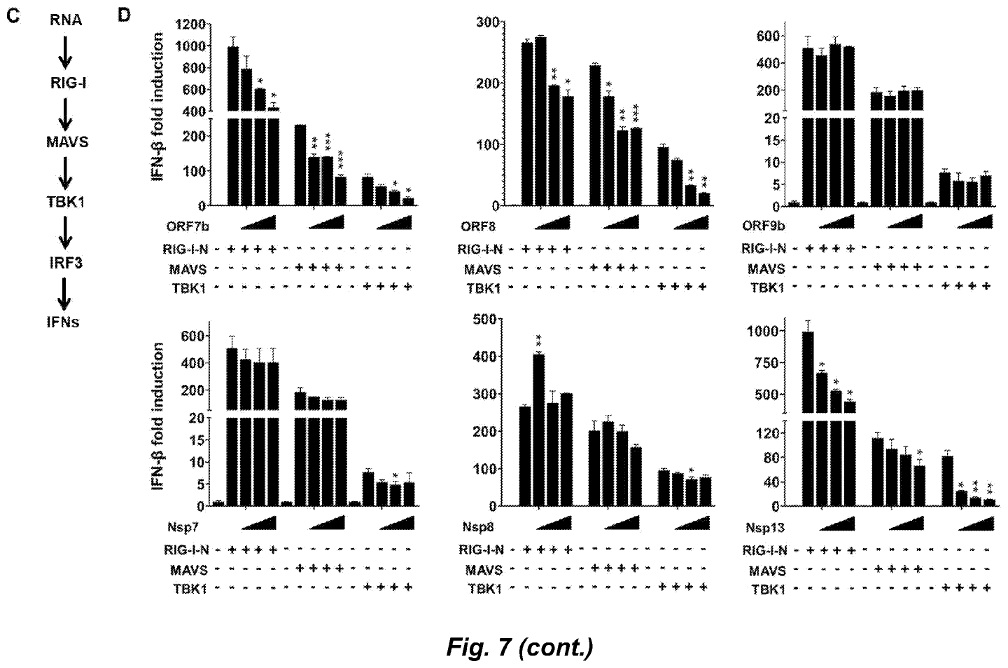

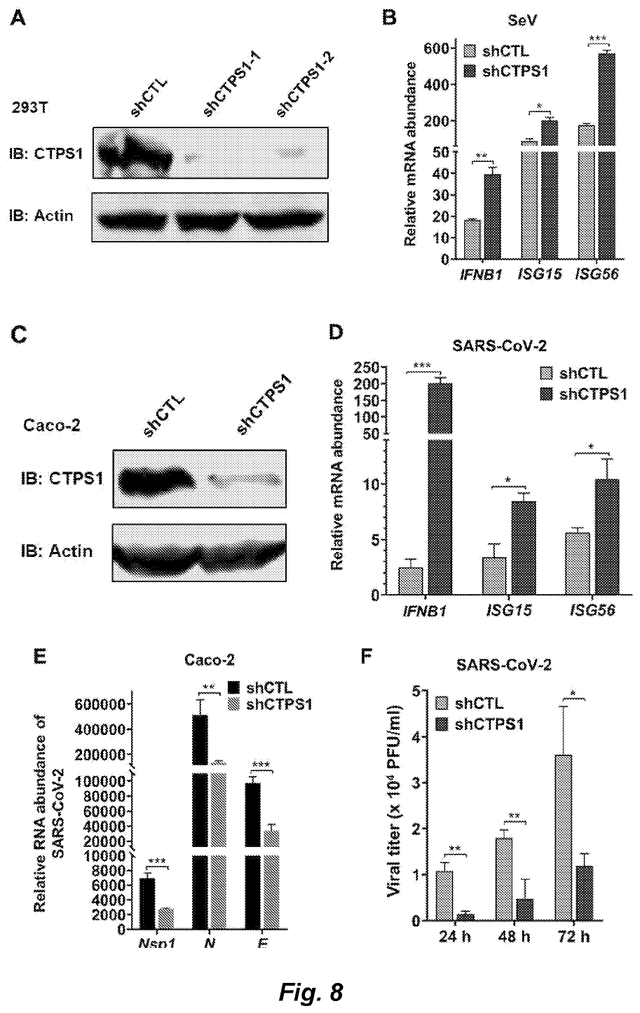

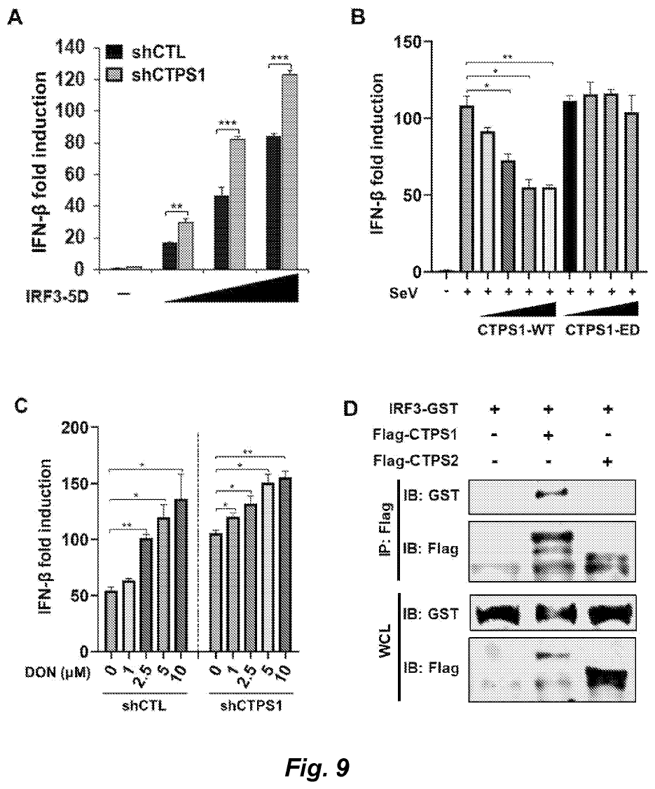

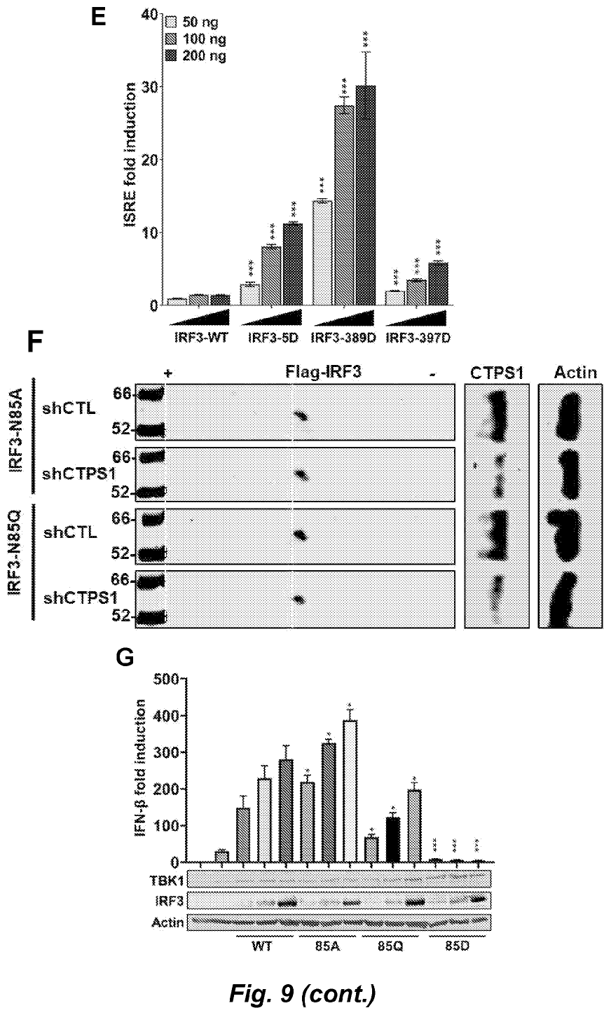

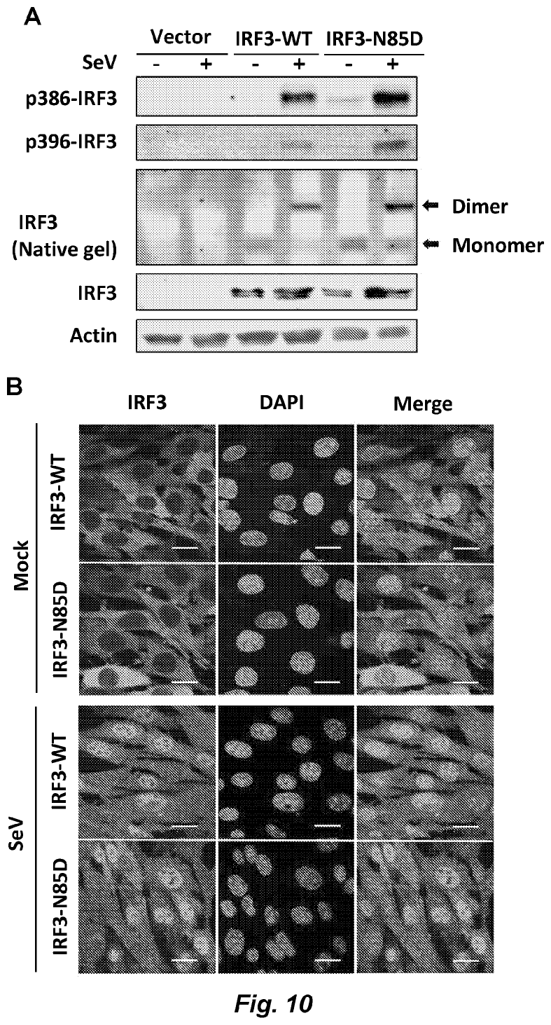

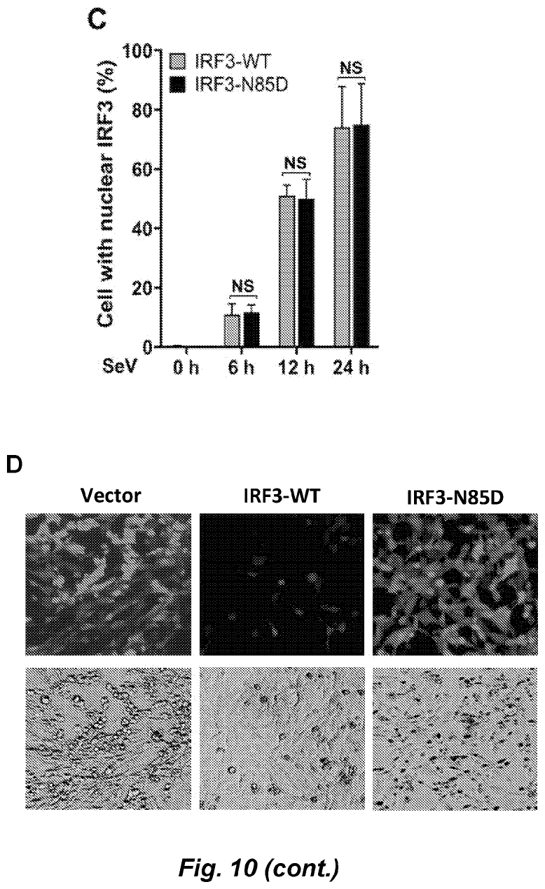

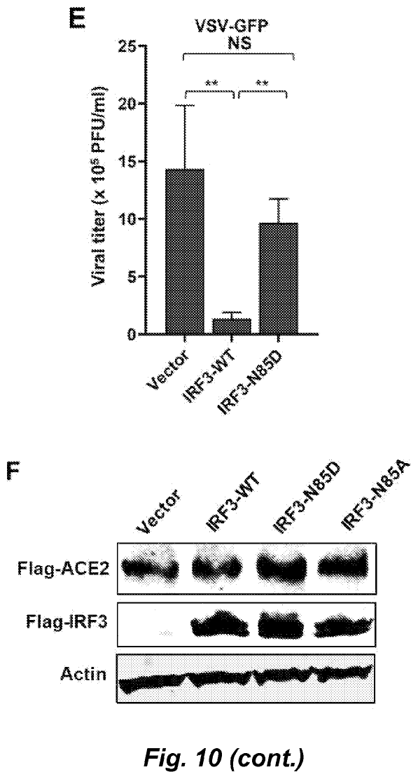

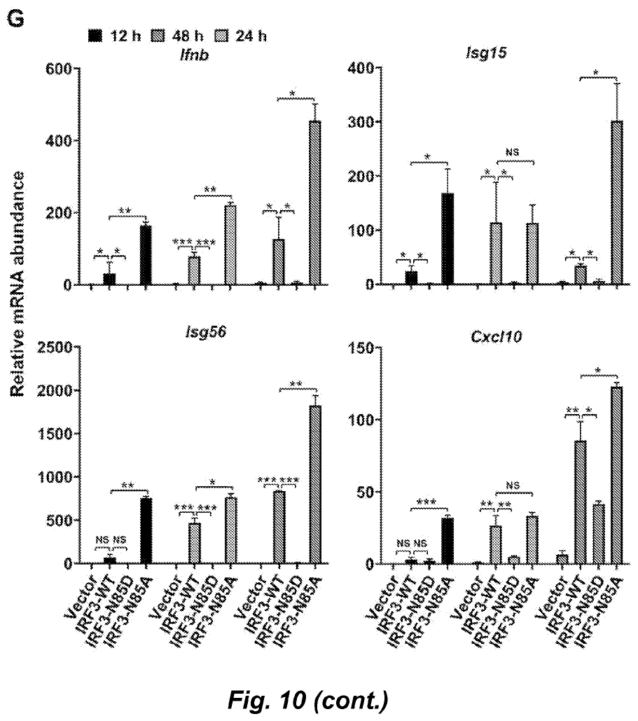

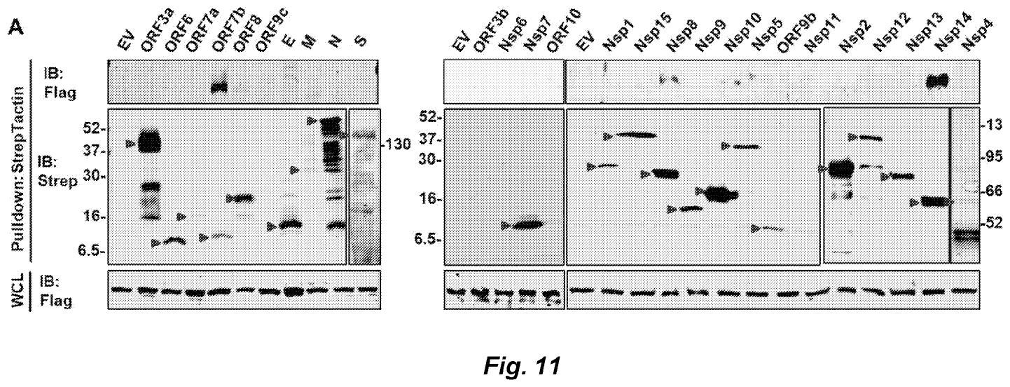

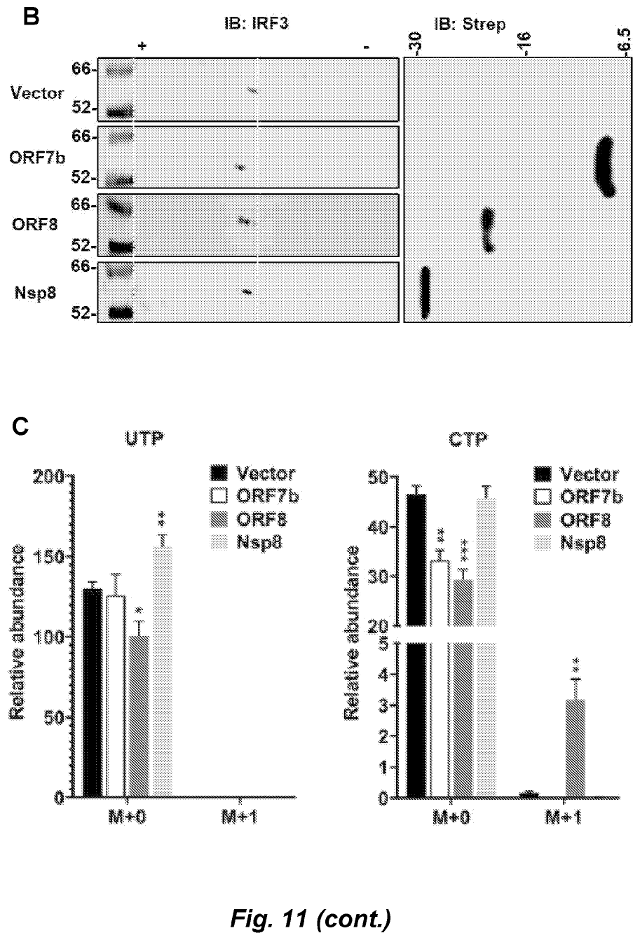

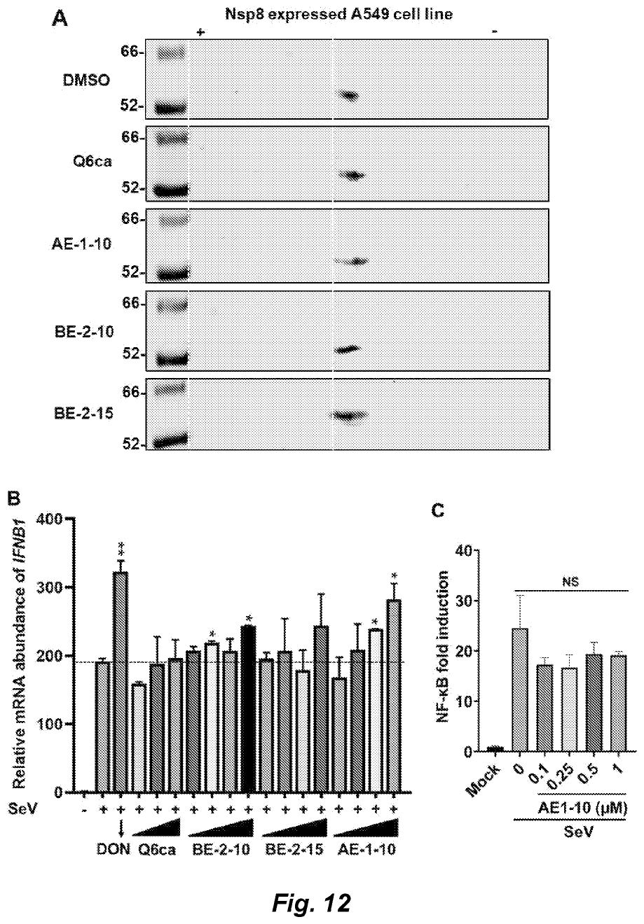

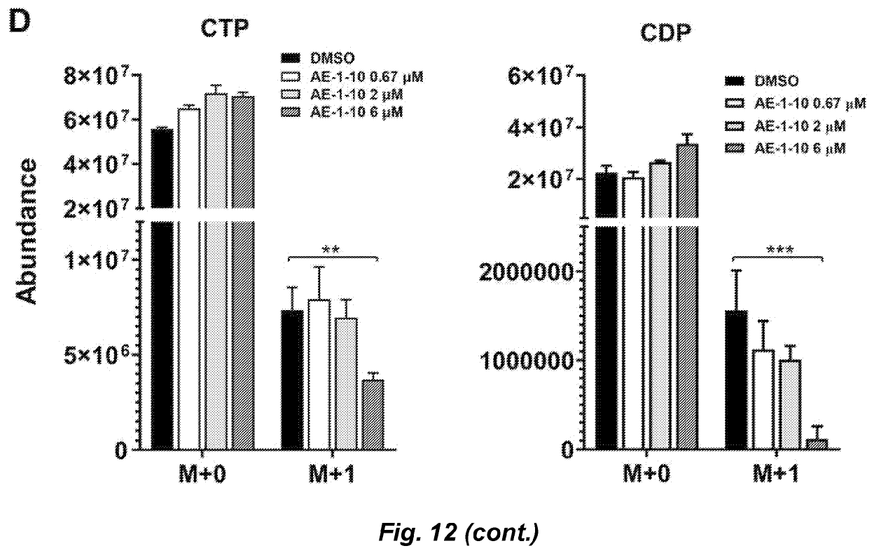

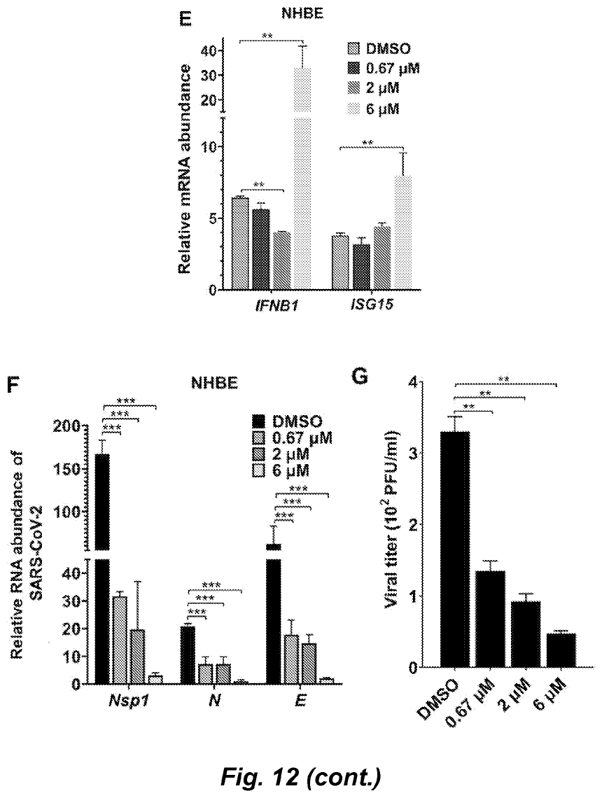

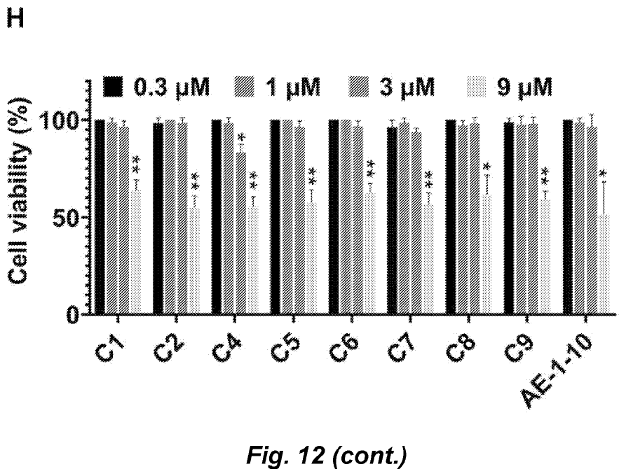

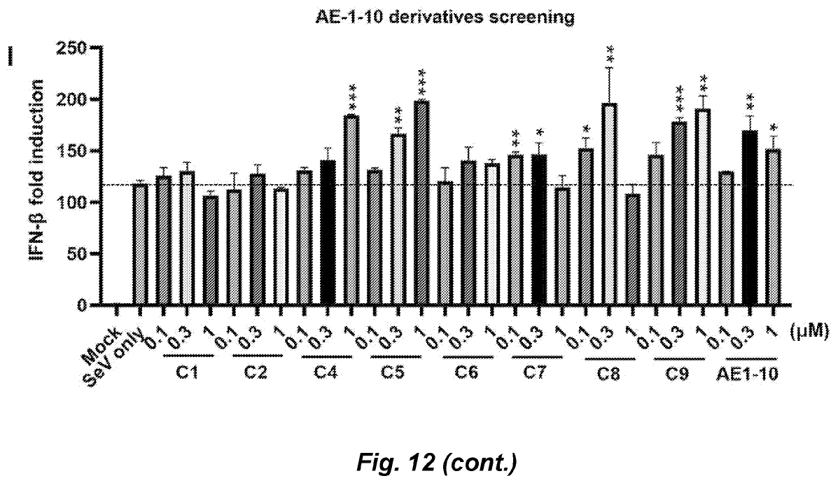

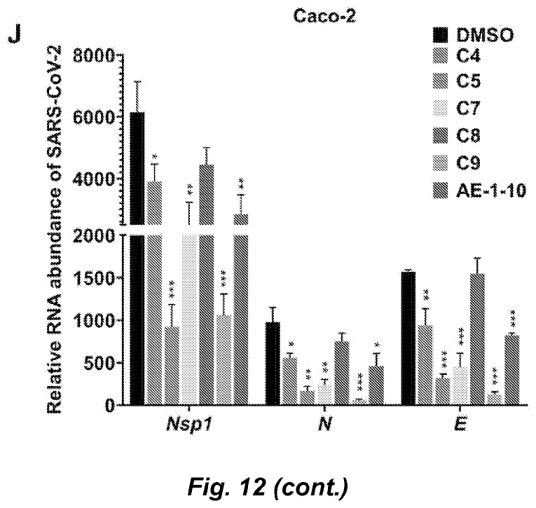

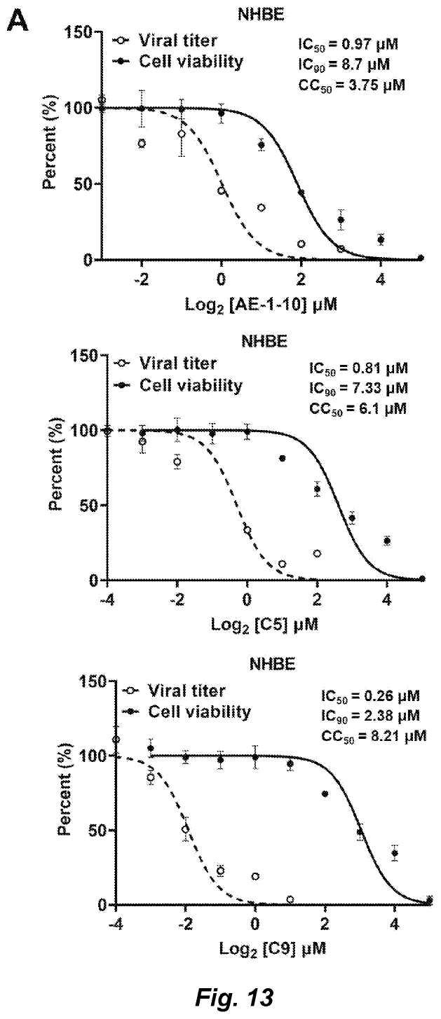

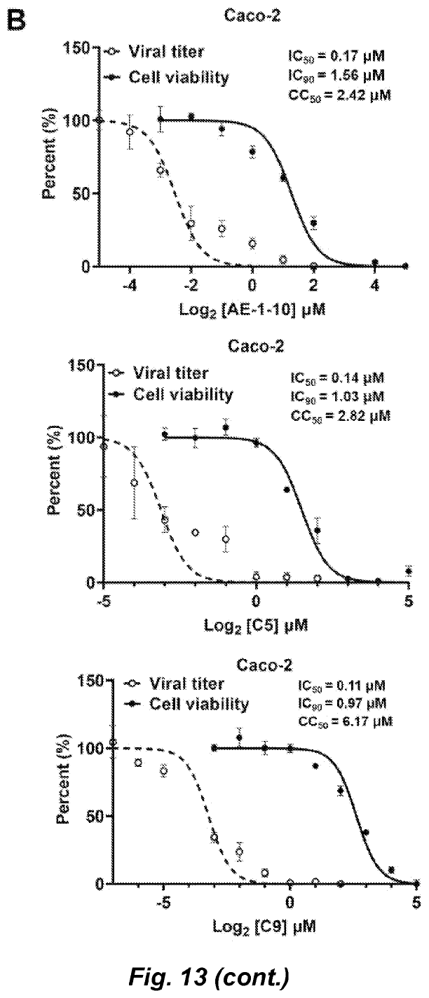

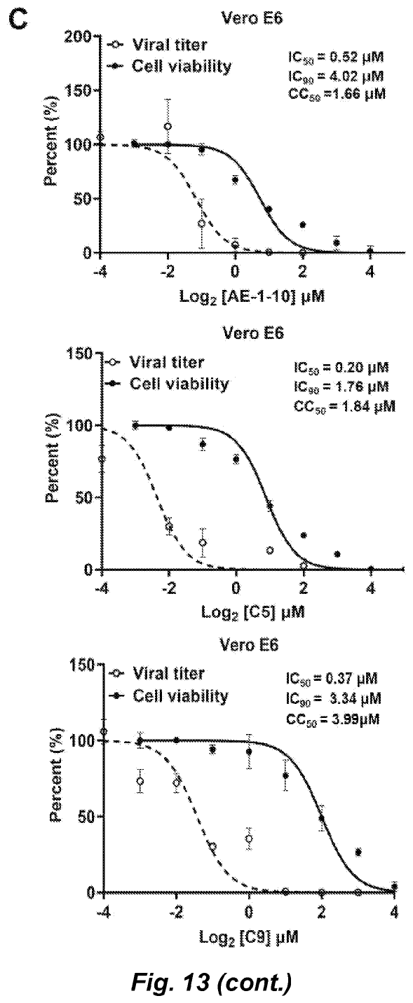

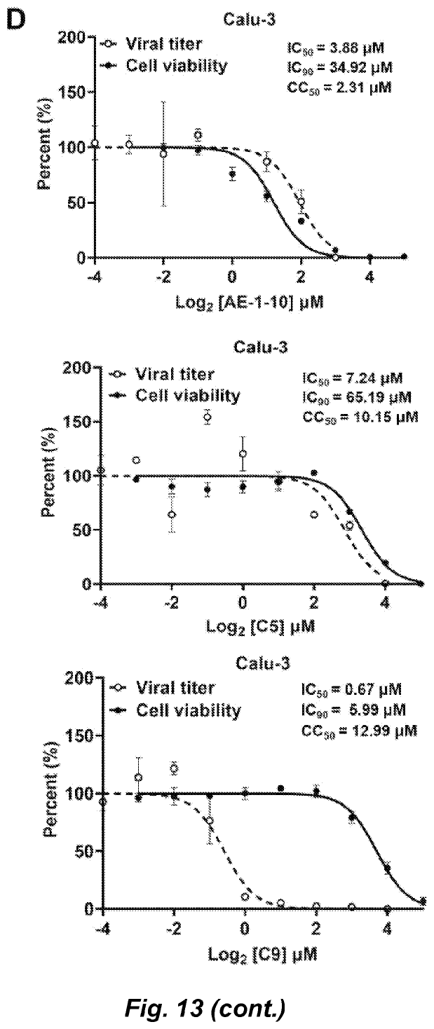

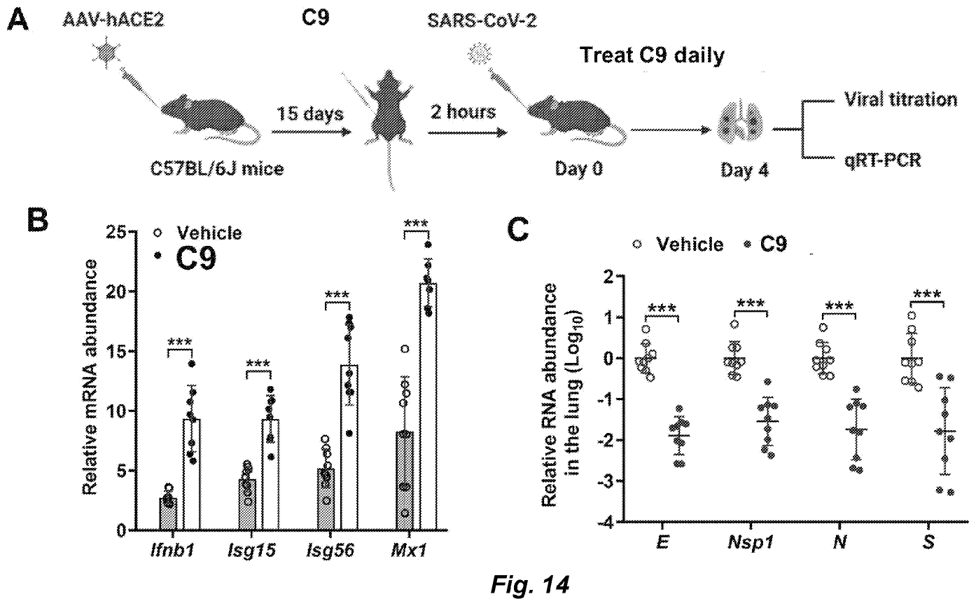

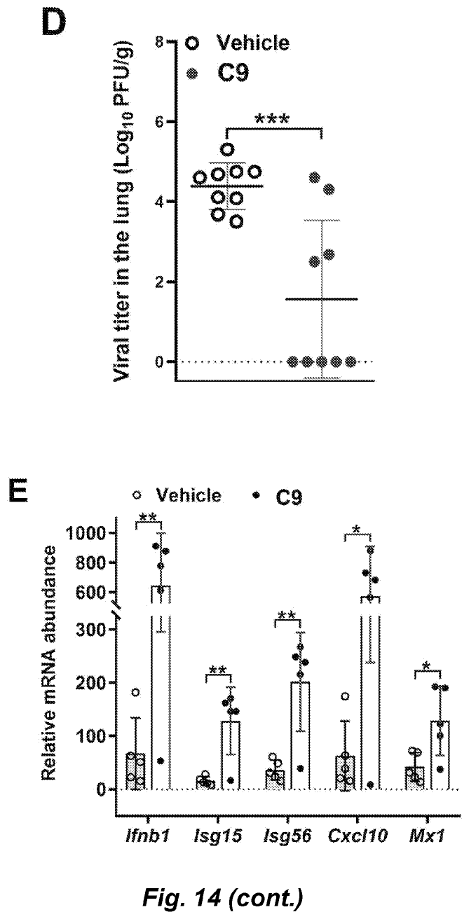

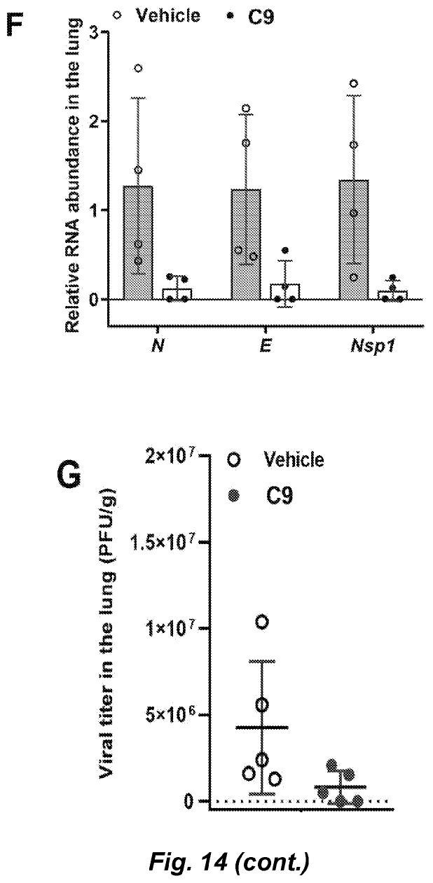

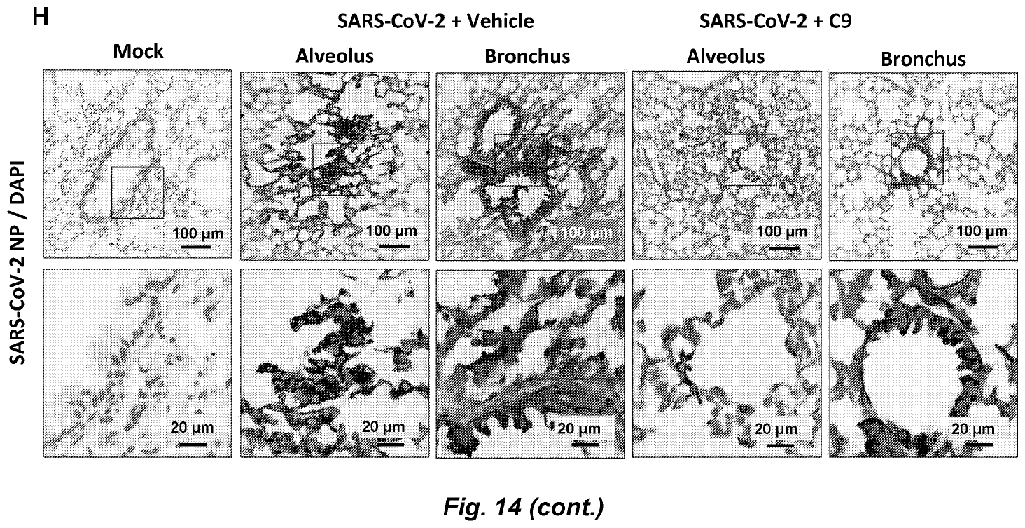

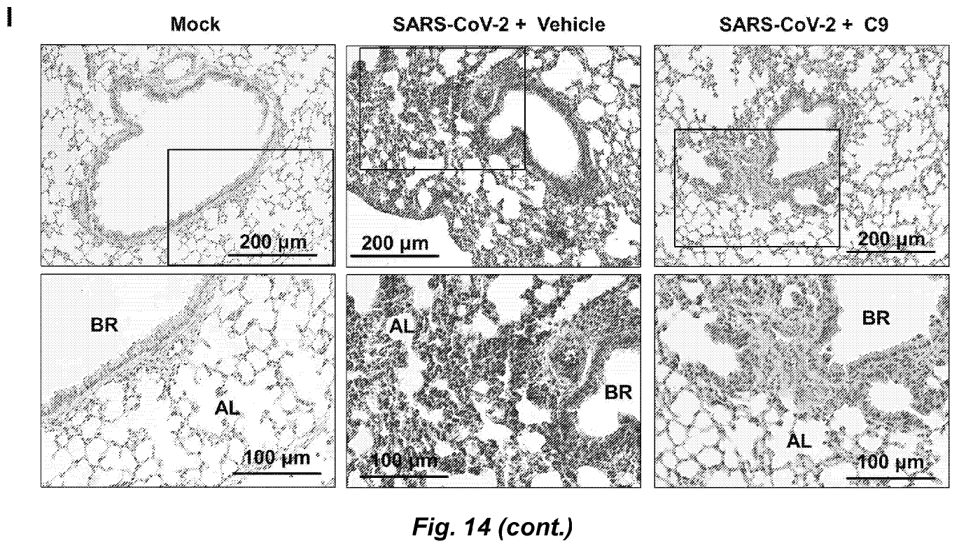

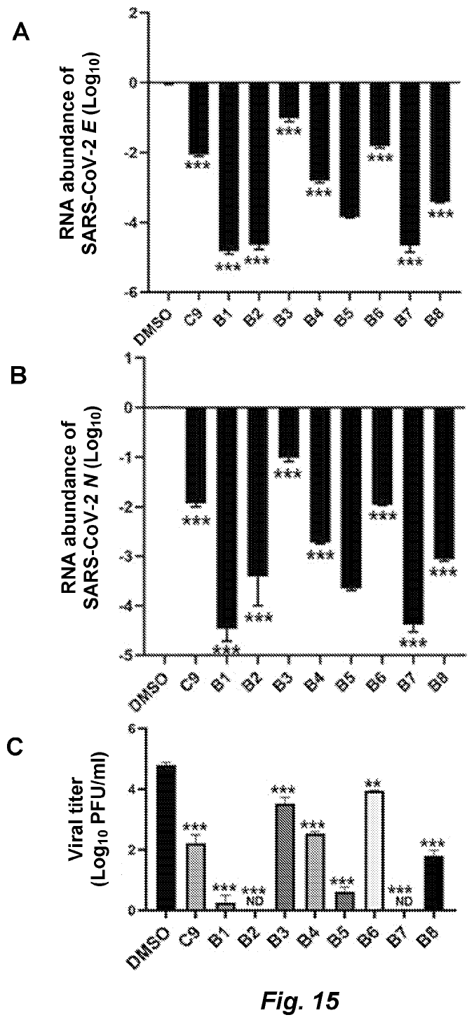

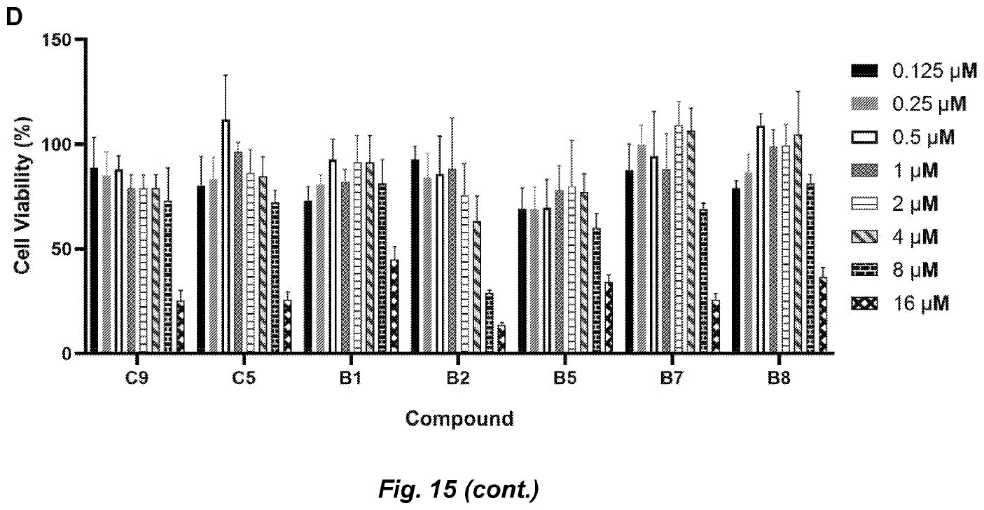

Identification of SARS-CoV-2 Proteins that Induce IRF3 Deamidation and Inhibit IFN Induction. SARS-CoV-2 is highly infectious and transmissible in human population. Ongoing research is keen on viral entry, how viral post-entry mechanisms contribute to the SARS-CoV-2 infection is not understood. We hypothesize that SARS-CoV-2 encodes a number of viral polypeptides to modulate host innate immune response, thereby promoting viral replication and dissemination. To test this idea, we first compared the antiviral gene expression induced by SARS-CoV-2, with that by Sendai virus, a prototype RNA virus. In normal human bronchial epithelial cells (NHBE), Sendai virus induced a rapid and robust expression of all antiviral genes with a peak at 6 hour post-infection (hpi) and an increase fold ranging from ˜15,000 (for ISG15) to 800,000 (for IFNB1) (FIG. 1A). By stark contrast, SARS-CoV-2 induced a weak and delayed expression of antiviral genes, peaking at 96 hpi (FIG. 1A). The fold induction of these antiviral genes by SARS-CoV-2 was roughly three orders of magnitude lower than that induced by Sendai virus. Similar patterns were observed in human Calu-3 lung cancer cells and Caco-2 colorectal cancer cells, two cell lines that support robust SARS-CoV-2 replication (FIGS. 7A and 7B). Interestingly, although delayed, the expression of Mx1 in Calu-3 and Caco-2 cells induced by SARS-CoV-2 was as robust as that induced by Sendai virus. To determine whether RNA derived from SARS-CoV-2-infected cells is able to trigger innate immune activation, we extracted total RNA from SARS-CoV-2-infected NHBE cells and, along with poly(I:C) transfected into NHBE cells. When mRNA of IFNB1, ISG15, ISG56, CCLS and Mx1 was analyzed, we found that total RNA extracted from SARS-CoV-2-infected NHBE cells, but not that from mock-infected NHBE cells, induced antiviral gene expression as potently as poly(I:C) (FIG. 1B). These results suggest that SARS-CoV-2 inhibits antiviral innate immune defense.

To understand the viral mechanisms of immune modulation, we screened viral polypeptides with an expression library for IFN-β induction by Sendai virus. This reporter assay identified several SARS-CoV-2 proteins, including ORF6, ORF7b, ORFS, ORF9b, N, Nsp7, Nsp8 and Nsp13, that inhibit IFN induction to various extent (FIG. 1C). To validate, we transfected 293T cells with plasmids expressing these individual viral polypeptides and found that all six viral polypeptides inhibited the expression of antiviral genes, including IFNB1, ISG56 and CCLS, in response to Sendai virus infection (FIG. 1D). Importantly, ORF6 and N were previously reported to inhibit the nuclear important of transcription factors (e.g., IRF3) and sequester viral double-stranded RNA, respectively, to suppress innate immune defense. Thus, we further examined the other six viral proteins for the inhibition of IFN induction. Nevertheless, these results demonstrate that multiple viral polypeptides can inhibit IFN induction.

SARS-CoV-2 is a coronavirus that likely induces innate immune activation via RNA sensors such as RIG-I and MDA5. To determine the target of inhibition, we over-expressed key components of this pathway, including RIG-I-N (2CARD-only), MAVS, TBK1 and IRF3, and examined IFN-β induction by reporter assay (FIG. 7C). Notably, the RIG-I-N is a constitutively active form of RIG-I independent of RNA ligand. This assay showed that ORF7b, ORF8 and Nsp13 can significantly inhibit IFN induction by the expression of more than one component of the RIG-I-IFN pathway, while ORF9b, Nsp7 and Nsp8 had no significant inhibition of IFN induction by ectopic expression (FIG. 7D). Further analysis demonstrated that ORF8 and Nsp13 potently inhibited IRF3-mediated IFN induction, suggesting that IRF3 is the point of inhibition (FIG. 1E). Thus, we focused on IRF3 regulation by these viral polypeptides, with an interest in deamidation, a process that can be catalyzed by metabolic glutamine amidotransferases. When endogenous IRF3 was analyzed by two-dimensional gel electrophoresis, we found that ORF7b, ORF8, NSP8 and NSP13 induced a shift of IRF3 toward the negative side of the gel strip, suggesting the deamidation of IRF3 (FIG. 1F). Interestingly, expression of N induced a shift of IRF3 in the upper-left direction, suggesting possible phosphorylation. We also investigated interaction between IRF3 and these viral proteins by co-immunoprecipitation (Co-IP). As shown in FIG. 1G, IRF3 directly interacted with ORF7b, ORF8, and ORF9b, but not ORF6, Nsp7, Nsp8 and Nsp13.These results collectively show that SARS-CoV-2 targets IRF3 for inhibition.

Identification of CTPS1 that Inhibits IFN Induction. Given that multiple SARS-CoV-2 viral polypeptides induce IRF3 deamidation, we reasoned that a cellular enzyme catalyzes IRF3 deamidation. The human genome encodes 11 glutamine amidotransferases that can potentially function as protein deamidases. With shRNA-mediated knockdown, we screened for cellular GATs whose knockdown increase IFN induction by Sendai virus infection. This experiment showed that depletion of CTPS1 elevated Sendai virus-induced IFN expression by ˜1.5-fold (FIG. 2A). Remarkably, depletion of the closely-related CTPS2 had no significant effect on IFN induction. The knockdown efficiency of these cellular GATs was validated in our recent publications. Notably, depletion of several GATs, including PPAT, ASNS and NADSYN1, reduced IFN induction, possibly due to the essential roles of these metabolic enzymes in cell proliferation and survival. To examine the role of CTPS1 in innate immune defense, we constructed 293T cell lines that express two independent shRNA and validated CTPS1 depletion by quantitative real-time PCR and immunoblotting (FIGS. 2B and 8A). Compared with control cells, Sendai virus infection induced higher transcript levels of IFNB1, CCLS, ISG56 and ISG15, in CTPS1-depleted 293T cells as analyzed by real-time PCR (FIG. 2C). The elevated production of IFN-β and CCL5 was further confirmed by ELISA (FIG. 2D). Similarly, depletion of CTPS1 in human THP-1 monocytes (FIG. 2E) elevated IFN-β and CCL5 expression and production in response to Sendai virus infection (FIGS. 2F and 8B). Consistent with the elevated antiviral immune response, depletion of CTPS1 reduced the replication of vesicular stomatitis virus (VSV) by —10-fold in 293T cells (FIG. 2G). These results collectively demonstrate that CTPS1 negatively regulates IFN induction in response to Sendai virus infection.

To assess the role of CTPS1 in SARS-CoV-2 infection, we depleted CTPS1 in NHBE cells (FIG. 2H) and assess antiviral gene expression and viral replication. Real-time PCR analysis indicated that depletion of CTPS1 increased the expression of IFNB1, ISG15, ISG56 and CCL5 by a factor of ranging from ˜15 (for ISG15 and 56), to 1000 (for CCL5) (FIG. 21). Conversely, depletion of CTPS1 reduced SARS-CoV-2 RNA abundance by two-fold for N and E genes, and >10-fold for Nsp1 gene (FIG. 2J), which correlated with four-fold reduction in viral titer in the medium at 48 h post-infection (FIG. 2K). Similar results were observed in Caco-2 cells for elevated antiviral gene expression in response to SARS-CoV-2 infection upon CTPS1 depletion (FIGS. 8C and 8D). This result also correlated with reduced SARS-CoV-2 replication as analyzed by real-time PCR for viral gene expression and plaque assay for infectious virions in the medium (FIGS. 8E and 8F). These results show that CTPS1 negatively regulates antiviral immune response against SARS-CoV-2 and deficiency in CTPS1 promotes antiviral gene expression to impede SARS-CoV-2 replication.

CTPS1 Interacts with and Deamidates IRF3. To determine the point of inhibition, we used the ectopic expression system as described in FIG. 7C. Reporter assay indicated that depletion of CTPS1 increased the IFN-β induction activated by the ectopic expression of all components, including RIG-I-N, TBK1 and IRF3-5D, of the RIG-I-IFN pathway (FIG. 3A). The specific effect of CTPS1 depletion on IFN-β expression induced by IRF3-5D supports the conclusion that deamidation likely targets IRF3 for inhibition (FIG. 9A). CTPS1 is a metabolic enzyme that catalyzes the synthesis of CTP from UTP and glutamine. We hypothesize that CTPS1 targets IRF3 for deamidation to inhibit IFN induction. To test this hypothesis, we first determined whether the enzyme activity of CTPS1 is required for its inhibition. We generated the enzyme-deficient mutant (C399A/H526A/E528A) of CTPS1 (CTPS1-ED) and performed a reporter assay. This result showed that wild-type CTPS1, but not the enzyme-deficient mutant, inhibited IFN-β induction induced by Sendai virus infection in a dose-dependent manner (FIG. 9B). Furthermore, an inhibitor of cellular GATs, 6-diazo-5-oxo-L-norleucine (DON) elevated Sendai virus-induced IFN-β gene expression in both CTPS1 depleted and control 293T cells (FIG. 9C). These results support the conclusion that the enzyme activity of CTPS1 is necessary for the inhibition of IFN induction.

Next, we assessed whether CTPS1 interacts with IRF3 by co-immunoprecipitation (Co-IP). As shown in FIG. 3B, CTPS1 was readily detected in protein complexes precipitated with antibody against IRF3, indicating the physical interaction between IRF3 and CTPS1. The interaction between IRF3 and CTPS1 was confirmed by co-IP assay from transiently transfected 293T cells (FIG. 9D). However, CTPS2 failed to interact with IRF3. To determine whether CTPS1 is a possible deamidase, we depleted CTPS1 with shRNA and examined IRF3 charge by two-dimensional gel electrophoresis. Indeed, knockdown of CTPS1 shifted IRF3 toward the negative charge end of the gel strip, indicating the increased charge due to CTPS1 depletion (FIG. 3C). Similarly, the ectopic expression of the enzyme-deficient CTPS1-ED mutant also shifted IRF3 toward the negative pole of the gel strip (FIG. 3D). We then sought to identify the site of deamidation using tandem mass spectrometry. We purified IRF3 in transfected 293T cells, without or with the ectopic expression of CTPS1-ED. Tandem mass spectrometry analysis consistently identified N85, N389 and N397 as deamidation sites Deamidation of Q15, N184 and N217 was observed to much less extent. We generated IRF3 mutants containing individual deamidated residues, i.e., Q>E and N>D mutations, and analyzed their activity in IFN induction. Remarkably, the N85D mutation nearly deprived IRF3 to activate the IFNB1 promoter, while N389D and N397D elevated the activity of IRF3 to do so (FIGS. 3E and 9E). Given that the phenotype of the IRF3-N85D mutant is consistent with the inhibition of CTPS1-mediated deamidation, we focused on this mutant in this study.

To test whether CTPS1 is a deamidase of IRF3, we purified GST-IRF3, CTPS1 wild-type and the enzyme-deficient CTPS1-ED mutant from 293T cells to high homogeneity (FIG. 3F), and performed IRF3 in vitro deamidation assay. When IRF3 was analyzed by two-dimensional gel electrophoresis, CTPS1, but not the CTPS1-ED mutant, shifted IRF3 toward the positive end of the gel strip, indicative of deamidation (FIG. 3G). This result indicates that CTPS1 can function as a bona fide deamidase of IRF3. To assess the specificity of CTPS1-mediated deamidation, we used the deamidated IRF3-N85D mutant and CTPS1 depletion for IRF3 charge analysis. Two-dimensional gel electrophoresis analysis indicated that depletion of CTPS1 shifted IRF3 wild-type toward the negative pole of the gel strip, while had no effect on the deamidated IRF3-N85D (FIG. 3H). Similarly, depletion of CTPS1 failed to shift IRF3-N85A and IRF3-N85Q, indicating that IRF3-N85A and IRF3-N85Q are resistant to CTPS1-mediated deamidation (FIG. 9F). Consistent with this result, IRF3-N85A demonstrated higher IFN induction by reporter assay compared with wild-type IRF3 (FIG. 9G). However, IRF3-N85Q had lower IFN induction compared to wild-type, likely due to steric hindrance in DNA-binding (see next section). Together, these results support the conclusion that CTPS1 targets N85 of IRF3 for deamidation.

Deamidated IRF3 Fails to Bind Cognate Responsive Elements within Promoters of

Pro-inflammatory Genes. CTPS1 negatively impacts IFN induction, we thus probed the deamidation in regulating IRF3-mediated inflammatory gene expression using the deamidated IRF3-N85D and deamidation-resistant IRF3-N85A mutants. First, we “reconstituted” IRF3 expression in Irf3^{-/-} Irf7^{-/-} mouse embryonic fibroblasts (MEFs) (FIG. 4A) and examined antiviral immune response upon Sendai virus infection. Real-time PCR analysis indicated that IRF3-N85D, compared with IRF3 wild-type, failed to induce the expression of IFNs and IFN-stimulated genes (FIG. 4B). By contrast, the deamidation-resistant IRF3-N85A mutant more potently activated the expression of these IFNs and ISGs than wild-type IRF3 (FIG. 4C). To profile the global gene expression in MEFs “reconstituted” with IRF3-N85D, we performed

RNA sequencing and discovered that “reconstituted” expression of wild-type IRF3 activated the expression of a broad spectrum of IFNs and ISGs, while that of IRF3-N85D failed to do so. The ability of IRF3 wild-type, IRF3-N85D and IRF3-N85A to activate antiviral gene expression also correlated with IFN-β and CXCL10 production in the medium in response to Sendai virus infection (FIGS. 4D and 4E). These results show that deamidation inhibits IRF3-mediated expression of IFNs and ISGs.

In response to Sendai virus infection, IRF3 undergoes phosphorylation, dimerization and nuclear translocation to activate the expression of inflammatory genes. To probe the effect of deamidation on IRF3 activation, we analyzed these events of IRF3 activation using wild-type IRF3 and IRF3-N85D. Immunoblotting analyses, with phosphor-specific antibody for IRF3 and native gel electrophoresis, indicated that IRF3-N85D was phosphorylated and dimerized at higher levels than wild-type IRF3 (FIG. 10A). Immunofluorescence microscopy analysis showed that wild-type IRF3 and IRF3-N85D accumulated in the nucleus at similar rate (FIGS. 10B and 10C). Thus, deamidation did not impair the phosphorylation, dimerization and nuclear translocation of IRF3 when activated by Sendai virus infection.

In collaboration with other transcription factors, IRF3 acts in concert in the so-called “enhancesome” of the IFN-β promoter, which has been well characterized by structural studies. In the structure of the DNA-protein complex, the N85 residue of IRF3 makes a direct contact with the backbone of dsDNA via hydrogen bond. Deamidation of N85 is predicted to disrupt the hydrogen bond and create a negative charge that will repel the highly negatively charged backbone of dsDNA. Thus, we performed chromosome immunoprecipitation and quantified DNA by real-time PCR. Wild-type IRF3, but not IRF3-N85D, enriched the sequences of IFNs, including Ifnb and Ifna4, in response to Sendai virus infection (FIG. 4F). Consistent with this, an in vitro gel shift assay using purified IRF3 proteins indicated that wild-type IRF3 bound to its cognate consensus sequence and IRF3-N85D failed to do so (FIG. 4G). These results collectively show that deamidation impairs IRF3 to bind to its responsive element in promoters of inflammatory genes.

To determine the effect of IRF3 deamidation on SARS-CoV-2 replication, we first infected MEFs “reconstituted” with wild-type IRF3 and IRF3-N85D with GFP-marked VSV. We found that wild-type IRF3 markedly reduced VSV replication by immunofluorescence microscopy and plaque assay (FIGS. 10D and 10E). By contrast, IRF3-N85D only marginally reduced VSV replication. Next, we examined SARS-CoV-2 replication in these cells. To facilitate SARS-CoV-2 infection, we established MEF cell lines stably expressing human ACE2 (FIG. 10F). These “reconstituted” MEFs were infected with SARS-CoV-2 and examined for innate immune response. Quantitative real-time analyses indicated that wild-type IRF3 induced modest level of expression of Ifnb, Isg15, Isg56 and Cxc110 (FIG. 10G). While IRF3-N85A more robustly induced gene expression, IRF3-N85D had minimal induction of these genes. Conversely, wild-type IRF3 and IRF3-N85A reduced viral RNAs by ˜40% to 70%, while IRF3-N85D had no apparent effect on the RNA levels of Nsp1 and E, or reduced N RNA by ˜45%. Plaque assay further showed that wild-type IRF3 and IRF3-N85A diminished infectious SARS-CoV in the medium by 75% and 90%, respectively, while IRF3-N85D only reduced by ˜30% (FIG. 4H). Thus, deamidation impairs IRF3 to defeat SARS-CoV-2 replication.

SARS-CoV-2 Nsp8 and ORF8 Induce IRF3 Deamidation via CTPS1. To probe the virus-host interaction underpinning CTPS1-mediated IRF3 deamidation, we then examined viral proteins that interact with CTPS1 using the SARS-CoV-2 expression library. A co-IP assay identified multiple SARS-CoV-2 polypeptides that co-precipitated with CTPS1 in transfected 293T cells, including ORF7b, ORF8, M, Nsp8, Nsp10 and Nsp14 (FIG. 11A). The interaction between CTPS1 and these SARS-CoV-2 proteins were further validated by co-IP assay using endogenous CTPS1 (FIG. 5A). Three out of the six CTPS1-interacting SARS-CoV-2 polypeptides, i.e., ORF7b, ORF8 and Nsp8, also induced IRF3 deamidation in transfected 293T cells (FIG. 1D). We reasoned that these SARS-CoV-2 polypeptides usurp CTPS1 to promote IRF3 deamidation. To test this, we depleted CTPS1 with shRNA and examined IRF3 deamidation. Indeed, depletion of CTPS1 shifted IRF3 toward the negative side of the gel strip (FIG. 5B). Apparent shift of IRF3 was observed in the presence of ORF8 and NSP8, a minor portion of IRF3 was shifted with ORF7b expression, when CTPS1 was depleted (FIG. 5B). To further validate this, we used wild-type IRF3 and the deamidation-resistant IRF3-N85A for two-dimension gel electrophoresis. This revealed that wild-type IRF3, but not the IRF3-N85A, was shifted by ORF8 and Nsp8 expression (FIG. 5C). Interestingly, ORF7b expression shifted both wild-type IRF3 and IRF3-N85A, suggesting that ORF7b induces the deamidation of IRF3 at sites other than N85. Nevertheless, these results show that ORF8 and NSP8 induce the CTPS1-mediated deamidation of N85 of IRF3.

To dissect the mechanism by which SARS-CoV-2 polypeptides promote CTPS1-mediated deamidation of IRF3, we determined whether ORF8 and Nsp8 impact the CTPS1-IRF3 interaction by co-IP assays. In 293T cells transiently expressing ORF8, more CTPS1 was precipitated by IRF3, indicating an elevated interaction between CTPS1 and IRF3 (FIG. 5D). However, Nsp8 had no effect on this interaction. Next, we determined whether ORF8 and Nsp8 affect CTPS1 activity to deamidate IRF3. To do that, we purified CTPS1, without or with SARS-CoV-2 polypeptides, from transfected 293T cells and performed in vitro deamidation assay. This analysis indicated that ORF8, and to a lesser extent, Nsp8 increased CTPS1 activity to deamidate IRF3 (FIG. 5E). Together, these results collectively show that SARS-CoV-2 polypeptides can promote CTPS1 to deamidate IRF3.

SARS-CoV-2 ORF8 Activates CTPS1 to Promote Nucleotide Synthesis. CTPS1 is responsible for the synthesis of CTP that is crucial for a balanced nucleotide pool during cell proliferation and viral replication. Activated nucleotide synthesis is likely to favor transcription and genome replication during SARS-CoV-2 infection. We then examined the metabolite of the glycolysis and nucleotide synthesis pathways. In colorectal cancer Caco-2 cell line that supports SARS-CoV-2 replication, we found that SARS-CoV-2 infection had no significant effect on the intracellular concentration of CTP. However, the relative concentration of UTP and UDP, and to a less extent UMP, immediate precursors of CTP, was significantly increased in Caco-2 cells at 72 h after SARS-CoV-2 infection. Strikingly, CTP and UTP were significantly decreased at 96 h post-infection. These results support the rate-limiting role of CTP synthetases in catalyzing UTP to CTP conversion and the decrease of CTP and UTP at 96 hpi is likely due to a rapid consumption. To determine the rate of synthesis that reflects the activity of CTPS1, we analyzed CTP synthesis using isotope tracing with [ 15 N]glutamine (FIG. 5E). Compared with mock-infected cells, SARS-CoV-2 increased the labeled (M+1) CTP by >2-fold in Caco-2 cells with a 30-minute tracing (FIG. 5F). Interestingly, SARS-CoV-2 infection had no apparent effect on the [¹⁵N]UTP (M+1) under similar conditions, indicating the specificity of CTPS1 activated during SARS-CoV-2 infection. The [¹⁵N]UTP (M+1) is the product of the de novo pyrimidine synthesis where CAD catalyzes dihydroorotate synthesis using glutamine. Next, we established Caco-2 cell lines that stably express SARS-CoV-2 polypeptides, including ORF7b, ORF8 and Nsp8. When flux analysis with [¹⁵ N]glutamine was performed, we found that cells expressing ORF7b and ORF8 had >3- and 5-fold more [¹⁵N]CTP (M+1) compared to control cells (Vector group), respectively (FIG. 5G). Consistently, ORF7b and ORF8 also increased [¹⁵N]CDP (M+1), presumably a product hydrolyzed from CTP. However, Nsp8 expression had no apparent effect on labeled [¹⁵N]CTP. Similar results were also observed for these SARS-CoV-2 polypeptides in LoVo colorectal cancer cells that support SARS-CoV-2 replication (FIG. 11B and FIG. 11C). These results show that ORF7b and ORF8 promote CTP synthesis.

To probe the effect of ORF7b and ORF8 on the enzyme activity of CTPS1, we purified CTPS1 from stable 293T cells with transient expression of ORF7b and ORF8, and performed in vitro CTP synthesis reaction to characterize the kinetic parameters K_{cat} and Kₘ of CTPS1 (FIG. 5H). As showed in FIG. 51 and Table 1, Both ORF7b and ORF8 elevated the K_{cat} of CTPS1, and ORF8 also increase Kₘ of CTPS1.

TABLE 1
Kinetic constants for CTP synthetase.

| | Kₘ (mM) | K_{cat} (min⁻¹) |
|---|---|---|
| Vector | 30.94 ± 10.45 | 0.44 ± 0.09 |
| ORF7b | 30.54 ± 21.53 | 0.88 ± 0.36 |
| ORF8 | 55.20 ± 50.29 | 1.27 ± 0.82 |

Kₘ and K_{cat} were calculated by Michaelis-Menten equation.

Inhibitors of CTPS1 Impede SARS-CoV-2 Replication. Inhibition of CTPS1 is expected to reduce CTP supply and restore IFN induction, thereby impeding SARS-CoV-2 replication. We sought to develop small molecule inhibitors of CTPS1 to defeat SARS-CoV-2 infection and COVID-19. In an experiment to identify cellular targets of Q6ca that demonstrates antiviral activity, we performed click-based cross-linking, affinity purification and mass spectrometry analysis. This led to the identification of CTPS1 as a cellular target of Q6ca, although ranked lower than several other proteins (data not shown). With Q6ca as the lead scaffold, we derivatized three molecules and tested whether they inhibited IRF3 deamidation. As analyzed by two-dimensional gel electrophoresis, we found that AE-1-10 demonstrated the best efficacy to inhibit IRF3 deamidation in Caco-2 cells expressing ORF8 or A549 cells expressing Nsp8, although the other three molecules had modest inhibition as well (FIGS. 6A and 12A). Accordingly, AE-1-10 increased IFNB1 expression in a dose-dependent manner in 293T cells infected with Sendai virus, while the other three molecules had marginal effect (FIG. 12B). To determine the specificity of AE-1-10, we depleted CTPS1 in 293T cells for luciferase report assay. We found that AE-1-10 elevated IFN induction in control 293T cells but failed to do so in CTPS1-depleted 293T cells and had no effect on NF-κB activation (FIGS. 6B and 12C). To validate that AE-1-10 targets CTPS1, we employed BE-2-10, a close relative of AE-1-10 with a reactive alkyne warhead, for biochemical labeling using 293T cells expressing CTPS1. This assay showed that BE-1-10 reacted with CTPS1 in a dose-dependent manner (FIG. 6C). Taken together, these results demonstrate that AE-1-10 inhibits CTPS1 to elevate IFN induction.

Activated CTPS1 also increases CTP supply in cells infected with SARS-CoV-2 to facilitate viral replication. With Caco-2 cells that stably express ORF8, we performed [¹⁵N]glutamine flux analysis with AE-1-10 treatment. As shown in FIG. 6D, AE-1-10 treatment reduced [¹⁵N]CTP and, much more so [¹⁵N]CDP in a dose-dependent manner. Similar reduction was observed in ORF8 expressed LoVo cells after AE-1-10 treatment (FIG. 12D). Similarly, AE-1-10 also reduced the intracellular concentration of [¹⁵N]CTP and [¹⁵N]CDP in Caco-2 cells infected with SARS-CoV-2 (FIG. 6E). These results show that an inhibitor of CTPS1 can reduce CTP synthesis in SARS-CoV-2-infected cells and in cells expressing ORF8.

To probe the biological significance of AE-1-10 treatment, we analyzed the expression of antiviral genes, including IFNB1, ISG56, CCLS and Mx1, in SARS-CoV-2-infected Caco-2 cells. Real-time PCR analysis indicated that AE-1-10 elevated the expression of these antiviral genes at the concentration of 2 and 6 μM (FIG. 6F). Consistent with the elevated antiviral gene expression, the expression of viral genes, including Nsp1, N and E, was reduced by AE-1-10 in a dose-dependent manner (FIG. 6G), with more than 50% and 75% reduction at the concentration of 2 and 6 μM, respectively. The reduced viral gene expression also correlated with lower viral yield, in which AE-1-10 reduced viral yield by —10- and 100-fold at the concentrations of 2 and 6 μM (FIG. 6H). Similar results were observed in SARS-CoV-2-infected NHBE cells when treated with AE-1-10, including elevated antiviral gene expression and reduced viral RNA and yield (FIG. 12E-12G). These results collectively show that AE-1-10 inhibits CTPS1 to impede nucleotide synthesis and restore IFN induction, thus synergizing to diminish SARS-CoV-2 replication.

To improve the antiviral potency of AE-1-10, we designed nine more molecules based on AE-1-10, named them C1-C9, and successfully obtained eight out of 9, except C3. NMR and mass spectrometry analyses indicate that these molecules were homogenous and demonstrated physical-chemical properties consistent with their molecular scaffolds (data not shown). Cell toxicity test showed that these molecules had no significant effect on cell viability up to 3 μM and reduced cell viability and proliferation at 9 μM, likely due to the diminished CTP supply when CTPS1 was inhibited (FIG. 12H). An IFN induction reporter assay showed that four out of eight derivatives, including C4, C5, C8 and C9, had improved effect to increase IFN induction, compared with AE-1-10 (FIG. 121). Whereas C6 and C7 demonstrated modest effect, C1 and C2 had no effect on IFN induction. Thus, we selected five derivatives, including C4, C5, C7, C8 and C9 for further SARS-CoV-2 study. Consistent with their ability to enhance IFN induction, C9 and C5 showed more robust antiviral activity in SARS-CoV-2 infection as determined by plaque assay (FIG. 6I), compared to AE-1-10. The result of plaque assay also correlated with SARS-CoV-2 gene expression in Caco-2 cells (FIG. 12J). These results identified a number of CTPS1 inhibitors that potently antagonize SARS-CoV-2 replication in cultured cells. Further antiviral activity and cytotoxicity in several cell lines are shown in FIG. 13A-D. And the C9 compound was shown to protect against infection with SARS-CoV-2 infection (FIG. 14A-G).

Built on data from the C series of small molecules, we further synthesized a new B series compounds and tested their antiviral activity against SARS-CoV-2. When viral RNA was examined by real-time PCR analysis, we found that treatment of B1, B2 and B7 reduced viral RNA abundance by the order of 5 magnitude compared to the vehicle (DMSO) (FIG. 15A and FIG. 15B). Consistent with previous data, C9 reduced viral RNA abundance by an order of magnitude of 2. The other three compounds, B4, B5 and B8 also had significant improvement compared to C9. These results also correlated with viral titer as determined by plaque assay. Specifically, compounds B1, B2, B7 reduced viral titer to the limit of detection, while B5 and B8 had better effect than C9 that reduced SARS-CoV-2 titer by an order of 3 magnitude (FIG. 15C). Importantly, when these compounds were used to treat Caco-2 cells without SARS-CoV-2, cell viability and proliferation were not significantly affected up to 4μM, indicating that the antiviral activity of these small molecules is not derived from their cytotoxicity (FIG. 15D).

Studies involving cultured cells, model animals and COVID-19 patients indicate that SARS-CoV-2 effectively inhibits the production of type I and III interferons. However, the molecular mechanism by which SARS-CoV-2 does so is not understood. Earlier works comparing SARS-CoV-2 genome sequences to those of other beta coronaviruses, particularly SARS-CoV and MERS-CoV, predict putative viral polypeptides in modulating host innate immune defense, including IFN induction. Here, we report that SARS-CoV-2 deploys multiple proteins to activate CTPS1, which promotes CTP synthesis, while inactivates IRF3 and mutes IFN induction. Remarkably, pharmacological inhibition of CTPS1 potently impedes CTP synthesis and effectively restores IFN induction, thereby diminishing SARS-CoV-2 replication and offering an antiviral strategy targeting a host enzyme.

Dysregulated immune response, described as “cytokine storm”, is a characteristic shared among COVID-19 patients under severe and critical. Among the skewed cytokine profile, type I IFNs are produced at very low or under detection levels, which likely contributes to the rapid replication of SARS-CoV-2 in these patients. However, recent reports indicate that the majority of severe and critical COVID-19 patients show low inflammatory cytokines, compared with patients infected with influenza virus. To dissect the mechanism of innate immune evasion by SARS-CoV-2, we first showed that RNA produced from SARS-CoV-2-infected NHBE cells is potent to induce IFN, whereas SARS-CoV-2 failed to do so during infection when compared with Sendai virus, suggesting that viral proteins antagonize IFN induction. Indeed, a screen utilizing the SARS-CoV-2 expression library identified ORF7b, ORF8, Nsp8 and Nsp13 as inhibitors of IFN induction. Further analysis showed that these SARS-CoV-2 polypeptides target IRF3 for post-translational modification. Given the ubiquitous role of type I IFN in host innate immune defense against viral infection, regulatory mechanisms governing IRF3 activation are expected to operate independent of cell type and tissue origin (Ivashkiv and Donlin, 2014; Stetson and Medzhitov, 2006). Upstream components, such as pattern recognition receptors and their cognate adaptors, may be tissue- and cell type-specific.

Accordingly, viral factors targeting these downstream components likely function in a tissue-dependent manner, and those meddling the downstream components, such as IRF3, are anticipated to work independent of tissues or organs. In the lung, the epithelial cells and pneumocytes of the airway and respiratory track are the first responders in IFN production during SARS-CoV-2 infection.

The SARS-CoV-2 polypeptides that induce IRF3 deamidation are relatively small and unlikely to function as intrinsic deamidases. Indeed, a focused shRNA-mediated screen targeting cellular glutamine amidotransferases (GATs) identified CTPS1 as a negative regulator of IFN induction. CTPS1 belongs to the cellular GAT family that is known for their metabolic functions in biosynthesis of cellular building blocks in preparation for proliferation. CTPS1 demonstrates intrinsic activity to deamidate IRF3 in vitro and in cells, which is dependent on the active site required for the glutamine-hydrolysis activity (known as glutaminase) in catalyzing CTP synthesis. This study adds CTPS1 to the growing list of protein deamidases that are originally known as cellular GATs, expanding the functional repertoire of protein deamidation and GATs in immune regulation. Interestingly, CTPS1 and CTPS2 share 74% amino acid homology and were predicted to be functionally redundant. CTPS1, but not CTPS2, interacts with IRF3 in cells, suggesting that these two closely-related enzymes are functionally distinct. Indeed, loss or deficiency of CTPS1 due to mutations was found to impair CTP synthesis in T cell proliferation and result in primary immune deficiency, despite that CTPS2 is highly expressed in T cells. Given the pivotal roles of CTPS1 in T cell-mediated adaptive immunity, it remains interesting whether the protein-deamidating activity of CTPS1 is important for T cell immune function.

Deamidation results in the loss of DNA-binding activity of IRF3 to its cognate sequences, supporting the role of deamidation in diminishing IFN induction by IRF3. This constitutes a strategy that viruses effectively shut down antiviral gene expression during infection. Similarly, cells may deploy this mechanism to curtail gene expression that is not essential during proliferation when CTPS1 is highly active. Such a mechanism is analogous to the CAD-mediated RelA deamidation that shunts RelA to transactivate the expression of key glycolytic enzymes in promoting carbon metabolism during S phase. SARS-CoV-2 hijacks CTPS1-mediated IRF3 deamidation to evade IFN induction during infection, which may explain previous observations that SARS-CoV-2 fails to induce IFN production in COVID-19 patients and in animal models. Intriguingly, deamidated IRF3, similar to deamidated RelA, translocates into the nucleus, suggesting that deamidated IRF3 may have unidentified functions relevant to biological processes in the nucleus.

Nucleotide supply is a rate-limiting factor for cell proliferation and virus replication. As intracellular obligate pathogens, viruses rely on cellular machinery for their macromolecular biosynthesis. During viral productive infection, nucleotides are used for transcription, translation (ribosome regeneration), genome replication and lipid synthesis for assembly and maturation. Not surprisingly, viruses often activate metabolic enzymes to fuel nucleotide synthesis in support of their replication (Sanchez and Lagunoff, 2015). We discovered that SARS-CoV-2 infection and the expression of ORF7b and ORF8 activate CTPS1 to promote de novo CTP synthesis, thereby fueling viral replication. Strikingly, activated CTPS1 also inhibits type I IFN induction via deamidating IRF3. Thus, SARS-CoV-2 couples the inhibition of type I IFN induction to CTP synthesis via activating CTPS1. This predicts that SARS-CoV-2 relies on CTPS1 for its replication, and conversely inhibiting CTPS1 likely impedes SARS-CoV-2 replication. Indeed, depletion and pharmacological inhibition of CTPS1 greatly diminished CTP synthesis and effectively restored antiviral IFN induction.

Here we report the development of several small molecules that target CTPS1 for inhibition. These molecules demonstrate activity to stimulate IFN induction, but not NF-□B activation. CTPS1 and CAD negatively regulate the IFN and NF-κB induction, respectively. The specific stimulation of IFN induction by AE-1-10 and its derivative thus suggests their inhibition on CTPS1, but not CAD. Furthermore, the effect of AE-1-10 on IFN induction was observed in wild-type cells and this effect was abolished in CTPS1-depleted cells, supporting the conclusion that AE-1-10 inhibits CTPS1 to boost IFN production. Indeed, a derivative of AE-1-10 carrying reactive warhead cross-links with CTPS1 by in vitro assay. These results collectively show that AE-1-10 targets CTPS1 to promote IFN induction. Given that CTPS1 is essential for cell proliferation, AE-1-10 potentially induces toxicity in proliferating cells. The premise is that SARS-CoV-2 activates CTPS1 to facilitate its replication, which permits the selective inhibition of CTPS1 with low dose of AE-1-10 and its derivatives. Additionally, we cannot exclude the possibility that AE-1-10 and its derivatives target cellular proteins other than CTPS1. Future experiments will be necessary to further optimize the conditions that minimize the side effect of these CTPS1 inhibitors.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. Nos. 4,992,478, 4,820,508, 4,608,392, and 4,559,157. Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Materials and Methods

Cell culture. HEK293T, A549, LoVo, IRF3/7^{-/-} mouse embryonic fibroblasts (MEFs) were cultured in Dulbecco' s modified Eagle's medium (DMEM, Hyclone). THP1 cells were cultured in RPMI 1640 medium. Caco-2, Calu-3 were cultured in MEM medium. All these media were supplemented with 10% fetal bovine serum (FBS, HyClone), penicillin (100 U/mL) and streptomycin (100 μg/mL) and maintained at 37 ° C. in a humidified atmosphere of 5% CO₂. Primary normal, human bronchial/tracheal epithelial cells (NHBE) were cultured in airway epithelial cell medium according to the ATCC recommendation.

Viruses. Sendai virus (SeV) was purchased from Charles River. Vesicular stomatitis virus (VSV) was amplified using Vero cells. SARS-CoV-2 was propagated in Vero E6 cells. All SARS-CoV-2 related viral propagation, viral infection, and vital titration were performed in biosafety level 3 (BSL-3) facility (USC).

SARS-CoV-2 propagation: Vero E6 cells were seeded at 1.5×10⁶ cells per T25 flask for 12 h. Cells were washed with FBS-free DMEM medium once, and infected with SARS-CoV-2 at MOI 0.005 in FBS-free DMEM medium. Cells were checked daily for cytopathic effect (CPE). Virus-containing medium was harvested when virus-induced CPE reached approximately 80% (around 72 h after viral infection), centrifuge at 3000 rpm for 5 min, and store at −80° C.

SARS-CoV-2 infection: NHBE (1.5×10⁵ cells), Calu-3 (5×10⁵ cells) or Caco-2 cells (2×10⁵ cells) were seeded in one well of 12-well plates. Cells were washed with FBS-free medium before viral infection. SARS-CoV-2 was diluted in 250 μl (per well) medium corresponding to the cell line. Viral infection was incubated on a rocker for 45 min at 37° C. Cells were washed with fresh medium, and medium containing 10% FBS was added.

SARS-CoV-2 viral titration (plaque assay): Vero E6 cells were seeded in 6- or 12-well plates. When cell confluence reaches 100%, cells were washed with FBS-free medium, and infected with serially diluted SARS-CoV-2. After infection, medium was removed, and overlay medium containing FBS-free 1×DMEM and 1% low-melting point agarose was added. At 72 h post infection, cells were fixed with 4% paraformaldehyde (PFA) overnight, and stained with 0.2% crystal violet. Plaques were counted on a light box.

Plasmids. Luciferase reporter plasmids for IFN-β, NF-κB promoters, RIG-I-N, MAVS, TBK1, IRF3-5D and shRNA for human glutamine amidotransferases (CTPS1, CTPS2, GFPT1, GFPT2, GMPS, PFAS, PPAT, CPS1, ASNS and NADSYN1) were described previously (Zhao et al. (2016b), Cell Host Microbe 20, 770-784, and Zhou et al. (2020), Nature 579, 270-273. A cDNA construct was used to amplify and clone CTPS1 into mammalian expression vectors. Point mutants of IRF3 and CTPS1, including IRF3-Q15E, IRF3-N85D, IRF3-N184D, IRF3-N217D, IRF3-N389D, IRF3-N397D, IRF3-N85A, IRF3-N85Q, and CTPS1 enzyme-deficient (CTPS1-ED) mutant (C399A/H526A/E528A) were generated by site-directed mutagenesis and confirmed by sequencing. Lentiviral expression constructs containing IRF3, CTPS1 and hACE2 were generated from pCDH-CMV-EF1-Puro or pCDH-CMV-EF1-Hygro by molecular cloning. pLVX-EFlalpha-2XStrep-IRES-Puro containing SARS-CoV-2 viral genes.

Quantitative Real-time PCR (qRT-PCR). Total RNA was extracted from mock- or virus-infected cells using TRIzol reagent (Invitrogen). cDNA was synthesized from one microgram total RNA using reverse transcriptase (Invitrogen) according to the manufacturer's instruction. Quantitative real-time PCR (qRT-PCR) reaction was performed with SYBR Green Master Mix (Sigma) or qPCRBIO SyGreen Blue Mix Lo-ROX (Genesee Scientific). Relative mRNA abundance was calculated by 2-AAct method. Primers for qRT-PCR were listed in Table 3.

Lentivirus-mediated Stable Cell Line Construction. Lentivirus production was carried out in HEK293T cells. Briefly, 293T cells were co-transfected with packaging plasmids (VSV-G, DR8.9) and pCDH lentiviral expression vector or lentiviral shRNA plasmids. At 48 h post transfection, the medium was harvested and filtered. HEK293T, Irf3^{-/-} Irf7^{-/-} mouse embryonic fibroblasts (MEFs), Caco-2, LoVo and A549 cells were infected with lentivirus-containing medium, with polybrene (8 ug/ml) and centrifugation at 1800 rpm for 50 min at 30° C. Cells were incubated at 37° C. for 6 h, and replaced with fresh DMEM with 10% FBS. At 48 h post infection, cells were selected with puromycin (1-2 μg/ml) or hygromycin (200 μg/ml) and stable cell lines were maintained with DMEM containing corresponding antibiotics.

To establish IRF3 knockout cell line, 293T cells were transduced with lentivirus expressing sgRNA targeting IRF3 (pL-CRISPR.EFS.PAC-Targeting-IRF3, Table 3) and selected with 1 μg/ml puromycin. Single colonies were isolated and screened by immunoblotting with IRF3 antibody.

Dual-Luciferase Reporter Assay. HEK293T cells in 24-well plates (˜50% cell density) were transfected with reporter plasmid cocktail containing 50 ng luciferase reporter plasmids (ISRE-luc, IFN-β-luc or NF-κB), 5 ng TK-renilla luciferase reporter (control vector) and the indicated expression plasmids by calcium phosphate precipitation. Whole cell lysates were prepared at 24-30 h post-transfection, and used for dual luciferase assay according to the manufacturer's instruction (Promega).

Confocal Microscopy Analysis. Irf3^{-/-} Irf7^{-/-} MEFs reconstituted with Flag-IRF3 wild-type, Flag-IRF3-N85D were infected with or without SeV (100 HA units/ml). Sixteen hours later, cells were washed, fixed according to methods well known in the art. Cells were incubated with primary mouse monoclonal anti-Flag antibody and Alex Fluor 488-congugated goat secondary antibody, and analyzed with confocal microscope (Leica).

Protein Expression and Purification. HEK293T cells were transfected with plasmids containing Flag-tagged or GST-Tagged genes of interest. Cells were harvested at 48 h post-transfection, and lysed in Triton X-100 buffer (20 mM Tris, pH 7.5, 150 mM NaCl, 1 mM EDTA, 20 mM β-glycerophosphate, 10% glycerol) supplemented with a protease inhibitor cocktail (Roche). Whole cell lysates (WCLs) were sonicated, incubated at 4° C. for 30 min on a rotator, and centrifuged at 12,000 rpm for 30 min. Supernatant was filtered, and precleared with sepharose 4B agarose beads (Thermo) at 4° C. for 1 h. The pre-cleared WCLs were incubated with anti-FLAG M2 agarose beads or glutathione-conjugated agarose beads at 4° C. for 4 h. Anti-FLAG M2 magnetic beads were washed extensively with lysis buffer and eluted with 0.2 mg/ml 3×Flag peptide. GST beads were extensively washed and used immediately for in vitro on-column deamidation assay. Concentration of purified proteins was analyzed by SDS-PAGE and Coomassie staining, with BSA as a standard.

Two-dimensional Gel Electrophoresis. Cells (1×10⁶) were resuspended in 150 μl rehydration buffer (8 M Urea, 2% CHAPS, 0.5% IPG buffer, 0.002% bromophenol blue), sonicated three times, and incubated for 15 min on ice. Whole cell lysates were centrifuged at 12,000 g for 15 min. Supernatants were loaded to IEF strips for isoelectric focusing with a program comprising: 20 V, 10 h (rehydration); 500 V, 1 h; 1000 V, 1 h; 1000-5000 V, 4 h; 5000 V, 4 h. Then, strips were incubated with SDS equilibration buffer (50 mM Tris-HCl [pH8.8], 6 M urea, 30% glycerol, 2% SDS, 0.001% Bromophenol Blue) containing 10 mg/ml DTT for 15 min and SDS equilibration buffer containing 2-iodoacetamide for 15 min. Strips were washed with SDS-PAGE buffer, resolved by SDS-PAGE, and analyzed by immunoblotting.

In vitro Deamidation Assay. Expression plasmids containing IRF3-WT-GST, IRF3-N85D-GST, Flag-CTPS1 were transfected into HEK293T cells. Cell lysates were prepared at 48 h post transfection and proteins were purified with Flag M2 agarose beads (Sigma) or glutathione-conjugated agarose beads (Sigma). In vitro on-column deamidation of IRF3 was performed as previously reported (Zhao et al., 2020, Cell Metab 31, 937-955 e937). Briefly, 0.2 μg of CTPS1 and 0.6 μg of IRF3-WT-GST or IRF3-N85D-GST (on beads) were added to a total volume of 50 μl. The reaction was carried out at 37° C. for 45 min in deamidation buffer (50 mM Tris-HCl at pH 8.0, 20 mM MgCl₂, 5 mM KCl, 1 mM ATP, 1 mM GTP). IRF3-WT-GST or IRF3-N85D-GST were eluted with rehydration buffer (8 M Urea, 2% CHAPS, 0.5% IPG Buffer, 0.002% bromophenol blue) at room temperature and analyzed by two-dimensional gel electrophoresis and immunoblotting.

Mass Spectrometry Analysis for Deamidation Sites. To identify deamidation sites, HEK293T cells were transfected with a plasmid containing IRF3-GST without or with that containing the enzyme-deficient CTPS1 mutant (Flag-CTPS1-ED). Transfected cells were harvested at 48 h post transfected and IRF3-GST was purified with glutathione-conjugated agarose beads from whole cell lysates. Purified proteins were subjected to SDS-PAGE and Coomassie blue staining. Gel slices containing IRF3-GST were prepared for in-gel digestion and mass spectrometry analysis (Poochon Scientific).

Metabolic Profiling and Isotope Tracing. Caco2 cells were mock-infected or infected with SARS-CoV-2 at MOI=1. Cells were harvested at 6 h, 24 h, 48 h and 72 h post-infection for metabolite analysis.

To analyze the effect of SARS-CoV-2 proteins on nucleotide synthesis, LoVo and Caco2 stable cell lines stably expressing SARS-CoV-2 ORF7b, ORF8 and NSP8 were cultured with medium containing [¹⁵N]glutamine for 30 min and 1 h. Cells were washed with 1 ml ice-cold ammonium acetate (NH₄AcO, 150 mM, pH 7.3), added 1 ml −80° C. cold MeOH, and incubated at −80° C. for 20 min. After incubation, cells were scraped off and supernatants were transferred into microfuge tubes. Samples were pelleted at 4° C. for 5 min at 15k rpm. The supernatant was transferred into new microfuge tubes, dried at room temperature under vacuum, and re-suspended in water for LC-MS run.

Samples were randomized and analyzed on a Q-Exactive Plus hybrid quadrupole-Orbitrap mass spectrometer coupled to Vanquish UHPLC system (Thermo Fisher). The mass spectrometer was run in polarity switching mode (+3.00 kV/−2.25 kV) with an m/z window ranging from 65 to 975. Mobile phase A was 5 mM NH₄AcO, pH 9.9, and mobile phase B was acetonitrile. Metabolites were separated on a Luna 3 μm NH2 100 NH2 100A° (150×2.0 mm) column (Phenomenex). The flow rate was 0.3 ml/min, and the gradient was from 15% A to 95% A in 18 min, followed by an isocratic step for 9 min and re-equilibration for 7 min. All samples were run in biological triplicate. Metabolites were detected and quantified as area under the curve based on retention time and accurate mass (5 ppm) using the TraceFinder 4.1 (Thermo Scientific) software. Raw data was corrected for naturally occurring ¹⁵N abundance.

CTPS1 Enzymatic Activity Assay. Flag-CTPS1 expressed 293T stable cell line was transfected with plasmids containing SARS-CoV-2 ORF7b, ORFS, NSP8 and empty vector for 40 h. CTPS1 was purified with anti-FLAG M2 agarose via one-step affinity chromatography. CTPS1 activity was determined by measuring the conversion of UTP to CTP via mass spectrometry. The standard reaction mixture containing 50 mM Tris-HCl (pH 8.0), 10 mM MgCl₂, 10 mM 2-mercaptoethanol, 2 mM L-glutamine, 1 mM GTP, 1 mM ATP, 1 mM UTP, and an appropriate dilution of CTPS1 in a total volume of 50 μl. The reactions were equilibrated to 37° C. for 45 min, and quenched by adding 250 μl cold (−80° C.) methanol and incubating at −80° C. for 20 min. The metabolites were analyzed by LS-MS as described above.

Enzyme-linked Immunosorbent Assay (ELISA). Control and CTPS1-depleted 293T or THP1 cells, Irf3^{-/-} Irf7^{-/-} MEF reconstituted with IRF3-WT, IRF3-N85D, IRF3-N85A and empty vector were infected with SeV (100 HAU/ml). Medium of cells was harvested at the indicated time points. Human IFN-β, CCLS, and mouse IFN-β, CXCL10 were analyzed by commercial ELISA kits according to the manufacturer's instruction.

Electrophoresis Mobility Shift Assay (EMSA). Flag-IRF3-WT and Flag-IRF3-N85D were purified from HEK293T cells. Binding reactions were carried out in 20 μl volumes containing: 2.5 nM P32 labeled DNA probe targeting IFN-β promoter (forward: GCACCGCTAACCGAAACCGAAACTGTGC (SEQ ID NO: 41); reverse: GCACAGTTTCGGTTTCGGTTAGCGGTGC) (SEQ ID NO: 42); 10 mM Tris pH 7.5; 50 mM NaCl; 2 mM DTT and indicated purified Flag-IRF3-WT or Flag-IRF3-N85D. Unlabeled probe (cold probe) was used for competition assay. Reactions were incubated at room temperature for 45 min, and resolved in 6% polyacrylamide gels (29:1 crosslinking) with 0.5×TBE running buffer at 200 V/cm on ice until the loading dye front reached the bottom of the gel. Gels were dried and analyzed using phosphorimaging instrumentation.

Small Molecule Synthesis. Reagents and solvents were obtained from commercial suppliers and used without further purification, unless otherwise stated. Flash column chromatography was carried out using an automated system (Teledyne Isco CombiFlash. Reverse phase high performance liquid chromatography (RP-HPLC) was carried out on a Shimadzu HPLC system. All anhydrous reactions were carried out under nitrogen atmosphere. NMR spectra were obtained on Varian VNMRS-500, VNMRS-600, or Mercury-400.

### (1) Synthesis of Compounds AE-1-10, BE-2-10, and C1-C9:

Step 1: X (1 eq.) and Y (1.2 eq.) were dissolved in DCM/DMF (4:1). HBTU (4 eq.) and triethylamine (4 eq.) were added and the solution was allowed to stir at room temperature for 16 hours. After 16 hours, the reaction mixture was diluted with EtOAc and washed with 10% Na₂CO₃ followed by brine 3 times. The organic layer was dried with Na₂SO₄ and concentrated by rotary evaporation. The residue was purified via flash column chromatography (EtOAc/Hex). All products moved to Step 2 except for the C2 intermediate, which first moved to Step 1.2.

TABLE 2
Synthesis of target compounds.

| Compound | X | Y |
|---|---|---|
| AE-1-10 | 3-nitrobenzoic acid | m-anisidine |
| BE-1-10 | 3-nitrobenzoic acid | 3-(prop-2ynyloxy)aniline |
| C1 | 3-nitrobenzoic acid | Cyclohexamine |
| C2 | 3-nitrobenzoic acid | m-anisidine |
| C4 | 3-nitrobenzoic acid | p-anisidine |
| C5 | 3-nitrobenzoic acid | o-anisidine |
| C6 | 3-nitrobenzoic acid | bis(1h-imidazol-2-amine; |
| | | sulfuric acid |
| C7 | 5-nitrothiophene-2- | Aniline |
| | carboxylic acid | |
| C8 | 4-nitrothiophene-2- | Aniline |
| | carboxylic acid | |
| C9 | 3-nitrobenzoic acid | Amylamine |

Step 1.2 (only applies to C2): The C2 intermediate from Step 1 (1 eq) was dissolved in dry THF and cooled to 0° C. NaH (60% in oil) (1.5 eq) was added portion wise to the stirring solution. Methyl iodide (1.1 eq.) was added drop wise. The reaction mixture was allowed to warm to room temperature. The round bottom was transferred to oil bath and refluxed at 75° C. for 2 hrs. The reaction mixture was poured in ice water and was extracted with EtOAc 3 times. The organic layer was washed with brine 2 times, dried with Na₂SO₄, and concentrated by rotary evaporation. The residue was purified via flash column chromatography and the intermediate moved onto Step 2.

Step 2: Intermediates from Steps 1 and 1.2 (1 eq.) were dissolved in MeOH. Zn (5 eq.) and NH₄Cl (5 eq.) were added, and the mixture was allowed to stir at room temperature for 16 hr. The reaction mixture was dissolved in EtOA and washed with 10% Na₂CO₃ followed by brine 3 times. The organic layer was dried over NaSO₄ and concentrated by rotary evaporation to yield the intermediates which were used in the next reaction without further purification.

Step 3: Intermediates from Step 3 (1 eq.) were dissolved in anhydrous DCM/THF (1:4) under nitrogen gas. DIPEA (1.2 eq) was added via syringe. Chloroacetyl chloride (1.2 eq) was added via syringe slowly dropwise. The reaction mixture was allowed to stir overnight. The mixture was diluted with EtOAc and washed with 10% Na₂CO₃. The organic layer was dried over NaSO₄ and the residue was purified via flash chromatography (EtOAc/Hexane) to yield the final products.

(2) Synthesis of BE-2-10-biotin. BE-2-10 (1 eq.) and PEG-2-biotin-azide (1.1 eq.), were dissolved in DMF. 0.5 M CuSO₄ (0.2 eq), 0.5 M sodium ascorbate (0.2 eq), and 0.5 M TBTA (0.2 eq) were added. The reaction was allowed to stir for 24 hours. The reaction mixture was added to water and extracted 5 times with EtOAc. The residue was recrystallized with ether to yield BE-2-10-biotin.

Drug Treatment. For SARS-CoV-2 infection, Caco-2 or NHBE cells were pre-treated with AE-1-10 or its derivatives (C2, C4, C5, C7, C8 and C9) for 2 h. Then the medium were removed. Cells were washed and infected with SARS-CoV-2. Afterwards, medium containing virus was removed, and cells were cultured with drug-containing medium. Drugs were added at each 24 h after viral infection until the end of the experiments. DMSO was used as control. To test the effect of AE-1-10 on IRF3 deamidation, ORF8 or Nsp8 expressed Caco-2 or A549 cells were treated with 5 μM AE-1-10 for 4 h. To analyze the effect of AE-1-10 on intracellular metabolites, ORF8 expressed Caco-2 or LoVo cells were treated with 5 μM AE-1-10 for 2 h.

TABLE 3
Primer Pairs

| | | |
|---|---|---|
| Gene Target | Forward | Reverse |

Real-time PCR primers for human genes

| | | |
|---|---|---|
| IFNB1 | CTTTCGAAGCCTTTGCTCTG (SEQ ID | CAGGAGAGCAATTTGGAGGA |
| | NO: 1) | (SEQ ID NO: 2) |
| Mx1 | GGTGGTGGTCCCCAGTAATG (SEQ ID | ACCACGTCCACAACCTTGTCT |
| | NO: 3) | (SEQ ID NO: 4) |
| ISG15 | GTGGACAAATGCGACGAACCCC (SEQ | TCGAAGGTCAGCCAGAACAG |
| | ID NO: 5) | (SEQ ID NO: 6) |
| ISG56 | TCTCAGAGGAGCCTGGCTAA (SEQ ID | TGACATCTCAATTGCTCCAG |
| | NO: 7) | (SEQ ID NO: 8) |
| CCL5 | CCTGCTGCTTTGCCTACATTGC (SEQ | ACACACTTGGCGGTTCTTTCGG |
| | ID NO: 9) | (SEQ ID NO: 10) |
| CTPS1 | AGCTTGGCAGAAGCTCTGTA (SEQ ID | CCAACTGCATCCCTAAGCAC |
| | NO: 11) | (SEQ ID NO: 12) |
| β-actin | GTTGTCGACGACGAGCG (SEQ ID NO: | GCACAGAGCCTCGCCTT (SEQ ID |
| | 13) | NO: 14) |

ChIP-qPCR primers for mouse Ifn genes

| | | |
|---|---|---|
| Ifnbl promoter | CCAGGAGCTTGAATAAAATGAA (SEQ | TGCAGTGAGAATGATCTTCCTT |
| | ID NO: 15) | (SEQ ID NO: 16) |
| Ifna4 promoter | ATCCCAGACACACAAGCAGAGAG | GGCTGTGGGTTTGAGTCTTCT |
| | (SEQ ID NO: 17) | (SEQ ID NO: 18) |

q-PCR primers for mouse genes

| | | |
|---|---|---|
| Ifnb1 | CCCTATGGAGATGACGGAGA (SEQ ID | CCCAGTGCTGGAGAAATTGT |
| | NO: 19) | (SEQ ID NO: 20) |
| Ifna4 | GCAGAAGTCTGGAGAGCCCTC (SEQ | TGAGATGCAGTGTTCTGGTCC |
| | ID NO: 21) | (SEQ ID NO: 22) |
| Isg15 | TCCATGACGGTGTCAGAACT (SEQ ID | GACCCAGACTGGAAAGGGTA |
| | NO: 23) | (SEQ ID NO: 24) |
| Cxcl10 | CCTGCCCACGTGTTGAGAT (SEQ ID | TGATGGTCTTAGATTCCGGATTC |
| | NO: 25) | (SEQ ID NO: 26) |
| Mx1 | GTGGTAGTCCCCAGCAATGT (SEQ ID | TGCTGACCTCTGCACTTGAC |
| | NO: 27) | (SEQ ID NO: 28) |
| Isg56 | CAAGGCAGGTTTCTGAGGAG (SEQ ID | GACCTGGTCACCATCAGCAT |
| | NO: 29) | (SEQ ID NO: 30) |
| Gapdh | GTTGTCTCCTGCGACTTC (SEQ ID NO: | GGTGGTCCAGGGTTTCTTA (SEQ |
| | 31) | ID NO: 32) |

Real-time PCR primers for SARS-CoV-2 genes

| | | |
|---|---|---|
| Nsp1 | ACACGTCCAACTCAGTTTGC (SEQ ID | CGAGCATCCGAACGTTTGAT |
| | NO: 33) | (SEQ ID NO: 34) |
| E | ACTTCTTTTTCTTGCTTTCGTGGT (SEQ | GCAGCAGTACGCACACAATC |
| | ID NO: 35) | (SEQ ID NO: 36) |
| N | GGGGAACTTCTCCTGCTAGAAT (SEQ | GGGGAACTTCTCCTGCTAGAAT |
| | ID NO: 37) | (SEQ ID NO: 38) |

sgRNA primers for Human IRF3

| | |
|---|---|
| IRF3 gRNA1 | CTGGTGCATATGTTCCCGGGAGG (SEQ ID NO: 39) |
| IRF3 gRNA2 | GCCGTAGGCCGTGCTTCCAAGGG (SEQ ID NO: 40) |

### Example 2

### Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/mL) | mg/mL |
| ‘Compound X’ (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1 N Sodium hydroxide solution | q.s. |
| (pH adjustment to 7.0-7.5) | |
| Water for injection | q.s. ad 1 mL |
| (vi) Aerosol | mg/can |
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |
| (vii) Topical Gel 1 | wt. % |
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | wt. % |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | wt. % |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | wt. % |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | wt. % |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 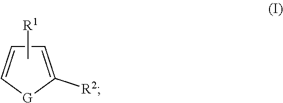
wherein,
G is 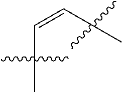 [SMILES: */C=C\*]
or S;
R¹ is —NH(C═O)CH₂X wherein X is a leaving group;
R² is —(C═O)NR^{a}R³ or —NR^{a}(C═O)R³, wherein R^{a} is H or —(C₁-C₆)alkyl;
R³ is —(C₁-C₆)alkyl, —(C₃-C₆)cycloalkyl, phenyl-R⁴, or 5- or 6-membered heteroaryl; and
R⁴ is —O(C₁-C₆)alkyl, —(C₁-C₆)alkyl, halo, or H;
wherein R¹ is in the beta-position relative to R², and each (C₁-C₆)alkyl moiety is independently saturated or unsaturated and optionally interrupted by a heteroatom.

2. The compound of claim 1 wherein the compound is represented by Formula II:

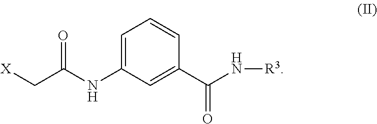

3. The compound of claim 1 wherein the compound is represented by Formula III:

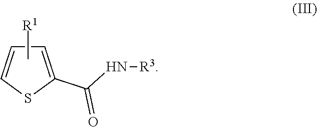

4. The compound of claim 3 wherein le is at the 4-position.

5. The compound of claim 3 wherein le is at the 5-position.

6. The compound of claim 1 wherein X is halo, N₃, methanesulfonate (OMs), or p-toluenesulfonate (OTs).

7. The compound of claim 1 wherein X is chloro.

8. The compound of claim 1 wherein R³ is pentyl, phenyl-OCH₃, phenyl-H, or imidazolyl.

9. The compound of claim 1 wherein R³ is phenyl-R⁴ and R⁴ is ortho-OCH₃.

10. The compound of claim 1 wherein the compound is: 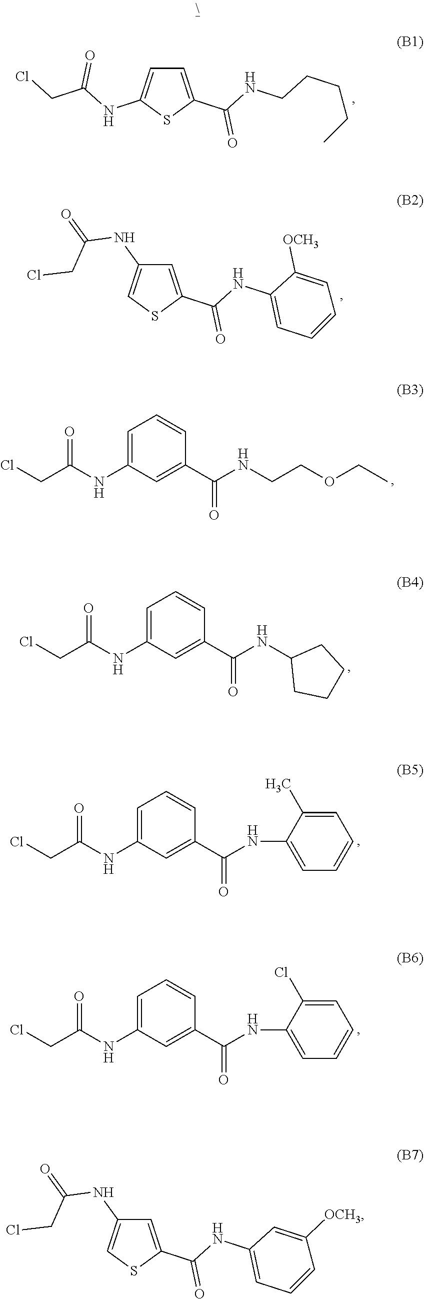 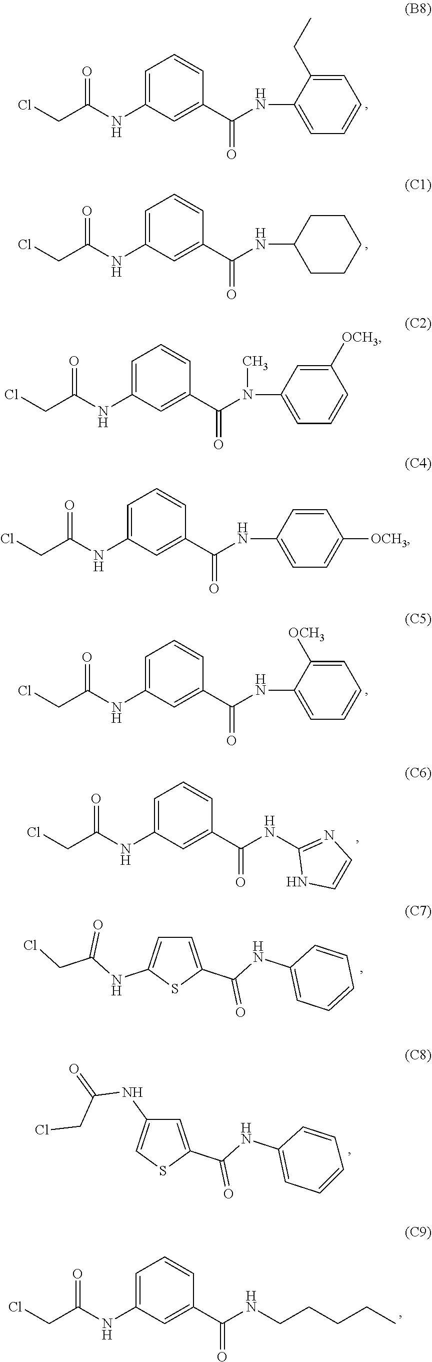 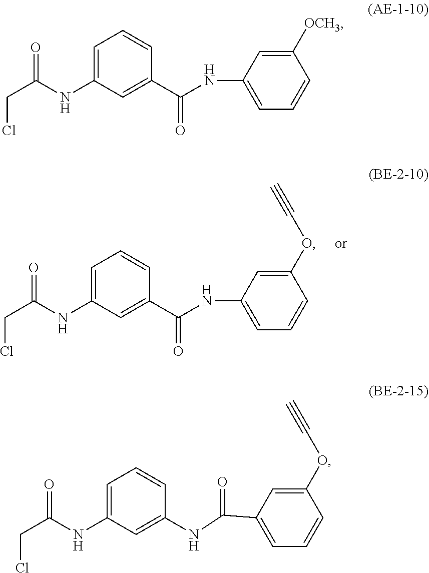 [SMILES: COc1cccc(NC(=O)c2cccc(NC(=O)CCl)c2)c1 ; C#COc1cccc(NC(=O)c2cccc(NC(=O)CCl)c2)c1 ; C#COc1cccc(C(=O)Nc2cccc(NC(=O)CCl)c2)c1]
or a pharmaceutically acceptable salt thereof.

11. The compound of claim 10 wherein the compound is:

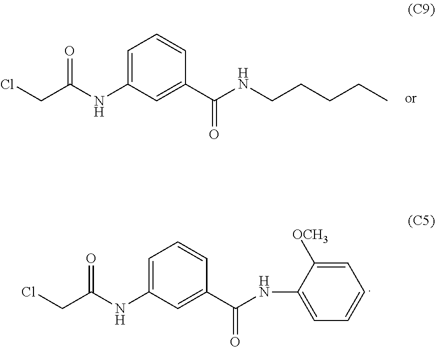 [SMILES: CCCCCNC(=O)c1cccc(NC(=O)CCl)c1 ; COc1ccccc1NC(=O)c1cccc(NC(=O)CCl)c1]

12. The compound of claim 1 wherein the compound is:

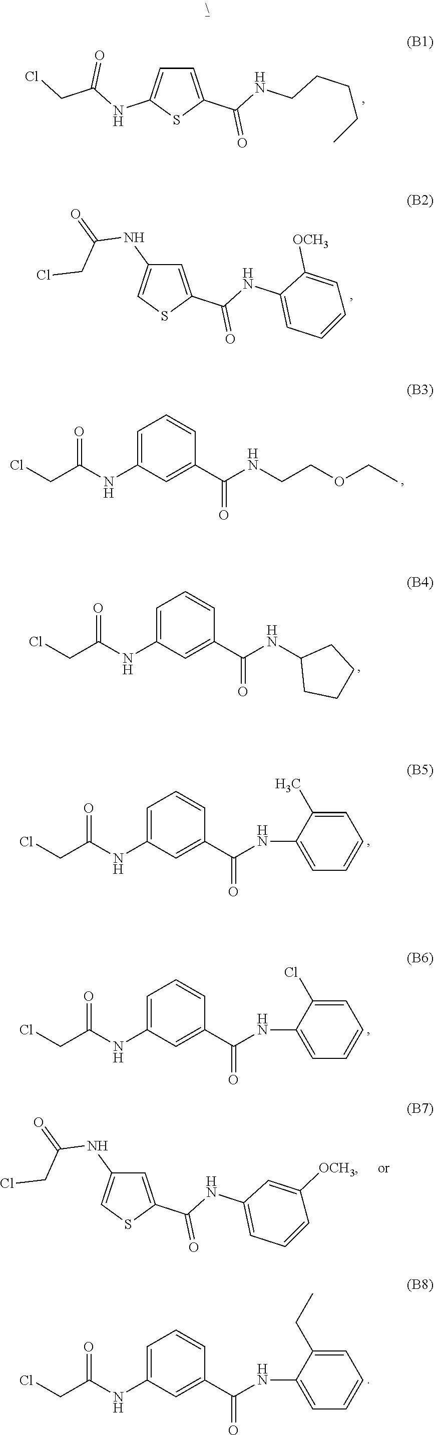

13. A pharmaceutical composition comprising a compound of claim 1 and a pharmaceutically acceptable excipient.

14. A method for antiviral treatment comprising administering to a subject in need thereof a therapeutically effective amount of the composition of claim 13, thereby inhibiting replication of a virus that has infected the subject.

15. The method of claim 14 wherein the method comprises administering the composition in combination with one or more additional therapeutic agents; wherein the combination is administered simultaneously or sequentially.

16. The method of claim 14 wherein the virus is a coronavirus.

17. The method of claim 16 wherein the coronavirus is Severe Acute Respiratory Syndrome Coronavirus 2 (SARS-CoV-2), Middle East Respiratory Syndrome Coronavirus (MERS-CoV), or Severe Acute Respiratory Syndrome Coronavirus (SARS-CoV).

18. The method of claim 14 wherein the compound is an inhibitor of cytidine triphosphate synthetase 1 (CTPS1).

19. A method of treating a human subject identified as having or suspected of having COVID-19, the method comprising administering to the subject an effective amount of a cytidine triphosphate synthetase 1 (CTPS1) inhibitor effective to reduce interferon regulatory factor 3 (IRF3) deamidation, thereby treating the subject.

20. The method of claim 19 wherein the CTPS1 inhibitor is a compound of claim 1.
